(19) United States
(12) Patent Application Publication
(10) Pub. No.: US 2011/0206727 A1
(43) Pub. Date: Aug. 25, 2011

(54) Multivalent Vaccines Based on Papaya Mosaic Virus and Uses Thereof

(76) Inventor: Denis Leclerc, Fossambault-sur-le Lac (CA)

(21) Appl. No.: 13/056,962

(22) PCT Filed: May 15, 2009
(86) PCT No.: PCT/CA09/00636

§ 371 Date: May 2, 2011

Related U.S. Application Data
(60) Provisional application No. 61/084,803, filed on Jul. 30, 2008.

(51) Int. Cl. A61K 39/295; A61P 37/04; A61P 31/16

(52) U.S. Cl. 424/201.1; 424/202.1

(57) ABSTRACT

A multivalent vaccine composition that comprises a papaya mosaic virus (PapMV) component and one or more antigens is provided. The composition can further optionally comprise a Salmonella spp. porin component. The PapMV component can be PapMV or PapMV virus-like particles (VLPs). The porin component can be a Salmonella spp. OmpC, OmpF or a combination thereof, and can be combined with the PapMV component or conjugated to the PapMV component. The PapMV component in the multivalent vaccine composition functions as an adjuvant and/or an immunostimulant with respect to the one or more antigens. Use of the multivalent vaccine compositions to provide protection against a plurality of strains of a pathogen, or against more than one pathogen, are also provided.

### FIELD OF THE INVENTION

The present invention relates to the field of vaccine formulations and, in particular, to multivalent vaccines containing plant virus particles.

### BACKGROUND OF THE INVENTION

Multivalent vaccines (also known as polyvalent vaccines) are vaccines that provide provide protection against more than one strain of pathogen, or that provide protection against more than one pathogen. In the latter context, multivalent vaccines provide the advantage of decreasing the number of separate vaccinations required by an individual. This can be particularly useful, for example, when vaccinating babies or children.

Challenges in developing multivalent vaccines include ensuring that enhancement of the immune response to one of the component antigens does not compromise the immune response to the other component antigen(s) to the extent that they are no longer effective.

Influenza virus infections cause 36,000 deaths and 114,000 hospitalizations per year in the USA alone. Most modern influenza vaccines are targeted to type A or B viruses, and predominantly to type A. The segmented nature of the influenza virus genome allows entire genes to be exchanged between different strains of the virus, which leads to the emergence of new virulent strains and makes prophylaxis and/or treatment challenging. The multiplicity of influenza virus strains confers the need for annual vaccination according to the strains that are more prevalent at the time, which is determined each year by the World Health Organization.

Existing human vaccines against the influenza virus contain three killed or attenuated virus strains—one A (H3N2) virus, one A (H1N1) virus, and one B virus. The hemagglutinin (HA) and the neuraminidase (NA) proteins, which are accessible large glycoproteins at the surface of the virus, are the major target of the immune response during infection which has in turn induced a drift and shift in these proteins (Fier et al., 2004, Virus Research 103:173-176). The selection pressure of the immune system on the surface glycoproteins favours the emergence of mutated viruses that propagate efficiently and cause new epidemics. The newly emerged strain is usually selected as a component of the next vaccine, but this may not come onto the market until 6-8 months later. During this time, the circulating virus has time to evolve which results in a partial efficiency of the vaccine. The reassortment of the viruses in pig and bird reservoirs complicates the cycle and can be the source of pandemics.

A live attenuated nasal vaccine against influenza (FluMist® from MedImmune Vaccines, Inc.) may provide a certain level of cross protection to other strains of influenza through induction of a cytotoxic T lymphocyte (CTL) response toward highly conserved protein found inside the virus particle (Kaiser, 2006, Science 312:380-383). One approach to a universal flu vaccine, therefore, is to use conserved internal proteins such as the matrix protein (M1) or the nucleocapsid (NP) to elicit immunity based on CTL rather than neutralizing antibodies to HA and NA. The injection of purified NP or M1 is unlikely to mount alone a protective CTL response, but rather the target proteins must be associated with an adjuvant or a delivery system that is aimed at developing a CTL response to a conserved epitope, such as adenoviral vectors (Bangari and Mittal, 2006, Curr Gene Ther 6:215-226; Ghosh et al., 2006, Appl Biochem Biotechnol 133:9-29) and DNA vaccines (Laddy and Weiner, 2006, Int Rev Immunol 25:99-123; Stan et al., 2006, Hematol Oncl Clin North Am 20:613-636). Adenoviral vectors, however, can be neutralized by antibodies that inhibit their entry to APC (Palker et al., 2004, Virus Res 105:183-194) and DNA vaccines developed to date are not immunogenic in large animals and require addition of an adjuvant, such as CpG, to increase their immunogenicity (Klinman D. M., (2006) Int Rev Immunol. 25; 135-154).

Among the numerous new approaches to vaccine development, virus-like-particles (VLPs) made of viral nucleocapsids have emerged as a potential strategy. Influenza virus VLPs have been described (see International Patent Application No. PCT/US2004/022001 (WO 2005/020889); and U.S. Patent Application Nos. 2005/0186621 and 2005/0009008). Chemical cross-linking of the influenza M2e peptide to Hepatitis B Virus (HBV) (Jegerlehner et al., 2002, Vaccine 20:3104-3112) or to Human Papillomavirus (HPV) (Ionescu et al., J. Pharm. Sci. 95:70-79) and an in-frame fusion of M2e peptide with HBV coat protein (Neirynck et al., 1999, Nat Med 5:1157-1163; Jegerlehner et al., 2004, Vaccine 22:3104-3112; and U.S. Patent Application No. 2003/0202982) were all successful at protecting mice against challenge with influenza virus. A chimeric VLP made from the yeast retrotransposon Ty p1 protein fused to an influenza virus haemagglutinin or nucleoprotein epitope has also been described (U.S. Pat. No. 6,060,064).

International Patent Application No. PCT/CA2007/002069 (WO 2008/058396) describes an antigen-presenting system (APS) comprising one or more influenza virus antigens in combination with a papaya mosaic virus (PapMV) or a virus like particle (VLP) derived from papaya mosaic virus that can be used as a vaccine against influenza.

Typhoid fever, a serious systemic infection, is caused by an acute infection of the reticuloendothelial system with the enterobacterium Salmonella enterica serovar Typhi (S. typhi)). Vaccines against typhoid fever have been developed. The oral live attenuated galE mutant Ty21a (Vivotif® vaccine; Berna Biotech, Ltd., Berne, Switzerland) is effective in endemic areas, but it is not licensed for use in children younger than six years old. Also, three to four doses are required to reach a partial protective immune response (Levine et al., 1999, Vaccine 1: S22-S27). Vi capsular polysaccharide vaccine (ViCPS) (Typhim Vi™; Aventis Pasteur) is licensed for children over 2 years old; one injection of Vi provides similar protection to the Ty21a vaccine, but only for a short period. The lack of long lasting immunity is the major disadvantage of this vaccine (Lin et al., 2001, N Engl J. Med. 344:1263-9; Sabitha et al., 2004, Indian J. Med. Sci. 58:141-149). Furthermore, the best protection achieved for these vaccines typically ranges between 50% and 80% of recipients (Crump et al., 2004, Bulletin of the World Health Organisation, 82; 346-353; Levine et al., 1999, Vaccine 1: S22-S27; Lin et al., 2001, N Engl J. Med. 344:1263-9; Sabitha et al., 2004, Indian J. Med. Sci. 58:141-149). A new candidate vaccine based on an attenuated strain of S. typhi (Ty-800) has been developed by Emergent BioSolutions, Inc. (Gaithersburg, Md.). This vaccine candidate is currently in phase II clinical trials in Vietnam, an endemic area (Crump et al., 2004, ibid.). As with the Ty21a vaccine, this vaccine is administered by the oral route and will likely not be suitable for administration to children.

More recently, researchers have found that porins are important antigens for the induction of specific protective immune responses against infection caused by several gram-negative bacteria (Humphries et al., 2006, Vaccine 24:36-44; Kim et al., 1999, J. Immunol. 162:6855-6866). Porins are trimeric exposed outer membrane proteins (OMPs) of gram-negative bacteria that function as relatively nonspecific channels, allowing small hydrophilic molecules to pass across the outer membrane (Nikaido, 2003, Microbiol Mol Biol Rev. 67:593-656). Immune responses to porins appear to involve both the humoral and cell-mediated immune pathways. Typhoid fever acute and convalescent patients, for example, show high levels of porin-specific antibodies (Calderon et al., 1986, Infect. Immun. 52:209-212, Ortiz, et al., 1989, J. Clin. Microbiol. 27:1640-1645). In addition, typhoid fever patients and human volunteers immunized with Ty21a oral vaccine have shown porin-specific cellular immune responses (Salerno-Goncalves, et al., 2002, J. Immunol. 169:2196-203). OmpC and OmpF, two key S. typhi porins, have been shown to raise a long-lasting antibody response in mice (Secundino et al., 2006, Immunology 117:59).

Virus-like particles (VLPs) derived from the coat protein of papaya mosaic virus (PapMV) and their use as immunopotentiators has been described (International Patent Application No. PCT/CA03/00985 (WO 2004/004761)). Expression of the PapMV coat protein in E. coli leads to the self-assembly of VLPs composed of several hundred CP subunits organised in a repetitive and crystalline manner (Tremblay et al., 2006, FEBS J 273:14). Studies of the expression and purification of PapMV CP deletion constructs further indicate that self-assembly (or multimerization) of the CP subunits is important for function (Lecours et al., 2006, Protein Expression and Purification, 47:273-280; Denis et al., 2007, Virology 363; 59-68). The ability of PapMV VLPs comprising epitopes from either gp100 or the influenza virus M1 protein have been shown to induce MHC class I cross-presentation of the epitopes leading to expansion of specific human T cells (Leclerc, D., et al., J. Virol, 2007, 81(3):1319-26; Epub. ahead of print Nov. 22, 2006).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide multivalent vaccines based on papaya mosaic virus and uses thereof. In accordance with one aspect of the invention, there is provided a multivalent vaccine composition comprising a papaya mosaic virus (PapMV) component, one or more antigens, and optionally a porin component, said PapMV component comprising PapMV or PapMV virus-like particles (VLPs) derived from PapMV coat protein, and said porin component comprising a Salmonella spp. OmpC, OmpF or a combination thereof.

In accordance with another aspect of the invention, there is provided a use of a multivalent vaccine composition comprising a PapMV component, one or more antigens, and optionally a porin component, said PapMV component comprising PapMV or PapMV VLPs derived from PapMV coat protein, and said porin component comprising a Salmonella spp. OmpC, OmpF or a combination thereof, for inducing an immune response against a plurality of pathogens in an animal.

In accordance with another aspect of the invention, there is provided a use of a multivalent vaccine composition comprising a PapMV component, one or more antigens, and optionally a porin component, said PapMV component comprising PapMV or PapMV VLPs derived from PapMV coat protein, and said porin component comprising a Salmonella spp. OmpC, OmpF or a combination thereof, wherein said multivalent vaccine composition comprises PapMV VLPs and said one or more antigens are one or more influenza virus antigens, for inducing an immune response against influenza virus in an animal.

In accordance with another aspect of the invention, there is provided a use of an effective amount of a PapMV component, one or more antigens and optionally a porin component in the manufacture of a multivalent vaccine composition, wherein said PapMV component comprises PapMV or PapMV VLPs derived from PapMV coat protein, and said porin component comprises a Salmonella spp. OmpC, OmpF or a combination thereof.

In accordance with another aspect of the invention, there is provided a method of inducing an immune response against one or more pathogens in an animal, said method comprising administering to said animal an effective amount of a multivalent vaccine composition comprising a papaya mosaic virus (PapMV) component, one or more antigens, and optionally a porin component, said PapMV component comprising PapMV or PapMV virus-like particles (VLPs) derived from PapMV coat protein, and said porin component comprising a Salmonella spp. OmpC, OmpF or a combination thereof.

In accordance with another aspect of the invention, there is provided a use of a composition comprising a papaya mosaic virus (PapMV) component and a porin component as an adjuvant, wherein said PapMV component comprises PapMV or PapMV virus-like particles (VLPs) derived from PapMV coat protein and said porin component comprises a Salmonella spp. OmpC, OmpF or a combination thereof.

In accordance with another aspect of the invention, there is provided a use of a composition comprising PapMV VLPs and optionally a porin component to improve the efficacy of an influenza vaccine whereby a subject treated with said composition and said influenza vaccine shows an improved immune response over a subject treated with said influenza vaccine alone, wherein said porin component comprises a Salmonella spp. OmpC, OmpF or a combination thereof.

In accordance with another aspect of the invention, there is provided a method of improving the efficacy of an influenza vaccine comprising administering to a subject said influenza vaccine and a composition comprising PapMV VLPs and optionally a porin component, whereby the subject treated with said influenza vaccine and said composition shows an improved immune response over a subject treated with said influenza vaccine alone, wherein said porin component comprises a Salmonella spp. OmpC, OmpF or a combination thereof.

### BRIEF DESCRIPTION OF THE DRAWINGS

These and other features of the invention will become more apparent in the following detailed description in which reference is made to the appended drawings.

FIG. 1 presents (A) the amino acid sequence for the papaya mosaic virus coat (or capsid) protein (GenBank Accession No. NP_{—}044334.1; SEQ ID NO:1), (B) the nucleotide sequence encoding the papaya mosaic virus coat protein (GenBank Accession No. NC_{—}001748 (nucleotides 5889-6536); SEQ ID NO:2), and (C) the amino acid sequence of the modified PapMV coat protein CPΔN5 (SEQ ID NO:3).

FIG. 2 presents (A) the amino acid sequence (SEQ ID NO:4) of the OmpC precursor from Salmonella enterica subsp. enterica serovar Typhi Ty2 (GenBank Accession No. P0A264); and (B) the amino acid sequence (SEQ ID NO:5) of the OmpF precursor protein from Salmonella enterica subsp. enterica serovar Typhi CT18 (GenBank Accession No. CAD05399).

FIG. 3 presents (A) the amino acid sequence of the modified PapMV coat protein PapMV CPsm [SEQ ID NO:37]; (B) the nucleotide sequence encoding PapMV CPsm [SEQ ID NO:38]; (C) the amino acid sequence of PapMV coat protein comprising an affinity peptide for binding to OmpC [SEQ ID NO:6], and (D) the amino acid sequence of PapMV coat protein comprising an affinity peptide for binding to OmpF [SEQ ID NO:7]. Differences between the cloned and wild-type sequence are marked in bold and underlined; the affinity peptide sequence is underlined, and the histidine tag is shown in italics.

FIG. 4 presents the results from a challenge experiment in which mice were vaccinated with either OmpC alone or a candidate vaccine comprising PapMV VLPs and OmpC (mixed in a 1:1 w/w ratio) and subsequently challenged with 500LD₅₀ of Salmonella typhi (control mice were vaccinated with PBS and subsequently challenged with 20LD₅₀ of S. typhi) and demonstrates that the candidate vaccine provides excellent protection against the S. typhi challenge.

FIG. 5 presents (A) the total IgG titres against the total Fluviral® proteins, (B) the IgG2a titres against the Fluviral® proteins, and (C) the IgG1 titres against the Fluviral® proteins, measured by ELISA in mice immunised with Fluviral® alone or with Fluviral® in combination with either 3 μg or 30 μg PapMV VLP-OmpC (“PAL-typhoid”).

FIG. 6 presents (A) the total IgG titres, (B) IgG2a titres, and (C) IgG1 titres, against the influenza virus NP protein measured by ELISA in mice immunised with Fluviral® alone or with Fluviral® in combination with either 3 μg or 30 μg PapMV VLP-OmpC (“PAL-typhoid”).

FIG. 7 presents (A) body weight loss and (B) severity of symptoms experienced by mice submitted to a 4LD₅₀ challenge with the WSN/33 strain of influenza after vaccination with Fluviral® alone or with Fluviral® in combination with either 3 μL or 30 μL PapMV VLP-OmpC; (C) presents the survival rate of the mice.

FIG. 8 presents IgG2a titres against the OmpC protein measured by ELISA in mice immunised with Fluviral® alone or with Fluviral® in combination with either 3 μg or 30 μg PapMV VLP-OmpC (“PAL-typhoid”).

FIG. 9 depicts the measure of the antibody response to the Fluviral® proteins in mice innoculated with Fluviral® alone, or Fluviral® adjuvanted with either 3 μg or 30 μg of PapMV VLPs; total IgG (A), IgG2a (B) and IgG1 (C) were measured using serum harvested 14 days after one s.c. immunization.

FIG. 10 depicts the measure of the antibody response to the influenza NP protein in mice innoculated with Fluviral® alone, or Fluviral® adjuvanted with either 3 μg or 30 μg PapMV VLPs; total IgG (A), IgG2a (B) and IgG1 (C) were measured using serum harvested 14 days after one s.c. immunization.

FIG. 11 presents the survival rate of mice submitted to a 4LD₅₀ challenge with the WSN/33 strain of influenza after vaccination with Fluviral® alone or with Fluviral® in combination with either 3 μg or 30 μg PapMV VLPs.

FIG. 12 depicts the measure of the antibody response to the Fluviral® proteins in mice innoculated with Fluviral® alone, or Fluviral® adjuvanted with alum; total IgG (A), IgG1 (B) and IgG2a (C) were measured by ELISA.

FIG. 13 depicts the measure of the antibody response to the influenze NP protein in mice innoculated with Fluviral® alone, or Fluviral® adjuvanted with alum; total IgG (A), IgG1 (B) and IgG2a (C) were measured by ELISA.

FIG. 14 presents (A) the amino acid sequences of the C-terminus of the wild-type PapMV coat protein and recombinant constructs comprising a fusion at the C-terminus of the PapMV coat protein of the affinity peptide to OmpC or to OmpF (constructs PapMV OmpC and PapMV OmpF, respectively); (B) SDS-PAGE showing the profile of the purified proteins PapMV, PapMV OmpC, PapMV OmpF, OmpC and OmpF, [First lane: molecular weight markers, second lane; PapMV VLPs, third lane; PapMV OmpC VLPs, fourth lane; PapMV OmpF VLPs, fifth lane; purified OmpC, sixth lane; purified OmpF]; (C) an electron micrograph of the high-speed pellet of the recombinant PapMV OmpC and PapMV OmpF VLPs; and the high avidity binding of the PapMV VLPs to their respective antigen; (D) presents an ELISA showing the binding the high avidity PapMV OmpC VLPs to the OmpC antigen, and (E) presents an ELISA showing the binding the high avidity PapMV OmpF VLPs to the OmpF antigen.

FIG. 15 presents the results of a protection assay against S. typhi challenge in mice, (A) depicts the protective capacity against 100 LD₅₀ of S. typhi in mice immunized with OmpC alone and mice immunized with a preparation containing OmpC+PapMV OmpC VLPs; (B) depicts the protective capacity against 100 LD₅₀ of S. typhi in mice immunized with OmpF alone and mice immunized with a preparation containing OmpF+PapMV OmpF VLPs; (C) depicts the protective capacity against 500 LD₅₀ of S. typhi in mice immunized with OmpC alone and mice immunized with a preparation containing OmpC+PapMV OmpC VLPs, and (D) depicts the protective capacity against 500 LD₅₀ of S. typhi in mice immunized with OmpF alone and mice immunized with a preparation containing OmpF+PapMV OmpF VLPs.

FIG. 16 illustrates the evaluation of IgG1 (A), IgG2a (B), IgG2b (C) and IgG3 (D) produced in mice in response to immunization with OmpC or a vaccine comprising OmpC+PapMV OmpC VLPs, and (E) illustrates that co-immunization of mice with OmpC and PapMV OmpC followed by challenge with S. typhi favours the long lasting protection against S. typhi infection (as illustrated by % survival) when compared to immunization with OmpC or PapMV OmpC alone.

FIG. 17 illustrates that administration of PapMV increased the protective capacity of OmpC porin in mice; (A) Groups of 10 female BALB/c mice were immunized i.p. with 10 μg of OmpC either alone or in combination with 30 μg of PapMV and a booster was given on day 15 with OmpC alone. Control mice were injected with saline (ISS) or PapMV. The groups were challenged at day 21 with 100 (filled symbols) or 500 (open symbols) LD₅₀ of S. typhi and the survival rate was recorded for 10 days after the challenge. Control groups were challenged with 20 LD₅₀ of S. typhi. A representative result of three experiments is shown. (B) Groups of five female BALB/c mice were immunized i.p. on day 0 with 10 μg of OmpC alone or in combination with either 30 μg of PapMV or Freund's incomplete adjuvant (IFA) (1:1 v/v). On day 15, all mice were boosted with 10 μg of OmpC only. Control mice were injected with isotonic saline solution (ISS). Antibody titres were measured by enzyme-linked immunosorbent assay (ELISA) on day 21 after the first immunization.

FIG. 18 presents the results of a protection assay against S. typhi challenge in mice, (A) depicts the protective capacity of OmpF alone and a preparation containing either OmpF+PapMV OmpF VLPs or OmpF+PapMV against a challenge of 77 LD₅₀ of S. typhi in mice, and (B) depicts the protective capacity of OmpF alone and a preparation containing either OmpF+PapMV OmpF VLPs or OmpF+PapMV against a challenge of 378 LD₅₀ of S. typhi in mice.

FIG. 19 presents (A) the amino acid sequences of the C-terminus of the PapMV SM coat protein and the recombinant construct comprising a fusion of the affinity peptide to OmpC at the C-terminus of the PapMV SM coat protein; (B) SDS-PAGE showing the profile of the purified protein PapMV SM OmpC [First lane: molecular weight markers, second lane; bacterial lysate before induction with IPTG, third lane; bacterial lysation of bacteria expressing high amount of the PapMV SM CP, fourth lane; Purified PapMV SM OmpC protein. PapMV OmpF], and (C) an electron micrograph of the high-speed pellet of the recombinant PapMV SM OmpC at 100,000× magnification.

FIG. 20 presents (A) the amino acid sequence of the PapMV SM coat protein comprising an affinity peptide for binding to OmpC (PapMV SM OmpC) [SEQ ID NO:8], and (B) the nucleotide sequence enconding the PapMV SM OmpC protein [SEQ ID NO:9].

FIG. 21 presents the results of a protection assay against S. typhi challenge in mice, (A) depicts the protective capacity of OmpC alone or a preparation containing either OmpC+PapMV SM OmpC VLPs or OmpC+PapMV VLPs against 105 LD₅₀ of S. typhi in mice, and (B) depicts the protective capacity of OmpC alone or a preparation containing either OmpC+PapMV SM OmpC VLPs or OmpC+PapMV VLPs against 520 LD₅₀ of S. typhi in mice.

FIG. 22 shows (A) total IgG titers, (B) IgG2a titers and (C) IgG1 titers, against the haemagglutinin proteins of Fluviral® vaccine as measured by ELISA for BALB/c mice that received one subcutaneous injection of Fluviral® vaccine (3 μg; equivalent to one-fifth the human dose) or of Fluviral® adjuvanted with either 3 μg or 30 μg of purified OmpC. The red line represents the normal baseline for pre-immunised mice. ***p<0.001 vs. Fluviral®.

FIG. 23 shows (A) total IgG titers, (B) IgG1 titers and (C) IgG2a titers, against the influenza virus NP protein as measured by ELISA for the BALB/c mice treated as described for FIG. 22. The red line represents the normal baseline for pre-immunised mice. *p<0.05 vs. Fluviral® and **p<0.01 vs. Fluviral®.

FIG. 24 presents (A) the change in body weight of BALB/c mice vaccinated with one subcutaneous injection of Fluviral® vaccine (3 μg; equivalent to one-fifth the human dose) or of Fluviral® adjuvanted with 30 μg of purified OmpC, and challenged with 4,000 pfu of influenza strain WSN/33 (weight was measured daily for 14 days after challenge); (B) symptoms presented by the mice scored according to Table 5 on a daily basis for 14 days after challenge, and (C) shows the survival rate of the mice. Only mice immunised with Fluviral® adjuvanted with 30 μg of OmpC survived the challenge.

FIG. 25 shows (A) total IgG titers, (B) IgG2a titers and (C) IgG1 titers, against the haemagglutinin proteins of the Fluviral® vaccine as measured by ELISA for BALB/c mice that received one subcutaneous injection of Fluviral® vaccine (3 μg; equivalent to one-fifth the human dose) or of Fluviral® adjuvanted with either 3 μg or 30 μg of PapMV CPfl3y VLPs.

FIG. 26 shows (A) total IgG titers, (B) IgG2a titers and (C) IgG1 titers, against the influenza virus NP protein as measured by ELISA for BALB/c mice that received one subcutaneous injection of Fluviral® vaccine (3 μg; equivalent to one-fifth the human dose) or of Fluviral® adjuvanted with 3 μg or 30 μg of PapMV CPfl3y VLPs.

FIG. 27 shows (A) the survival rate of BALB/c mice vaccinated with one subcutaneous injection of Fluviral® vaccine (3 μg; equivalent to one-fifth the human dose) or of Fluviral® adjuvanted with 3 μg or 30 μg of PapMV CPfl3y VLPs, and challenged with 4,000 pfu of influenza strain WSN/33; (B) change in body weight of the mice as measured daily for 14 days after challenge, and (C) symptoms presented by the mice on a daily basis for 14 days after challenge.

FIG. 28 shows (A) total IgG; (B) IgG2a and (C) IgG1 to Fluviral® 2008 (log scale); (D) IgG2a against the purified influenza NP protein (log scale); as measured by ELISA for BALB/c mice that received one subcutaneous injection of Fluviral® vaccine (3 μg; equivalent to one-fifth the human dose) or of Fluviral® adjuvanted with 3 μg or 30 μg of rVLP-SM; (E) survival rate of the mice when challenged with 4LD₅₀ of strain WSN/33. Significant differences between the formulation adjuvanted with 30 μg of rVLP-SM and the Fluviral® 2008 treatment alone are shown by the symbol (*). Significant differences between the formulations containing 3 or 30 μg): rVLP-SM are shown by the symbol (t). One symbol corresponds to a level of confidence of P<0.05, two symbols to P<0.01, and three symbols to P<0.001.

FIG. 29 shows (A) total IgG; (B) IgG toward NP and (C) IgG response toward rVLP-SM (all on log scale) as measured by ELISA for macaques immunized twice (day 0 and 28) with a human dose (14 μg) of Fluviral® 2008 or Fluviral® 2008 adjuvanted with 150 μg rVLP-SM (*P<0.05 and ***P<0.001).

FIG. 30 shows (A) IgG2a titer directed to Fluviral® 2008 (log scale), and (B) IgG2a titer directed to NP (log scale), as measured by ELISA for Balb/C mice (10 per group) immunized with one subcutaneous injection of the Fluviral® 2008 vaccine alone (⅕ of a human dose, 3 μg), or Fluviral® 2008 adjuvanted with increasing amounts of rVLP-SM (30, 60 or 120 μg). **P<0.01 and P<0.0001.

FIG. 31 shows (A) IgG total titers directed to Fluviral® 2009 (log scale); (B) IgG2a titers directed to Fluviral® 2009; (C) IgG total titers directed to Influvac 2009 (log scale) and (D) IgG2a titers directed to Influvac® 2009, as measured by ELISA for Balb/C mice (10 per group) immunized twice at 14-day intervals with one subcutaneous injection of either the Fluviral® 2009 or the Influvac 2009 vaccine alone (⅕ of a human dose, 3 μg), or with one of the commercial vaccines adjuvanted with rVLP-SM 30 μg.

FIG. 32 presents (A) weight curve, (B) survival curve, and (C) symptoms, for mice vaccinated with Fluviral 2009, Influvac 2009 or the commercial vaccine adjuvanted with 30 μg of rVLP-SM (as for FIG. 31) and challenged with 1LD₅₀ of the heterologous influenza strain WSN/33.

FIG. 33 shows the total IgG (A) and IgG2a (B) to Fluviral 2009 and the IgG2a to purified GST-NP (C) as measured by ELISA for Balb/C mice (10 per group) immunized twice at 14-day intervals with one subcutaneous injection of Fluviral® 2009 (⅕ of a human dose, 3 μg) (one group), with the commercial vaccine adjuvanted with rVLP-SM 30 μg (3 groups) or with the adjuvant rVLP-SM alone 30 μg.

FIG. 34 presents (A) weight curve, (B) survival curve, and (C) symptoms, for mice vaccinated with Fluviral® 2009, with the commercial vaccine adjuvanted with rVLP-SM or with rVLP-SM alone (as described for FIG. 33) and challenged with 1LD₅₀ of the heterologous influenza strain WSN/33. Data were measured every day during 14 days.

FIG. 35 shows (A) IgG2a titers directed to Fluviral® 2008 and (B) IgG2a titers directed to NP protein at 2 months after immunization in Balb/C mice (10 per group) immunized once with one subcutaneous injection of the Fluviral® 2008 alone (⅕ of a human dose, 3 μg), or the commercial vaccine adjuvanted with rVLP-SM 30 μg.

FIG. 36 presents (A) the weight curve, (B) the survival curve, and (C) the symptoms of mice vaccinated with Fluviral 2008, or with the commercial vaccine adjuvanted with 30 μg of rVLP-SM (as described for FIG. 35) after challenge with 1LD₅₀ of the heterologous influenza strain WSN/33, where the challenge was performed ten months after the immunisation of the animals.

### DETAILED DESCRIPTION OF THE INVENTION

The present invention provides multivalent vaccine compositions that are based on the adjuvant properties of papaya mosaic virus. In one aspect the multivalent vaccine compositions provide protection against more than one strain of a pathogen and, in a specific embodiment, against more than one strain of the influenza virus. In another aspect, the multivalent vaccine compositions provide protection against more than one pathogen. The multivalent vaccine compositions comprise as core components, a papaya mosaic virus (PapMV) component and one or more antigens. The PapMV component can be PapMV or PapMV virus-like particles (VLPs). The multivalent vaccine compositions can optionally further comprise a Salmonella typhi porin component.

The one or more antigens can be combined with or conjugated to the PapMV component. The antigens can be derived from various pathogens against which it is desirable to provide protection. The antigens can be purified or partially purified and, in certain embodiments, can be provided in the form of a pre-formulated vaccine. In one embodiment, the antigens are derived from the influenza virus. In a specific embodiment, the antigens are provided in the form of a pre-formulated influenza vaccine, for example, a commercially available influenza vaccine.

In one embodiment of the invention, the multivalent vaccine composition comprises a PapMV component and one or more antigens in the form of an influenza vaccine. In a specific embodiment, the multivalent vaccine composition comprises PapMV VLPs and an influenza vaccine. In accordance with these embodiments, the multivalent vaccine composition provides protection against a plurality of influenza strains. In one embodiment, the PapMV component acts to extend the protection afforded by the vaccine to include protection against heterologous strains of influenza against which the vaccine alone typically does not provide adequate protection. In a further embodiment, a multivalent vaccine composition comprising PapMV VLPs and an influenza vaccine provides long-lasting protection against the influenza virus, for example, for at least 6 months after inoculation.

A specific embodiment of the invention provides for the use of PapMV VLPs made to improve the immunogenicity of a seasonal trivalent influenza vaccine by increasing the cellular and humoral immune responses in a subject to one or more highly conserved epitopes of the influenza virus. In accordance with this embodiment, the use of the PapMV VLPs in combination with an influenza vaccine leads to protection against heterologous strains of the influenza virus. A heterologous strain is defined in this context as a strain that is not present into the trivalent influenza vaccine.

In another embodiment of the invention, the multivalent vaccine compositions further comprise a Salmonella typhi porin component. In accordance with this embodiment, therefore, the multivalent vaccine compositions comprise a PapMV component, a S. typhi porin component and one or more antigens. The porin component can be a Salmonella spp. OmpC or OmpF, or a combination thereof, and can be combined with the PapMV component or conjugated to the PapMV component. For ease of reference, when a multivalent vaccine composition according to the present invention comprises both PapMV and porin components, the PapMV component and porin component are collectively referred to herein as “PapMV-porin.” In one embodiment, the multivalent vaccine composition comprising a PapMV-porin and one or more antigens provides protection against a plurality of strains of a pathogen, for example, an influenza virus. In another embodiment, the PapMV-porin in the multivalent vaccine composition functions as an adjuvant and/or an immunostimulant with respect to the one or more antigens, as well as to induce an immune response against S. typhi infection. In accordance with this embodiment, therefore, the multivalent vaccine composition comprising PapMV-porin and one or more antigens is capable of providing protection against more than one pathogen. A further aspect of the invention provides for the use of the PapMV-porin as an adjuvant and methods of potentiating an immune response by administering the PapMV-porin in combination with one or more antigens.

### DEFINITIONS

Unless defined otherwise, all technical and scientific terms used herein have the same meaning as commonly understood by one of ordinary skill in the art to which this invention belongs.

As used herein, the term “about” refers to approximately a+/−10% variation from a given value. It is to be understood that such a variation is always included in any given value provided herein, whether or not it is specifically referred to.

The term “adjuvant,” as used herein, refers to an agent that augments, stimulates, actuates, potentiates and/or modulates an immune response in an animal. An adjuvant may or may not have an effect on the immune response in itself.

The term “immune response,” as used herein, refers to an alteration in the reactivity of the immune system of an animal in response to an antigen or antigenic material and may involve antibody production, induction of cell-mediated immunity, complement activation, development of immunological tolerance, or a combination thereof.

The term “immunoprotective response,” as used herein, means an immune response that is directed against one or more antigen so as to protect against a condition (for example, a disease or disorder) and/or infection caused by an agent from which the one or more antigens are derived. For purposes of the present invention, immunoprotection against a condition and/or infection includes not only the absolute prevention of the condition or infection, but also any detectable reduction in the degree or rate of the condition or infection, or any detectable reduction in the severity of the condition or any symptom resulting from infection by the agent in a treated animal as compared to an untreated animal suffering from the condition or infection. An immunoprotective response can be induced in animals that were not previously suffering from the condition, have not previously been infected with the agent and/or do not have the condition or infection at the time of treatment. An immunoprotective response can also be induced in an animal already suffering from the condition or infected with the pathogen at the time of treatment. The immunoprotective response can be the result of one or more mechanisms, including humoral and/or cellular immunity.

The terms “immune stimulation” and “immunostimulation” as used interchangeably herein, refer to the ability of a molecule, such as a VLP, that is unrelated to an animal pathogen or disease to provide protection to against infection by the pathogen or against the disease by stimulating the immune system and/or improving the capacity of the immune system to respond to the infection or disease. Immunostimulation may have a prophylactic effect, a therapeutic effect, or a combination thereof.

A “recombinant virus” is one in which the genetic material of a naturally-occurring virus has combined with other genetic material.

“Naturally occurring,” as used herein, as applied to an object, refers to the fact that an object can be found in nature. For example, an organism (including a virus), or a polypeptide or polynucleotide sequence that is present in an organism that can be isolated from a source in nature and which has not been intentionally modified by man in the laboratory is naturally occurring.

The terms “polypeptide” or “peptide” as used herein is intended to mean a molecule in which there is at least four amino acids linked by peptide bonds.

The expression “viral nucleic acid,” as used herein, may be the genome (or a majority thereof) of a virus, or a nucleic acid molecule complementary in base sequence to that genome. A DNA molecule that is complementary to viral RNA is also considered viral nucleic acid, as is a RNA molecule that is complementary in base sequence to viral DNA.

The term “virus-like particle” (VLP), as used herein, refers to a self-assembling particle which has a similar physical appearance to a virus particle. The VLP may or may not comprise viral nucleic acids. VLPs are generally incapable of replication. In the context of the present invention, a PapMV VLP is a VLP derived from a PapMV coat protein. By “derived from” it is meant that the VLP comprises coat proteins that have an amino acid sequence substantially identical to the sequence of the wild-type PapMV coat protein and may optionally include one or more peptides attached to the coat protein, as described in more detail below. The PapMV coat protein included in the VLP can thus be the wild-type coat protein or a modified version thereof which is capable of multimerization and self-assembly to form a VLP.

The term “pseudovirus,” as used herein, refers to a VLP that comprises nucleic acid sequences, such as DNA or RNA, including nucleic acids in plasmid form. Pseudoviruses are generally incapable of replication.

The term “vaccine,” as used herein, refers to a material capable of producing an immunoprotective response in a subject.

The terms “immunogen” and “antigen” as used herein refer to a molecule, molecules, a portion or portions of a molecule, or a combination of molecules, up to and including whole cells and tissues, which are capable of inducing an immune response in a subject alone or in combination with an adjuvant. The immunogen/antigen may comprise a single epitope or may comprise a plurality of epitopes. The term thus encompasses, for example, peptides, carbohydrates, proteins, nucleic acids, and various microorganisms, in whole or in part, including viruses, bacteria and parasites. Haptens are also considered to be encompassed by the terms “immunogen” and “antigen” as used herein.

The terms “immunization” and “vaccination” are used interchangeably herein to refer to the administration of a vaccine to a subject for the purposes of raising an immune response and can have a prophylactic effect, a therapeutic effect, or a combination thereof. Immunization can be accomplished using various methods depending on the subject to be treated including, but not limited to, intraperitoneal injection (i.p.), intravenous injection (i.v.), intramuscular injection (i.m.), oral administration, intranasal administration, spray administration and immersion.

The term “prime” and grammatical variations thereof, as used herein, means to stimulate and/or actuate an immune response against an antigen in an animal prior to administering a booster vaccination with the antigen.

As used herein, the terms “treat,” “treated,” or “treating” when used with respect to a condition, such as a disease or disorder, or infectious agent refers to a treatment which increases the resistance of a subject to the condition or to infection with a pathogen (i.e. decreases the likelihood that the subject will contract the condition or become infected with the agent) as well as a treatment after the subject has contracted the condition or become infected in order to fight a condition or infection (for example, reduce, eliminate, ameliorate or stabilise a condition or infection, or symptoms associated therewith).

The term “subject” or “patient” as used herein refers to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep and other hoofed animals; dogs; cats; chickens; ducks; non-human primates; guinea pigs; rabbits; ferrets; rats; hamsters and mice.

The term “substantially identical,” as used herein in relation to a nucleic acid or amino acid sequence indicates that, when optimally aligned, for example using the methods described below, the nucleic acid or amino acid sequence shares at least 70%, at least 75%, at least 80%, at least 85%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The terms “corresponding to” or “corresponds to” indicate that a nucleic acid sequence is identical to all or a portion of a reference nucleic acid sequence. In contradistinction, the term “complementary to” is used herein to indicate that the nucleic acid sequence is identical to all or a portion of the complementary strand of a reference nucleic acid sequence. For illustration, the nucleic acid sequence “TATAC” corresponds to a reference sequence “TATAC” and is complementary to a reference sequence “GTATA.”

The term “plurality” as used herein means more than one, for example, two or more, three or more, four or more, and the like.

The term “heterologous strain” as used herein with reference to an influenza virus means a strain of influenza virus that is different to the strain(s) included in the vaccine administered to a subject.

### Multivalent Vaccine Compositions

A multivalent vaccine composition of the present invention comprises a PapMV component and one or more antigens, and optionally a porin component. The PapMV component can be PapMV or a VLP derived from PapMV coat protein. The PapMV coat protein can be the wild-type coat protein or a modified version thereof which is capable of multimerization and self-assembly to form a VLP.

In one embodiment, the multivalent vaccine further comprises a porin component, which can be for example OmpC, OmpF, or a combination thereof, where the OmpC or OmpF is substantially identical to OmpC or OmpF from the enterobacterium Salmonella enterica susp. enterica Serovar Typhi (S. typhi) as described in more detail below.

When the multivalent vaccine composition of the invention comprises both a PapMV component and a porin component, they are generally included in the PapMV-porin in a PapMV:porin ratio of between about 20:1 and about 1:10 by weight. In one embodiment of the invention, the PapMV-porin comprises the PapMV component and porin component in a ratio of between about 15:1 and about 1:10 by weight. In other embodiments of the invention, the PapMV-porin comprises the PapMV component and porin component in a ratio of between about 12:1 and about 1:10 by weight, between about 10:1 and about 1:10 by weight, between about 10:1 and about 1:5 by weight, between about 5:1 and about 1:5 by weight; between about 4:1 and about 1:4 by weight; between about 3:1 and about 1:3 by weight, and between about 2:1 and about 1:2 by weight. In one embodiment of the invention, the PapMV-porin comprises the PapMV component and porin component in a ratio of about 1:1 by weight.

The one or more antigens comprised by the multivalent vaccine composition can be conjugated to a coat protein of the PapMV or PapMV VLP, or they may be non-conjugated (i.e. separate from the PapMV or PapMV VLP).

Although the multivalent vaccines of the present invention are, for convenience, referred to herein by the term “multivalent vaccine compositions,” it is to be understood that this term includes situations in which the multivalent vaccine comprises separate formulations of the PapMV component and antigen(s) (for example, as a combination product). A non-limiting example of this kind of situation occurs when the PapMV component is combined with a commercial vaccine to provide a multivalent vaccine.

### PapMV Component

The PapMV component for inclusion in the multivalent vaccines in accordance with the present invention comprises either PapMV or PapMV VLPs. PapMV VLPs are formed from recombinant PapMV coat proteins that have multimerised and self-assembled to form a VLP. When assembled, each VLP comprises a long helical array of coat protein subunits. The wild-type virus comprises over 1200 coat protein subunits and is about 500 nm in length. PapMV VLPs that are either shorter or longer than the wild-type virus can still, however, be effective. In one embodiment of the present invention, the VLP comprises at least 20 coat protein subunits. In another embodiment, the VLP comprises between about 20 and about 1600 coat protein subunits. In an alternative embodiment, the VLP is at least 40 nm in length. In another embodiment, the VLP is between about 40 nm and about 600 nm in length.

The VLPs of the present invention can be prepared from a plurality of recombinant coat proteins having identical amino acid sequences, such that the final VLP when assembled comprises identical coat protein subunits, or the VLP can be prepared from a plurality of recombinant coat proteins having different amino acid sequences, such that the final VLP when assembled comprises variations in its coat protein subunits.

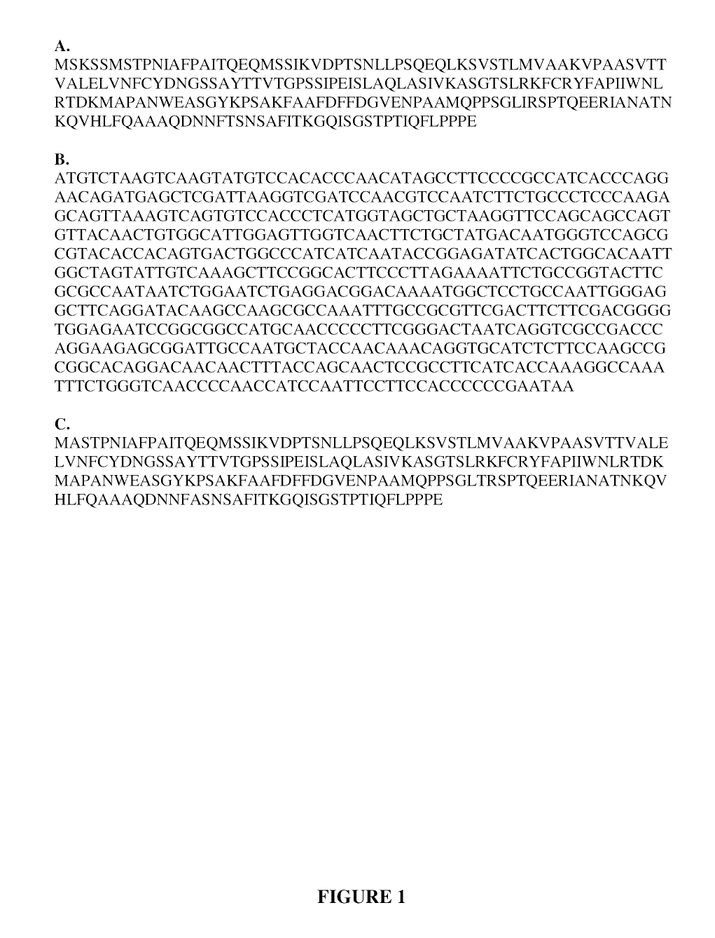

The coat protein used to form the VLP can be the entire PapMV coat protein, or part thereof, or it can be a genetically modified version of the PapMV coat protein, for example, comprising one or more amino acid deletions, insertions, replacements and the like, provided that the coat protein retains the ability to multimerise and assemble into a VLP. The amino acid sequence of the wild-type PapMV coat (or capsid) protein is known in the art (see, Sit, et al., 1989, J. Gen. Virol., 70:2325-2331, and GenBank Accession No. NP_{—}044334.1) and is provided herein as SEQ ID NO:1 (see FIG. 1A). The nucleotide sequence of the PapMV coat protein is also known in the art (see, Sit, et al., ibid., and GenBank Accession No. NC_{—}001748 (nucleotides 5889-6536)) and is provided herein as SEQ ID NO:2 (see FIG. 1B). In one embodiment of the invention, the PapMV coat protein is substantially identical to the wild-type PapMV coat protein as depicted in SEQ ID NO:1.

As noted above, the amino acid sequence of the recombinant PapMV coat protein comprised by the VLP need not correspond precisely to the parental sequence, i.e. it may be a modified or “variant sequence.” For example, the recombinant protein may be mutagenized by substitution, insertion or deletion of one or more amino acid residues so that the residue at that site does not correspond to the parental (reference) sequence. One skilled in the art will appreciate, however, that such mutations will not be extensive and will not dramatically affect the ability of the recombinant coat protein to multimerise and assemble into a VLP. The ability of a variant version of the PapMV coat protein to assemble into multimers and VLPs can be assessed, for example, by electron microscopy following standard techniques, such as the exemplary methods set out in the Examples provided herein. Various mutations that are tolerated by the PapMV coat protein while not affecting its ability to form VLPs are known in the art (see, for example, Tremblay, M-H., et al., 2006, FEBS J., 273:14-25, and Lecours et al., 2006, Protein Expression and Purification, 47:273-280).

Recombinant coat proteins that are fragments of the wild-type protein that retain the ability to multimerise and assemble into a VLP (i.e. are “functional” fragments) are, therefore, also contemplated by the present invention. For example, a fragment may comprise a deletion of one or more amino acids from the N-terminus, the C-terminus, or the interior of the protein, or a combination thereof. In general, functional fragments are at least 100 amino acids in length. In one embodiment of the present invention, functional fragments are at least 150 amino acids, at least 160 amino acids, at least 170 amino acids, at least 180 amino acids, and at least 190 amino acids in length. Deletions made at the N-terminus of the protein should generally delete fewer than 25 amino acids in order to retain the ability of the protein to multimerise.

In accordance with the present invention, when a recombinant coat protein comprises a variant sequence, the variant sequence is at least about 70% identical to the reference sequence. In one embodiment, the variant sequence is at least about 75% identical to the reference sequence. In other embodiments, the variant sequence is at least about 80%, at least about 85%, at least about 90%, at least about 95%, and at least about 97% identical to the reference sequence. In a specific embodiment, the reference amino acid sequence is SEQ ID NO:1.

In one embodiment of the present invention, the VLP comprises a genetically modified (i.e. variant) version of the PapMV coat protein. In another embodiment, the PapMV coat protein has been genetically modified to delete amino acids from the N- or C-terminus of the protein and/or to include one or more amino acid substitutions. In a further embodiment, the PapMV coat protein has been genetically modified to delete between about 1 and about 10 amino acids from the N- or C-terminus of the protein.

In a specific embodiment, the PapMV coat protein has been genetically modified to remove one of the two methionine codons that occur proximal to the N-terminus of the protein (i.e. at positions 1 and 6 of SEQ ID NO:1) and can initiate translation. Removal of one of the translation initiation codons allows a homogeneous population of proteins to be produced. The selected methionine codon can be removed, for example, by substituting one or more of the nucleotides that make up the codon such that the codon codes for an amino acid other than methionine, or becomes a nonsense codon. Alternatively all or part of the codon, or the 5′ region of the nucleic acid encoding the protein that includes the selected codon, can be deleted. In a specific embodiment of the present invention, the PapMV coat protein has been genetically modified to delete between 1 and 5 amino acids from the N-terminus of the protein. In a further embodiment, the genetically modified PapMV coat protein has an amino acid sequence substantially identical to the sequence as set forth in SEQ ID NO:3 (FIG. 1C).

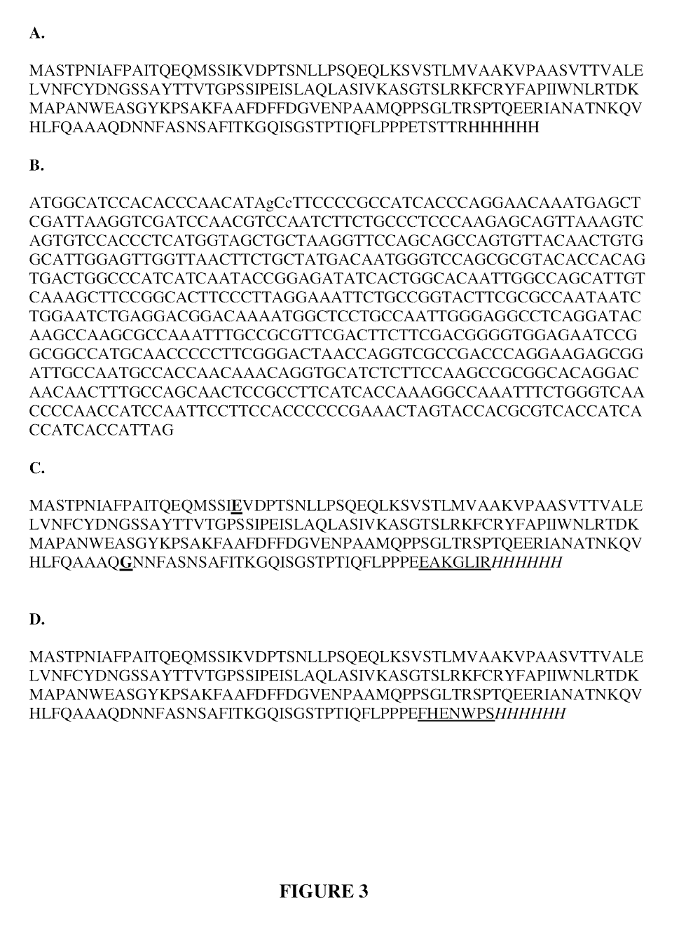

In a further embodiment, the genetically modified coat protein is substantially identical to the sequence as set forth in SEQ ID NO:37 (see FIG. 3A).

When the recombinant coat protein comprises a variant sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

In one embodiment of the present invention, the variant sequence comprises one or more non-conservative substitutions. Replacement of one amino acid with another having different properties may improve the properties of the coat protein. For example, as described in Tremblay, M-H., et al. (2006, FEBS J., 273:14-25), mutation of residue 128 of the coat protein can improve assembly of the protein into VLPs. In one embodiment of the present invention, therefore, the coat protein comprises a mutation at residue 128 of the coat protein in which the glutamic residue at this position is substituted with a neutral residue. In a further embodiment, the glutamic residue at position 128 is substituted with an alanine residue.

Substitution of the phenylalanine residue at position F13 of the PapMV coat protein with another hydrophobic residue has been shown to result in a higher proportion of VLPs being formed when the recombinant protein is expressed than when the wild-type protein sequence is used. In the context of the present invention, the following amino acid residues are considered to be hydrophobic residues suitable for substitution at the F13 position: Ile, Trp, Leu, Val, Met and Tyr. In one embodiment of the invention, the coat protein comprises a substitution of Phe at position 13 with Ile, Trp, Leu, Val, Met or Tyr. In another embodiment, the coat protein comprises a substitution of Phe at position 13 with Leu or Tyr.

Likewise, the nucleic acid sequence encoding the recombinant coat protein need not correspond precisely to the parental reference sequence but may vary by virtue of the degeneracy of the genetic code and/or such that it encodes a variant amino acid sequence as described above. In one embodiment of the present invention, therefore, the nucleic acid sequence encoding the recombinant coat protein is at least about 70% identical to the reference sequence. In another embodiment, the nucleic acid sequence encoding the recombinant coat protein is at least about 75% identical to the reference sequence. In other embodiments, the nucleic acid sequence encoding the recombinant coat protein is at least about 80%, at least about 85% or at least about 90% identical to the reference sequence. In a specific embodiment, the reference nucleic acid sequence is SEQ ID NO:2.

The PapMV VLP coat protein may optionally be genetically fused to an affinity peptide or other short peptide sequence to facilitate attachment of the OmpC or OmpF component, as described in more detail below.

### Preparation of the PapMV Component

### PapMV

PapMV is known in the art and can be obtained, for example, from the American Type Culture Collection (ATCC) as ATCC No. PV-204TH. The virus can be maintained on, and purified from, host plants such as papaya (Carica papaya) and snapdragon (Antirrhinum majus) following standard protocols (see, for example, Erickson, J. W. & Bancroft, J. B., 1978, Virology 90:36-46).

### PapMV VLPs

Recombinant PapMV coat proteins to be used to prepare PapMV VLPs can be readily prepared by standard genetic engineering techniques by the skilled worker provided with the sequence of the wild-type protein. Methods of genetically engineering proteins are well known in the art (see, for example, Ausubel et al. (1994 & updates) Current Protocols in Molecular Biology, John Wiley & Sons, New York), as is the sequence of the wild-type PapMV coat protein (see SEQ ID NOs:1 and 2).

Isolation and cloning of the nucleic acid sequence encoding the wild-type protein can be achieved using standard techniques (see, for example, Ausubel et al., ibid.). For example, the nucleic acid sequence can be obtained directly from the PapMV by extracting RNA by standard techniques and then synthesizing cDNA from the RNA template (for example, by RT-PCR). PapMV can be purified from infected plant leaves that show mosaic symptoms by standard techniques.

The nucleic acid sequence encoding the coat protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described in more detail below.

Alternatively, the nucleic acid sequence encoding the coat protein can be further engineered to introduce one or more mutations, such as those described above, by standard in vitro site-directed mutagenesis techniques well-known in the art.

Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

As described in more detail below, the coat proteins can also be engineered to produce fusion proteins comprising one or more affinity peptides fused to the coat protein. Methods for making fusion proteins are well known to those skilled in the art. DNA sequences encoding a fusion protein can be inserted into a suitable expression vector as noted above.

One of ordinary skill in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the coat or fusion protein. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered coat protein. One skilled in the art will appreciate that selection of suitable regulatory elements is dependent on the host cell chosen for expression of the genetically engineered coat protein and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids encoded by the heterologous nucleic acid sequence can be removed from the expressed coat protein prior to use according to methods known in the art. Alternatively, the amino acids corresponding to expression of heterologous nucleic acid sequences can be retained on the coat protein if they do not interfere with its subsequent assembly into VLPs.

In one embodiment of the present invention, the coat protein is expressed as a histidine tagged protein. The histidine tag can be located at the carboxyl terminus or the amino terminus of the coat protein.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (e.g. E. coli, A. salmonicida or B. subtilis) or in a eukaryotic host (e.g. Saccharomyces or Pichia; mammalian cells, e.g. COS, NIH 3T3, CHO, BHK, 293 or HeLa cells; insect cells or plant cells).

If desired, the coat proteins can be purified from the host cells by standard techniques known in the art (see, for example, in Current Protocols in Protein Science, ed. Coligan, J. E., et al., Wiley & Sons, New York, N.Y.) and sequenced by standard peptide sequencing techniques using either the intact protein or proteolytic fragments thereof to confirm the identity of the protein.

The recombinant coat proteins of the present invention are capable of multimerisation and assembly into VLPs. Assembly of the VLPs can take place in the host cell expressing the coat protein and the VLPs can be isolated from the host cells by standard techniques, such as those described in the Examples section provided herein. The VLPs can be further purified by standard techniques, such as chromatography, to remove contaminating host cell proteins or other compounds, such as LPS. In one embodiment of the present invention, the VLPs are purified to remove LPS.

### Characteristics of Recombinant PapMV Coat Proteins

Recombinant coat proteins, including coat proteins to which affinity peptides have been fused (see below) can be analysed for their ability to multimerize and self-assemble into VLPs by standard techniques. For example, by visualising the purified protein by electron microscopy (see, for example, Tremblay, M-H., et al., 2006, FEBS J., 273:14-25). In addition, ultracentrifugation may be used to isolate VLPs as a pellet, while leaving smaller aggregates (20-mers and less) in the supernatant, and circular dichroism (CD) spectrophotometry may be used to compare the secondary structure of the recombinant or modified proteins with the WT virus (see, for example, Tremblay et al., ibid.).

Stability of the VLPs and of PapMV can be determined if desired by techniques known in the art, for example, by SDS-PAGE and proteinase K degradation analyses. According to one embodiment of the present invention, the PapMV component utilised in the nultivalent vaccine compositions is stable at elevated temperatures and can be stored easily at room temperature.

### Antigens

As noted above, the multivalent vaccine compositions of the invention include one or more antigens. The antigens can be purified or partially purified, for example, antigenic proteins or protein fragments, or whole cells or fragments of whole cells. Alternatively, the one or more antigens can be provided in the form of a pre-formulated and/or commercially available vaccine.

A wide variety of antigens suitable for the development of vaccines is known in the art. Appropriate antigens for inclusion in the multivalent vaccine compositions of the invention can be readily selected by one skilled in the art based on, for example, the desired end use of the vaccine, such as the diseases or disorders against which it is to be directed, the format of composition, and/or the animal to which it is to be administered.

For example, the antigens can be derived from an agent capable of causing a disease or disorder in an animal, such as a cancer, infectious disease, allergic reaction, or autoimmune disease, or they can be antigens suitable for use to induce an immune response against drugs, hormones or a toxin-associated disease or disorder. The antigens may be derived from a known pathogen, such as, for example, a bacterium, virus, protozoan, fungus, parasite, or infectious particle, such as a prion, or the antigens may be tumour-associated antigens, self-antigens or allergens.

In one embodiment of the invention, the antigens are derived from an agent that causes a disease or disorder in the infected subject. By way of example, the antigens may be derived from known causative agents responsible for diseases such as Diptheria (e.g. Corynebacterium diphtheriae), Pertussis (e.g. Bordetella pertussis), Tetanus (e.g. Clostridium tetani), Tuberculosis (e.g. Mycobacterium tuberculosis), Bacterial or Fungal Pneumonia, Cholera (e.g. Vibrio cholerae), Typhoid fever (e.g. S. typhi), Plague, Shigellosis (e.g. Shigella dysenteriae serotype 1 (S. dysenteriae 1)), Salmonellosis, Legionnaire's Disease (e.g. Legionella pneumophila), Lyme Disease, Leprosy (e.g. Mycobacterium leprae), Malaria (e.g. Plasmodium falciparium), Hookworm, Onchocerciasis, Schistosomiasis, Trypamasomialsis, Leshmaniasis, Giardia (e.g. Giardia lamblia), Amoebiasis (e.g. Entamoeba histolytica), Filariasis, Borrelia, Trichinosis, Influenza, hepatitis B and C, Meningococcal meningitis, Community Acquired Pneumonia, Chickenpox, Rubella, Mumps, Diphtheria, Measles, AIDS, Dengue Respiratory infections, Diarrhoeal Diseases, Tropical parasitic diseases, sexually transmitted diseases and Chlamydia infections. Antigens for inclusion in the multivalent vaccine compositions may also be derived from causative agents responsible for new emerging, re-emerging diseases or bioterrorism diseases such as: SARS infection, Vancomycin-resistant S. aureus infections, West Nile Virus infections, Cryptosporidiosis, Hanta virus infections, Epstein Barr Virus infections, Cytomegalovirus infections, H5N1 Influenza, Enterovirus 71 infections, E. coli O157:H7 infections, Human Monkey pox, Lyme disease, Cyclosporiasis, Hendra virus infections, Nipah virus infections, Rift Valley fever, Marburg haemorrhagic fever, Whitewater arrollo virus infections and Anthrax.

The size of the antigen(s) for incorporation into the multivalent vaccine compositions is not critical to the invention and the selected antigen(s) can thus vary in size. The antigen(s) may be, for example, peptide, protein, nucleic acid, polysaccharide, lipid, or small molecule antigens, or a combination thereof, up to and including a whole pathogen or a portion thereof, for example, a live, inactivated or attenuated version of a pathogen.

When more than one antigen is selected for incorporation into the multivalent vaccine compositions of the invention, the antigens selected for inclusion in the product can be derived from a single source, or can be derived from a plurality of sources. The antigens can each have a single epitope capable of triggering a specific immune response, or each antigen may comprise more than one epitope.

The antigen(s) may comprise epitopes recognised by surface structures on T cells, B cells, NK cells, dendritic cells, macrophages, polymorphonuclear leukocytes, Class I or Class II APC associated cell surface structures, or a combination thereof.

Antigens for inclusion in the multivalent vaccine compositions of the invention may also be selected from pathogens or other sources of interest by art known methods and screened for their ability to induce an immune response in an animal using standard immunological techniques known in the art. For example, methods for prediction of epitopes within an antigenic protein are described in Nussinov R and Wolfson H J, Comb Chem High Throughput Screen (1999) 2(5):261, and methods of predicting CTL epitopes are described in Rothbard et al., EMBO J. (1988) 7:93-100 and in de Groot M S et al., Vaccine (2001) 19(31):4385-95. Other methods are described in Rammensee H-G. et al., Immunogenetics (1995) 41:178-228 and Schirle Metal., Eur J Immunol (2000) 30(18):2216-2225.

Useful viral antigens include, for example, antigens derived from members of the families Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxyiridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). In one embodiment, the multivalent vaccine composition comprises one or more antigens derived from a major viral pathogen such as the various hepatitis viruses, polio virus, human immunodeficiency virus (HIV), various influenza viruses, West Nile virus, respiratory syncytial virus, rabies virus, human papilloma virus (HPV), Epstein Barr virus (EBV), polyoma virus, or SARS coronavirus.

Antigens derived from the hepatitis viruses, including hepatitis A virus (HAV), hepatitis B virus (HBV), hepatitis C virus (HCV), the delta hepatitis virus (HDV), hepatitis E virus (HEV) and hepatitis G virus (HGV), are known in the art. For example, antigens can be derived from HCV core protein, E1 protein, E2 protein, NS3 protein, NS4 protein or NS5 protein, from HBV HbsAg antigen or HBV core antigen, and from HDV delta-antigen (see, for example, U.S. Pat. No. 5,378,814). U.S. Pat. Nos. 6,596,476; 6,592,871; 6,183,949; 6,235,284; 6,780,967; 5,981,286; 5,910,404; 6,613,530; 6,709,828; 6,667,387; 6,007,982; 6,165,730; 6,649,735 and 6,576,417, for example, describe various antigens based on HCV core protein.

Non-limiting examples of known antigens from the herpesvirus family include those derived from herpes simplex virus (HSV) types 1 and 2, such as HSV-1 and HSV-2 glycoproteins gB, gD and gH.

Non-limiting examples of HIV antigens include antigens derived from gp120, antigens derived from various envelope proteins such as gp160 and gp41, gag antigens such as p24gag and p55gag, as well as proteins derived from the pol, env, tat, vif rev, nef vpr, vpu and LTR regions of HIV. The sequences of gp120 from a multitude of HIV-1 and HIV-2 isolates, including members of the various genetic subtypes of HIV are known (see, for example, Myers et al., Los Alamos Database, Los Alamos National Laboratory, Los Alamos, N. Mex. (1992); and Modrow et al., J. Virol. (1987) 61:570 578).

Non-limiting examples of other viral antigens include antigens from varicella zoster virus (VZV), Epstein-Barr virus (EBV) and cytomegalovirus (CMV) including CMV gB and gH; and antigens from other human herpesviruses such as HHV6 and HHV7 (see, for example Chee et al. (1990) Cytomegaloviruses (J. K. McDougall, ed., Springer-Verlag, pp. 125-169; McGeoch et al. (1988) J. Gen. Virol. 69:1531-1574; U.S. Pat. No. 5,171,568; Baer et al. (1984) Nature 310:207-211; and Davison et al. (1986) J. Gen. Virol. 67:1759-1816).

Antigens for inclusion in the multivalent vaccine compositions can also be derived from the influenza virus, for example, the antigenic material can be attenuated, killed or inactivated influenza virus. Alternatively, the antigenic material from the influenza virus can be derived from the haemagglutinin (HA), neuramidase (NA), nucleoprotein (NP), M1 or M2 proteins. The sequences of these proteins are known in the art and are readily accessible from GenBank database maintained by the National Center for Biotechnology Information (NCBI). Suitable antigenic fragments of HA, NP and the matrix proteins include, but are not limited to, the haemagglutinin epitopes: HA 91-108, HA 307-319 and HA 306-324 (Rothbard, Cell, 1988, 52:515-523), HA 458-467 (J. Immunol. 1997, 159(10): 4753-61), HA 213-227, HA 241-255, HA 529-543 and HA 533-547 (Gao, W. et al., J. Virol., 2006, 80:1959-1964); the nucleoprotein epitopes: NP 206-229 (Brett, 1991, J. Immunol. 147:984-991), NP335-350 and NP380-393 (Dyer and Middleton, 1993, In: Histocompatibility testing, a practical approach (Ed.: Rickwood, D. and Hames, B. D.) IRL Press, Oxford, p. 292; Gulukota and DeLisi, 1996, Genetic Analysis: Biomolecular Engineering, 13:81), NP 305-313 (DiBrino, 1993, PNAS 90:1508-12); NP 384-394 (Kvist, 1991, Nature 348:446-448); NP 89-101 (Cerundolo, 1991, Proc. R. Soc. Lon. 244:169-7); NP 91-99 (Silver et al, 1993, Nature 360: 367-369); NP 380-388 (Suhrbier, 1993, J. Immunology 79:171-173); NP 44-52 and NP 265-273 (DiBrino, 1993, ibid.); and NP 365-380 (Townsend, 1986, Cell 44:959-968); the matrix protein (M1) epitopes: M1 2-22, M1 2-12, M1 3-11, M13-12, M141-51, M150-59, M151-59, M1134-142, M1145-155, M1164-172, M1 164-173 (all described by Nijman, 1993, Eur. J. Immunol. 23:1215-1219); M117-31, M1 55-73, M1 57-68 (Carreno, 1992, Mol Immunol 29:1131-1140); M1 27-35, M1 232-240 (DiBrino, 1993, ibid.), M1 59-68 and M160-68 (Eur. J. Immunol. 1994, 24(3): 777-80); and M1 128-135 (Eur. J. Immunol. 1996, 26(2): 335-39).

Other related antigenic regions and epitopes of the influenza virus are also known. For example, fragments of the influenza ion channel protein (M2), including the M2e peptide (the extracellular domain of M2). The sequence of this peptide is highly conserved across different strains of influenza. An example of a M2e peptide sequence is shown in Table 1 as SEQ ID NO:16. Variants of this sequence have been identified and non-limiting examples are also shown in Table 1.

TABLE 1
M2e Peptide and Variations Thereof

| Region | | | SEQ ID |
|---|---|---|---|
| of M2 | Sequence | Viral Strain | NO |
| 2-24 | SLLTEVETPIRN | Human H1N1 e.g. | 16 |
| | EWGCRCNDSSD | A/USRR/90/77 and | |
| | | A/WSN/33 | |
| 2-24 | SLLTEVETPIRN | N/A* | 17 |
| | EWGCRCNGSSD | | |
| 2-24 | SLLTEVETPTKN | N/A* | 18 |
| | EWDCRCNDSSD | | |
| 2-24 | SLLTEVETPTRN | Equine H3N8 | 19 |
| | GWECKCSDSSD | A/equine/Massachussetts/ | |
| | | 213/2003 | |
| 2-24 | SLLTEVETPTRN | H5N1 A/Vietnam/1196/04 | 20 |
| | EWECRCSDSSD | | |
| 1-24 | MSLLTEVETPIR | Human H1N1 e.g. | 21 |
| | NEWGCRCNDSSD | A/USRR/90/77 and | |
| | | A/WSN/33 | |
| 1-24 | HSLLTEVETPTR | Avian H5N1 | 22 |
| | NEWECRCSDSSD | A/Vietnam/1196/04 | |
| 1-24 | MSLLTEVETPTR | H3N8, Horse-Dog | 23 |
| | NGWECKCSDSSD | A/equine/Massachussetts/ | |
| | | 213/2003 | |
| 1-24 | MSLLTEVETPTR | H9N2, | 24 |
| | NGWGCRCSDSSD | A/chicken/Osaka/aq69/ | |
| | | 2001 | |
| 1-24 | MSLLTEVETPTR | Mutant H1N1 I/T | 25 |
| | NEWGCRCSDSSD | | |

see U.S. Patent Application No. 2006/0246092

The entire M2e sequence or a partial M2e sequence may be used, for example, a partial sequence that is conserved across the variants, such as fragments within the region defined by amino acids 2 to 10, or the conserved epitope EVETPIRN [SEQ ID NO:26] (amino acids 6-13 of the M2e sequence). The 6-13 epitope has been found to be invariable in 84% of human influenza A strains available in GenBank. Variants of this sequence that were also identified include EVETLTRN [SEQ ID NO:27] (9.6%), EVETPIRS [SEQ ID NO:28] (2.3%), EVETPTRN [SEQ ID NO:29] (1.1%), EVETPTKN [SEQ ID NO:30] (1.1%) and EVDTLTRN [SEQ ID NO:31], EVETPIRK [SEQ ID NO:32] and EVETLTKN [SEQ ID NO:33] (0.6% each) (see Zou, P., et al., 2005, Int Immunopharmacology, 5:631-635; Liu et al. 2005, Microbes and Infection, 7:171-177).

As is known in the art, there are three genera of influenza virus: types A, B and C. Antigens for incorporation into the multivalent vaccine compositions of the invention may be derived from influenza virus type A, type B or type C, or a combination thereof. In one embodiment, the antigenic material for incorporation into the multivalent vaccine compositions of the invention is derived from influenza virus type A or type B, or a combination thereof. In addition, many strains of influenza are presently in existence. Important examples include, but are not limited to, those listed in Table 1. Antigens for incorporation into the multivalent vaccine compositions of the invention may be derived from one strain of influenza virus or multiple strains, for example, between two and five strains, in order to provide a broader spectrum of protection. In one embodiment, antigens for incorporation into the multivalent vaccine compositions of the invention are derived from multiple strains of influenza virus.

Other useful antigens include live, attenuated and inactivated viruses such as inactivated polio virus (Jiang et al., J. Biol. Stand., (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNE™ acellular DTP, Wyeth-Lederle Vaccines and Pediatrics).

Antigens can also be derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease, as are known in the art.

Useful bacterial antigens include, for example, whole inactivated cells, superficial bacterial antigenic components, such as lipopolysaccharides, capsular antigens (proteinacious or polysaccharide in nature), or flagellar components.

Examples of antigens derived from gram-negative bacteria of the family Enterobacteriaceae include, but are not limited to, the S. typhi Vi (capsular polysaccharide) antigen, the E. coli K and CFA (capsular component) antigens and the E. coli fimbrial adhesin antigens (K88 and K99). Examples of antigenic proteins include the outer membrane proteins related to OmpC and OmpF porins such as the S. typhi iron-regulated outer membrane protein (IROMP, Sood et al., 2005, Mol Cell Biochem 273:69-78), and heat shock proteins (HSPs) including, but not limited to S. typhi HSP40 (Sagi et al., 2006, Vaccine 24:7135-7141). Non-limiting examples of antigenic porins include non-Salmonella OmpC and OmpF, which are found in numerous Escherichia species. Orthologues of OmpC and OmpF are also found in other Enterobacteriaceae and are suitable antigenic proteins for the purposes of the present invention. In addition, Omp1B (Shigella flexneri), OmpC2 (Yersinia pestis), OmpD (S. enterica), OmpK36 (Klebsiella pneumoniae), OmpN (E. coli) and OmpS (S. enterica) may be suitable, based on conserved regions of sequences found in the porin proteins of the Enterobacteriaceae family (Diaz-Quinonez et al., 2004, Infect. and Immunity 72:3059-3062).

The sequences of antigenic proteins from various enterobacteria are known in the art and are readily accessible from GenBank database maintained by the National Center for Biotechnology Information (NCBI). For example, GenBank Accession No. 26248604: OmpC (E. coli); GenBank Accession No. 24113600: Omp1B (Shigella flexneri); GenBank Accession No. 16764875: OmpC2 (Yersinia pestis); GenBank Accession No. 16764916: OmpD (S. enterica Serovar Typhimurium); GenBank Accession No. 151149831: OmpK36 (Klebsiella pneumonie); GenBank Accession No. 3273514: OmpN (E. coli), and GenBank Accession No. 16760442: OmpS (S. enterica serovar Typhi). Toxins that can be used as antigens in the multivalent vaccine compositions of the invention are generally the natural products of toxic plants, animals, and microorganisms, or fragments of these compounds. Such compounds include, for example, aflatoxin, ciguautera toxin, pertussis toxin and tetrodotoxin. Other suitable toxins are known in the art.

Various tumour-associated antigens are known in the art. Representative examples include, but are not limited to, Her2 (breast cancer); GD2 (neuroblastoma); EGF-R (malignant glioblastoma); CEA (medullary thyroid cancer); CD52 (leukemia); human melanoma protein gp100; human melanoma protein melan-A/MART-1; human Dickkopf1 (DKK1) protein, human angiomotin (Amot), NA17; NA17-A nt protein; p53 protein; various MAGEs (melanoma associated antigen E), including MAGE 1, MAGE 2, MAGE 3 (HLA-A1 peptide) and MAGE 4; various tyrosinases (HLA-A2 peptide); mutant ras; p97 melanoma antigen; Ras peptide and p53 peptide associated with advanced cancers; the HPV 16/18 and E6/E7 antigens associated with cervical cancers; MUC1-KLH antigen associated with breast carcinoma; CEA (carcinoembryonic antigen) associated with colorectal cancer, and the PSA antigen associated with prostate cancer.

Examples of allergens include, but are not limited to, allergens from pollens, animal dander, grasses, moulds, dusts, antibiotics, stinging insect venoms, as well as a variety of environmental, drug and food allergens. Common tree allergens include pollens from cottonwood, popular, ash, birch, maple, oak, elm, hickory, and pecan trees. Common plant allergens include those from rye, ragweed, English plantain, sorrel-dock and pigweed, and plant contact allergens include those from poison oak, poison ivy and nettles. Common grass allergens include Timothy, Johnson, Bermuda, fescue and bluegrass allergens. Common allergens can also be obtained from moulds or fungi such as Alternaria, Fusarium, Hormodendrum, Aspergillus, Micropolyspora, Mucor and thermophilic actinomycetes. Penicillin, sulfonamides and tetracycline are common antibiotic allergens. Epidermal allergens can be obtained from house or organic dusts (typically fungal in origin), from insects such as house mites (Dermalphagoides pterosinyssis), or from animal sources such as feathers, and cat and dog dander. Common food allergens include milk and cheese (dairy), egg, wheat, nut (for example, peanut), seafood (for example, shellfish), pea, bean and gluten allergens. Common drug allergens include local anesthetic and salicylate allergens, and common insect allergens include bee, hornet, wasp and ant venom, and cockroach calyx allergens.

Particularly well characterized allergens include, but are not limited to, the dust mite allergens Der pI and Der pII (see, Chua, et al., J. Exp. Med., 167:175 182, 1988; and, Chua, et al., Int. Arch. Allergy Appl. Immunol., (1990) 91:124-129), T cell epitope peptides of the Der pII allergen (see, Joost van Neerven, et al., J. Immunol., (1993) 151:2326-2335), the highly abundant Antigen E (Amb aI) ragweed pollen allergen (see, Rafnar, et al., J. Biol. Chem., (1991) 266:1229-1236), phospholipase A2 (bee venom) allergen and T cell epitopes therein (see, Dhillon, et al., J. Allergy Clin. Immunol., (1992) 42), white birch pollen (Betyl) (see, Breiteneder, et al., EMBO, (1989) 8:1935-1938), the Fel dI major domestic cat allergen (see, Rogers, et al., Mol. Immunol., (1993) 30:559-568), tree pollen (see, Elsayed et al., Scand. J. Clin. Lab. Invest. Suppl., (1991) 204:17-31) and the multi-epitopic recombinant grass allergen rKBG8.3 (Cao et al. Immunology (1997) 90:46-51). These and other suitable allergens are commercially available and/or can be readily prepared following, known techniques.

Antigens relating to conditions associated with self antigens are also known to those of ordinary skill in the art. Representative examples of such antigens includes, but are not limited to, lymphotoxins, lymphotoxin receptors, receptor activator of nuclear factor kB ligand (RANKL), vascular endothelial growth factor (VEGF), vascular endothelial growth factor receptor (VEGF-R), interleukin-5, interleukin-17, interleukin-13, CCL21, CXCL12, SDF-1, MCP-1, endoglin, resistin, GHRH, LHRH, TRH, MIF, eotaxin, bradykinin, BLC, tumour Necrosis Factor alpha and amyloid beta peptide, as well as fragments of each which can be used to elicit immunological responses.

Antigens useful in relation to recreational drug addiction are known in the art and include, for example, opioids and morphine derivatives such as codeine, fentanyl, heroin, morphine and opium; stimulants such as amphetamine, cocaine, MDMA (methylenedioxymethamphetamine), methamphetamine, methylphenidate, and nicotine; hallucinogens such as LSD, mescaline and psilocybin; cannabinoids such as hashish and marijuana, other addictive drugs or compounds, and derivatives, by-products, variants and complexes of such compounds.

As noted above, in various embodiments of the invention, the one or more antigens included in the multivalent vaccine compositions of the invention can be in the form of a pre-formulated vaccine. Various human vaccines are known in the art and include, but are not limited to, vaccines against:
Bacillus anthracis (anthrax), such as BioThrax® (BioPort Corporation);
Haemophilus influenzae type b (Hib), such as, ActHIB® (Sanofi-aventis), PedvaxHIB® (Merck) and HibTITER® (Wyeth);
hepatitis A, such as, Havrix® (GlaxoSmithKline) and Vaqta® (Merck);
hepatitis B, such as, Engerix-B® (GlaxoSmithKline) and Recombivax HB®(Merck);
Herpes zoster (shingles), such as, Zostavax® (Merck);
human papillomavirus (HPV), such as, Gardasil® (Merck);
influenza, such as, Fluarix® and Fluviral® (GlaxoSmithKline), FluLaval®(ID Biomedical Corp of Quebec); FluMist® (intranasal) (Medimmune), Fluvirin® (Chiron), Influvac™ (Solvay) and Fluzone® (Sanofi-aventis);
Japanese encephalitis, such as, JE-Vax® (Sanofi-aventis);
measles, such as, Attenuvax® (Merck);
Meningococcal meninigitis, such as, Menomune® Meningococcal Polysaccharide (Sanofi-aventis);
mumps, such as, Mumpsvax® (Merck);
pneumococcal disease, such as, Pneumovax 23® Pneumococcal Polysaccharide (Sanofi-aventis) and Prevnar® Pneumococcal Conjugate (Wyeth);
polio, such as, Ipol® (Sanofi-aventis) and Poliovax® (Sanofi-Pasteur);
rabies, such as, BioRab® (BioPort Corporation), RabAvert® (Chiron) and Imovax® Rabies (Sanofi-aventis);
rotavirus, such as, RotaTeq® (Merck);
rubella, such as, Meruvax II® (Merck);
S. typhi (typhoid fever), such as, Typhim Vi® (Sanofi-aventis) and Vivotif®Berna (oral) (Berna);
tuberculosis (BCG), such as, TheraCys® and ImmuCyst® (Sanofi-aventis); TICE® BCG and Oncoticemi (Organon Teknika Corporation); Pacismi; and Mycobax® (Sanofi-Pasteur);
vaccinia (smallpox), such as, Dryvax® (Wyeth);
varicella (chickenpox), such as, Varivax® (Merck);
yellow fever, such as, YF-Vax® (Sanofi-aventis);
hepatitis A/hepatitis B, such as, Twinrix® (GlaxoSmithKline);
hepatitis B and Hib, such as, Comvax® (Merck);
tetanus/Hib, such as, ActHIB® (Sanofi-Pasteur);
diphtheria/Hib, such as, HibTITER® (Wyeth Pharmaceuticals);
Hib/meningitis, such as, PedVaxHIB (Merck & Co);
meningitis/diptheria, such as, Menactra® Meningococcal Conjugate (Sanofi-Pasteur);
tetanus/dipheria (Td), such as, Decavac® (Sanofi-aventis);
diphtheria/tetanus/pertussis (DTaP/DT or DTaP), such as, Daptacel® and Tripedia® (Sanofi-aventis) and Infanrix® (GlaxoSmithKline);
tetanus/diphtheria/pertussis (Tdap), such as, Boostrix® (GlaxoSmithKline) and Adacel® (Sanofi-Pasteur);
DTaP/Hib, such as, TriHIBit® (Sanofi-aventis);
DTaP/polio/hepatitis B, such as Pediarix® (GlaxoSmithKline);
measles/mumps/rubella (MMR), such as, M-M-R II (Merck) and
measles/mumps/rubella/chickenpox, such as, ProQuad® (Merck).

Examples of vaccines for veterinarian use include, but are not limited to, vaccines against Lawsonia intracellularis (for example, Enterisol and Ileitis), Porphyromonas gulae, and P. denticanis (for example, Periovac), Streptococcus equi (for example, Equilis StrepE), Chlamydophila abortus (for example, Ovilis and Enzovax), Mycoplasma synoviae (for example, Vaxsafe MS), Mycoplasma gallisepticum (for example, Vaxsafe MG), Bordetella avium (for example, Art Vax), Actinobacillus pleuropneumoniae (for example, PleuroStar APP), Actinobacillus pleuropneumoniae (for example, Porcilis APP), Salmonella (for example, Megan Vac1 and MeganEgg), Brucella abortus (for example, RB-51), Eimeria spp. (for example, Coccivac, Immucox, Paracox, Advent, and Nobilis Cox ATM), Eimeria spp. (for example, Inovocox), E. tenella (for example, Livacox), Toxoplasma gondii (for example, Ovilis and Toxovax), Pseudorabies virus (for example, Suvaxyn Aujeszky), Classical swine fever virus (for example, Porcilis Pesti and Bayovac CSF E2), Equine influenza virus (for example, PROTEQ-FLU and Recombitek), Newcastle disease virus (for example, Vectormune FP-ND), Avian influenza virus (for example, Poulvac FluFend I AI H5N3 RG), Avian influenza virus (for example, Trovac AI H5), Rabies virus (for example, Raboral and Purevax Feline Rabies), Feline leukemia virus (for example, EURIFEL FeLV), Canine parvovirus 1 (for example, RECOMBITEK Canine Parvo), Canine coronavirus (for example, RECOMBITEK Corona MLV), Canine distemper virus (for example, RECOMBITEK rDistemper and PUREVAXFerret Distemper), 1HN virus (for example, Apex-IHN). Other examples of veterinarian vaccines include reproduction control vaccines such as LHRH (for example, Vaxstrate, Improvac, Equito, Canine gonadotropinreleasing factor immunotherapeutic, and GonaCon) and Androstenedione (for example, Fecundin, Androvax and Ovastim).

In one embodiment of the invention, the antigens included in the multivalent vaccine composition of the invention are in the form of a pre-formulated influenza vaccine. In general, commercial influenza vaccines comprise inactivated whole virions or split virions. In one embodiment, therefore, the invention provides for a multivalent vaccine comprising a PapMV component, optionally a porin component, and an inactivated whole virion or split virion influenza vaccine. In a specific embodiment, the invention provides for a multivalent vaccine comprising PapMV VLPs and an inactivated whole split virion influenza vaccine.

Commercially available influenza vaccines are also typically trivalent in that they provide protection against three strains of influenza—in general strains of influenza A and influenza B. For example, for the 2007-2008 season, the strains were A/Solomon Islands/3/2006 (H1N1)-like, A/Wisconsin/67/2005 (H3N2)-like, and B/Malaysia/2506/2004-like; and for the 2008-2009 season the strains were A/Brisbane/59/2007 (H1N1); A/Brisbane/10/2007 (H3N2) and B/Florida/4/2006.

Influenza vaccines that are presently commercially available include, but are not limited to, Fluzone® and Vaxigrip® (Sanofi-aventis), Fluvirin® (Novartis Vaccine), Fluarix®, FluLaval® and Fluviral S/F® (GlaxoSmithKline), Afluria (CSL Biotherapies), FluMist® (MedImmune), and Influvac™ (Solvay Pharma).

The one or more antigens for inclusion in the multivalent vaccine compositions of the invention can also be in the form of another PapMV-based immunogenic preparation, for example, as a PapMV-antigen fusion. In these PapMV-antigen fusions, the antigen is attached to the coat protein of the PapMV, for example by genetically fusing the sequence encoding the antigen to the sequence encoding the coat protein in a position such that the antigen is exposed on the surface of the VLP once the recombinant coat protein self-assembles. For example, the antigen may be fused to the N-terminus or C-terminus of the coat protein or inserted into an internal loop. The recombinant fusion protein thus self-assembles into a VLP that presents the antigen on its surface. Examples of such fusions are provided in International Patent Application Nos. PCT/CA03/00985 (published as WO 2005/004761); PCT/CA2007/002069 (published as WO 2008/058396) and PCT/CA2007/001904 (published as WO 2008/058369), and in U.S. patent application Ser. No. 11/556,678 (published as US 2007/0166322) (each herein expressly incorporated by reference in their entirety).

### Optional Porin Component

As described above, the multivalent vaccine compositions can optionally include a porin component, which can be an OmpC, an OmpF, or a combination thereof, in addition to the PapMV component and one or more antigens.

In one embodiment of the present invention, the multivalent vaccine composition comprises a Salmonella spp. OmpC as a porin component. The porin component can be combined with the PapMV component to provide the PapMV-porin or it can be conjugated to the PapMV component to provide the PapMV-porin, as described in more detail below. In one embodiment, the multivalent vaccine comprises a PapMV-porin that comprises a Salmonella spp. OmpC conjugated to PapMV VLPs.

In one embodiment of the invention, the multivalent vaccine composition comprises PapMV-porin and one or more antigens from a pathogen, infection with which requires the participation of antibody and T cell immune responses in order to be effectively overcome. Examples of such pathogens include, but are not limited to, influenza virus, human papilloma virus (HPV), hepatitis B virus (HBV), hepatitis C virus (HCV), human immunodeficiency virus (HIV), human T-lymphotropic virus (HTLV), Dengue virus, Plasmodium falciparium (the causative agent of malaria), and bacteria that cause systemic infections (such as those that occur in typhoid fever, Leishmania major infection or Mycobacterium tuberculosis infection).

In another embodiment of the invention, the multivalent vaccine composition comprises PapMV-porin and one or more antigens from the influenza virus. In a specific embodiment of the invention, the multivalent vaccine composition comprises PapMV-porin and one or more antigens from the influenza virus and is capable of inducing an immune response that provides protection against multiple influenza strains, as well as against S. typhi. In a specific embodiment of the invention, the multivalent vaccine composition comprises PapMV-porin and an influenza vaccine and is capable of inducing an immune response that provides protection against multiple influenza strains, including strains against which the vaccine alone does not provide protection.

### OmpC

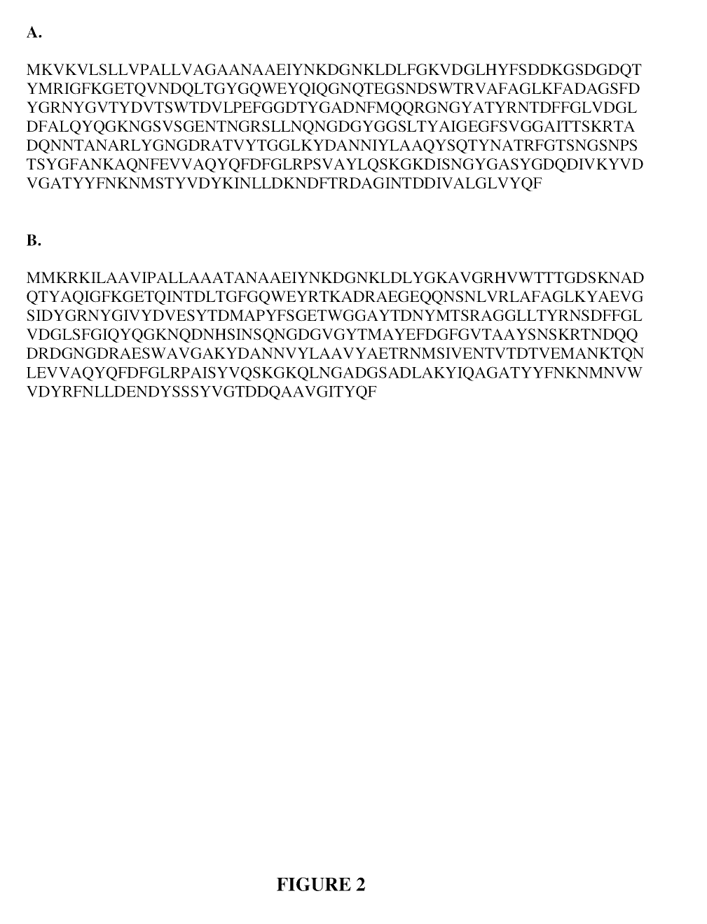

The sequences of OmpC porins from various S. typhi strains are known in the art and are readily accessible, for example, from GenBank database maintained by the National Center for Biotechnology Information (NCBI). For example, GenBank Accession No. P0A264 (SEQ ID NO:4; also shown in FIG. 2), GenBank Accession No. AA068302.1 and GenBank Accession No. NP_{—}804453: OmpC (S. enterica subsp. enterica serovar Typhi Ty2); and GenBank Accession No. CAD07499.1 and GenBank Accession No. NP_{—}456812.1: OmpC (S. enterica subsp. enterica serovar Typhi strain CT18).

The OmpC porin for use in the multivalent vaccine compositions can be obtained from Salmonella typhi by standard purification methods, or it can be a recombinant version of OmpC that is produced in heterologous cells or in vitro. The coding sequence for S. typhi OmpC is also known in the art (see GenBank Accession No. AL627274.1, in which the complement of nucleotides 21394-22530 represents the coding sequence for OmpC from S. enterica subsp. enterica serovar Typhi strain CT18; and GenBank Accession No. AE014613.1, in which nucleotides 681183-682319 represent the coding sequence for OmpC from S. enterica subsp. enterica serovar Typhi Ty2).

The OmpC porin incorporated into the multivalent vaccine composition can be the full-length protein or it can be a substantially full-length protein (for example, a protein comprising a N-terminal and/or C-terminal deletion of about 25 amino acids or less, about 20 amino acids or less, about 15 amino acids or less, or about 10 amino acids or less) that retains the adjuvant activity of the wild-type porin. The full-length protein can be the precursor form of OmpC (for example, as shown in FIG. 2 [SEQ ID NO:4]) or the mature (processed) form of OmpC in which the N-terminal leader (or signal) sequence has been removed (for example, the sequence represented by amino acids 22-378 of SEQ ID NO:4).

One skilled in the art will appreciate that the sequence of the OmpC porin may be varied slightly from the wild-type sequence (i.e. it may be a modified or “variant sequence”) without affecting the ability of the protein to function in the multivalent vaccine composition. For example, the OmpC porin may comprise one or more mutations, such as, amino acid insertions, deletions or substitutions, provided that the porin retains its ability to act as an adjuvant. As is known in the art, native OmpC is a “beta-barrel” structure with long external loops and shorter internal (periplasmic) turns. In accordance with one embodiment of the invention in which the OmpC comprises a variant sequence, the OmpC variant retains a beta-barrel conformation.

When the OmpC comprises a variant sequence that contains an insertion or deletion, the insertion or deletion in general comprises 20 amino acids or less. In one embodiment of the invention, when the OmpC comprises a variant sequence that contains an insertion or deletion, the insertion or deletion comprises 15 amino acids or less. In another embodiment, when the OmpC comprises a variant sequence that contains an insertion or deletion, the insertion or deletion comprises 10 amino acids or less.

When the OmpC comprises a variant sequence that contains one or more amino acid substitutions, these can be “conservative” substitutions or “non-conservative” substitutions as described above in relation to the PapMV coat protein.

As is known in the art, insertions and deletions in the external loop regions of OmpC are well-tolerated (see, for example, Vega et al., Immunology (2003) 110:206-16). In one embodiment of the invention, the amino acid sequence of the OmpC porin incorporated in the multivalent vaccine composition is a variant sequence comprising an insertion, deletion or substitution in an external loop. In one embodiment of the invention, the amino acid sequence of the OmpC porin incorporated in the multivalent vaccine composition is a variant sequence comprising an insertion or deletion in an external loop in which the insertion or deletion comprises 20 amino acids or less. In another embodiment of the invention, the amino acid sequence of the OmpC porin incorporated in the multivalent vaccine composition is a variant sequence comprising one or more conservative substitutions. In another embodiment of the invention, the amino acid sequence of the OmpC porin incorporated in the multivalent vaccine composition is a variant sequence comprising one or more conservative substitutions in a beta-strand region.

In one embodiment of the invention, the OmpC porin incorporated in the multivalent vaccine composition is a full-length or substantially full-length OmpC that has an amino acid sequence that has 95% or greater sequence identity with the sequence of the S. typhi OmpC porin as shown in FIG. 2 [SEQ ID NO:4]. In other embodiments, the OmpC porin incorporated in the multivalent vaccine composition is a full-length or substantially full-length OmpC that has an amino acid sequence that has 96% or greater, 97% or greater, 98% or greater, or 99% or greater sequence identity with the sequence of the S. typhi OmpC porin as shown in FIG. 2 [SEQ ID NO:4].

As shown in Table 2, while the sequences of the S. typhi OmpC porin and OmpC orthologues from other enterobacteria are fairly highly conserved, the sequences Salmonella spp. are very highly conserved. In general, the amino acid sequences of OmpC porins from other species of Salmonella show at least 95% sequence identity with OmpC from S. typhi. Accordingly, one embodiment of the invention provides for a porin component of the multivalent vaccine composition that comprises an OmpC porin from a Salmonella species other than S. typhi. Additional examples to those provided in Table 2 include, but are not limited to, OmpC from S. enterica serovar Typhimurium (GenBank Accession No. 16761195); OmpC from S. enterica serovar Typhi (GenBank Accession No. 47797); OmpC from S. enterica serovar Minnesota (GenBank Accession No. 8953564); OmpC from S. enterica serovar Dublin (GenBank Accession No. 19743624) and OmpC from S. enterica serovar Gallinarum (GenBank Accession No. 19743622).

TABLE 2
Sequence Identity of OmpC and OmpC Orthologues
from Various Enterobacteria

| | | | % |
|---|---|---|---|
| Organism | Protein | Reference | Identity¹ |
| Salmonella typhimurium | OmpC | P0A263 | 100 |
| LT2 | | | |
| Salmonella bongori | ORF_2828 | coliBase²: | 99 |
| | (Putative OmpC) | GL026809³ | |
| Salmonella enteritidis | ORF_1402 | coliBase²: | 98 |
| PT4 | (Putative OmpC) | GL063386³ | |
| Salmonella gallinarum | ORF_222 | coliBase²: | 98 |
| 287/91 | (Putative OmpC) | GL064166³ | |
| Escherichia coli | OmpC | Q8XE41 | 80 |
| O157:H7 EDL933 | | | |
| Shigella dysenteriae | ORF_14 | coliBase²: | 78 |
| M131649 | (Putative OmpC) | GL018139³ | |
| (M131)] | | | |
| Shigella flexneri 2a | Omp1b | Q83QU7 | 78 |
| 2457T | | | |

¹% identity is relative to the S. typhi OmpC protein (GenBank Accession No. P0A264) and was determined using the BLASTP 2.2.3 [Apr. 24, 2002] program (Altschul, S. F., et al., (1997), Nucleic Acids Res. 25: 3389-3402).
²Nucleic Acids Research, 2004, Vol. 32, Database issue D296-D299.
³GL numbers as of Jan. 23, 2007.

### OmpF Porin

The sequences of OmpF porins from various S. typhi strains are also known in the art and are readily accessible, for example, from the GenBank database maintained by the NCBI. For example, GenBank Accession No. CAD05399 (SEQ ID NO:5; also shown in FIG. 2) and GenBank Accession No. NP_{—}455485.1: OmpF precursor protein (S. enterica subsp. enterica serovar Typhi CT18); GenBank Accession No. AA069550.1, GenBank Accession No. NP_{—}805701.1 and GenBank Accession No. Q56113.2: OmpF precursor protein (S. enterica subsp. enterica serovar Typhi Ty2); GenBank Accession No. CAA61905.1: OmpF protein (S. typhi); and GenBank Accession No. AAG09474: outer membrane protein F precursor (S. typhi).

The OmpF porin for incorporation into the multivalent vaccine composition according to the invention can be obtained from S. typhi by standard purification methods, or it can be a recombinant version of OmpF that is produced in heterologous cells or in vitro. The coding sequence for S. typhi OmpF is also known in the art (see GenBank Accession No. AL627268.1, in which the complement of nucleotides 241298-242389 represents the coding sequence for OmpF from S. enterica subsp. enterica serovar Typhi strain CT18; and GenBank Accession No. AE014613.1, in which nucleotides 1979688-1980779 represent the coding sequence for OmpF from S. enterica subsp. enterica serovar Typhi Ty2).

The OmpF porin incorporated into the product can be the full-length protein or it can be a substantially full-length protein (for example, a protein comprising a N-terminal and/or C-terminal deletion of about 25 amino acids or less, about 20 amino acids or less, about 15 amino acids or less, or about 10 amino acids or less) that retains the adjuvant activity of the wild-type porin. The full-length protein can be the precursor form of OmpF (for example, as shown in FIG. 2 [SEQ ID NO: 5]) or the mature (processed) form of OmpF in which the leader (or signal sequence has been removed (for example, the sequence represented by amino acids 23-363 of SEQ ID NO:5).

One skilled in the art will appreciate that the sequence of the OmpF porin incorporated in the multivalent vaccine composition may also be varied slightly from the wild-type sequence (i.e. it may be a modified or “variant sequence”) without affecting the ability of the protein to function in the multivalent vaccine composition. For example, the OmpF porin may comprise one or more mutations, such as, amino acid insertions, deletions or substitutions, provided that the porin retains its ability to act as an adjuvant. As is known in the art, native OmpF is a “beta-barrel” structure with long external loops and shorter internal (periplasmic) turns. In one embodiment, when the OmpF comprises a variant sequence, it also retains a beta-barrel conformation.

When the OmpF comprises a variant sequence that contains an insertion or deletion, the insertion or deletion in general comprises 20 amino acids or less, for example 15 amino acids or less, or 10 amino acids or less.

When the OmpF comprises a variant sequence that contains one or more amino acid substitutions, these can be “conservative” substitutions or “non-conservative” substitutions, as described above for OmpC. In one embodiment of the invention, the amino acid sequence of the OmpF porin incorporated in the multivalent vaccine composition is a variant sequence comprising an insertion, deletion or substitution in an external loop. In one embodiment of the invention, the amino acid sequence of the OmpF porin incorporated in the multivalent vaccine composition is a variant sequence comprising an insertion or deletion in an external loop in which the insertion or deletion comprises 20 amino acids or less. In another embodiment of the invention, the amino acid sequence of the OmpF porin incorporated in the multivalent vaccine composition is a variant sequence comprising one or more conservative substitutions.

In another embodiment of the invention, the amino acid sequence of the OmpF porin incorporated in the multivalent vaccine composition is a variant sequence comprising one or more conservative substitutions in a beta-strand region.

In another embodiment of the invention, the OmpF porin incorporated in the multivalent vaccine composition is a full-length or substantially full-length OmpF that has an amino acid sequence that has 95% or greater sequence identity with the sequence of the S. typhi OmpF porin as shown in FIG. 2 [SEQ ID NO:5]. In other embodiments, the OmpF porin incorporated in the multivalent vaccine composition is a full-length or substantially full-length OmpF that has an amino acid sequence that has 96% or greater, 97% or greater, 98% or greater sequence identity, or 99% or greater sequence identity with the sequence of the S. typhi OmpF porin as shown in FIG. 2 [SEQ ID NO:5].

As shown in Table 3, while the sequences of the S. typhi OmpF porin and OmpF orthologues from other enterobacteria are fairly highly conserved, the sequences Salmonella spp. are very highly conserved. In general, the amino acid sequences of OmpF porins from other species of Salmonella show at least 95% sequence identity with OmpF from S. typhi. Accordingly, one embodiment of the invention provides for a porin component of the multivalent vaccine composition that comprises an OmpC porin from a Salmonella species other than S. typhi.

TABLE 3
Sequence Identity of OmpF and OmpF Orthologues
from Various Enterobacteria

| | | | % |
|---|---|---|---|
| Organism | Protein | Reference | Identity¹ |
| Salmonella enteritidis PT4 | ORF_34 | coliBase²: | 100 |
| | | GL060731³ | |
| Salmonella gallinarum 287/91 | ORF_21 | coliBase²: | 99 |
| | | GL069216³ | |
| Salmonella typhimurium DT104 | ORF_287 | coliBase²: | 99 |
| | | GL0044362³ | |
| Salmonella bongori | ORF_1160 | coliBase²: | 98 |
| | | GL025398³ | |
| Escherichia coli DH10B | ORF_2 | coliBase²: | 58 |
| | | GL037694³ | |
| Shigella flexneri 2a 2457T | OmpF | Q83RY7 | 58 |

¹% identity is relative to the S. typhi OmpF protein (GenBank Accession No. CAD05399) and was determined using the BLASTP 2.2.3 [Apr. 24, 2002] program (Altschul, S. F., et al, (1997), Nucleic Acids Res. 25: 3389-3402.
²Nucleic Acids Research, 2004, Vol. 32, Database issue D296-D299.
³GL numbers as of Jan. 23, 2007.

### Preparation of Ompc and Ompf

The OmpC and/or OmpF porins can be purified from S. typhi using standard techniques known in the art. An example of such a technique has been described by Salazar-Gonzalez et al. in Immunol. Lett. (2004) 93:115-122 (herein expressly incorporated by reference in its entirety). A representative method is also provided herein as Example 1. In order to obtain a preparation of OmpC that is substantially free of OmpF, an OmpF knockout mutant strain of S. typhi may be used. For example, Salmonella strain STYF302 (ΔompF KmR) (Martinez-Flores et al., J. Bacteriol. (1999) 181:556-562). Similarly, in order to obtain a preparation of OmpF that is substantially free of OmpC, an OmpC knockout mutant strain of S. typhi may be used. For example, Salmonella strain STYC171 (ΔompC KmR) (Martinez-Flores et al., ibid.).

In general, porin purification from S. typhi involves first growing the bacteria in a suitable medium under suitable conditions until an acceptable density has been achieved, for example, to an OD₅₄₀ of between about 0.8 and about 1.5. The cells are harvested and lysed and the OmpC and/or OmpF porin extracted by a series of centrifugation and homogenisation steps. The porin(s) can be further purified by standard chromatography, for example, fast protein liquid chromatography (FPLC) or medium-pressure liquid chromatography (MPLC), using size-exclusion, gel filtration or other medium. Both OmpC and OmpF preparations are generally stable and can be stored at 4° C. for extended periods of time, for example, for periods of 4 weeks or more. In one embodiment of the invention in which OmpC and OmpF were prepared essentially as described in Example 1, the porin preparation was stable at 4° C. for one year or more.

The porins can also be prepared by standard genetic engineering techniques by the skilled worker provided with the sequence of the wild-type protein(s). Methods of cloning and expressing recombinant proteins are well known in the art (see, for example, Ausubel et al. (1994 & updates) Current Protocols in Molecular Biology, John Wiley & Sons, New York), as are the sequences of the wild-type OmpC and OmpF proteins (see SEQ ID NOs:4 and 5).

Isolation and cloning of the nucleic acid sequence encoding the OmpC or OmpF wild-type protein can be achieved using standard techniques (see, for example, Ausubel et al., ibid.). For example, the nucleic acid sequence can be obtained directly from S. typhi by standard techniques (for example, by PCR-based techniques). The nucleic acid sequence encoding the relevant porin protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The porin can then be expressed and purified using standard techniques.

When desired, the nucleic acid sequence encoding the OmpC or OmpF porin protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

One of ordinary skill in the art will appreciate that the DNA encoding the porin protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as those described above with respect to the cloning and expression of the PapMV coat protein. As also described above, the expression vector may additionally contain heterologous nucleic acid sequences that facilitate the purification of the expressed protein.

The expression vector can be introduced into a suitable host cell by one of a variety of methods known in the art and described above. The recombinant porin protein can be isolated from the host cells by standard methods such as those described above for the wild-type proteins or following other published protocols (see, for example, Arockiasamy, et al. Anal. Biochem. (2000) 283:64-70; Vega, et al. Immunology (2003) 110:206-216). The protein can be further purified by standard techniques, such as chromatography, to remove contaminating host cell proteins or other compounds, such as LPS. In one embodiment of the present invention, the porin protein is purified to remove LPS.

### Optional Conjugation of the PapMV and Porin Components

As noted above, when the multivalent vaccine composition includes a porin component, this porin component can optionally be conjugated to the PapMV component via the coat protein of PapMV or PapMV VLP. Conjugation can be, for example, binding via covalent, non-covalent or affinity means.

In one embodiment, the porin component is conjugated to the PapMV component by affinity means. In accordance with this embodiment, the PapMV VLP comprises an affinity moiety, such as a peptide, that is exposed on the surface of the VLP following self-assembly, and which is capable of specifically binding to the porin component. The affinity moiety may be genetically fused (in the case of a peptide or protein fragment), or covalently or non-covalently attached to the PapMV or VLP. Binding of the porin component to the affinity moiety should not interfere with the recognition of the porin by the host's immune system.

In one embodiment of the invention, the porin component is conjugated to the PapMV component via an affinity peptide that has been genetically fused to the PapMV coat protein. In accordance with this embodiment and in order to allow presentation of the peptide on the surface of the assembled VLP for conjugation with the porin component, the peptide is preferably attached to a region of the coat protein that is disposed on the outer surface of the VLP. Thus the peptide can be genetically fused proximal to, or at, the amino-(N-) or carboxy-(C-) terminus of the coat protein, or it can be inserted into, or attached to, an internal loop of the coat protein which is disposed on the outer surface of the VLP. An example of such a loop would be the region comprised by amino acids 49 to 52 of the PapMV coat protein as set forth on SEQ ID NO:1. In one embodiment of the present invention, the peptide is genetically fused at the C-terminus of the PapMV coat protein.

Examples of suitable affinity moieties include, but are not limited to, antibodies and antibody fragments (such as Fab fragments, Fab′ fragments, Fab′-SH, fragments F(ab′)2 fragments, Fv fragments, diabodies, and single-chain Fv (scFv) molecules), streptavidin (to bind a proin component labelled with biotin), affinity peptides or affinity protein fragments that specifically bind the porin component.

Suitable peptides or antibodies (including antibody fragments) for use as affinity moieties can be selected by art-known techniques, such as phage or yeast display techniques. The peptides can be naturally occurring, recombinant, synthetic, or a combination of these. For example, the peptide can be a fragment of a naturally occurring protein or polypeptide. The term peptide also encompasses peptide analogues, peptide derivatives and peptidomimetic compounds. Such compounds are well known in the art and may have advantages over naturally occurring peptides, including, for example, greater chemical stability, increased resistance to proteolytic degradation, enhanced pharmacological properties (such as, half-life, absorption, potency and efficacy) and/or reduced antigenicity.

Suitable peptides can range from about 3 amino acids in length to about 50 amino acids in length. In accordance with one embodiment of the invention, the PapMV component comprises an affinity peptide that is at least 5 amino acids in length. In accordance with another embodiment of the invention, the PapMV component comprises an affinity peptide that is at least 7 amino acids in length. In accordance with another embodiment of the invention, the PapMV component comprises an affinity peptide that is between about 5 and about 50 amino acids in length. In accordance with another embodiment of the invention, the PapMV component comprises an affinity peptide that is between about 7 and about 50 amino acids in length. In other embodiments of the present invention, the PapMV component comprises an affinity peptide that is between about 5 and about 45 amino acids in length, between about 5 and about 40 amino acids in length, between about 5 and about 35 amino acids in length and between about 5 and about 30 amino acids in length. In accordance with a specific embodiment of the invention, the PapMV component comprises an affinity peptide that is 3, 4, 5, 6, 7, 8, 9, 10, 11, 12, 13, 14 or 15 amino acids in length. As would be understood by a worker skilled in the art, the length of the peptide selected for binding the porin component should not interfere with the ability of the PapMV VLP to self-assemble or with the recognition of the porin, once bound, by the host's immune system.

Affinity moieties comprised by the PapMV or VLP can be single peptides or can be a tandem or multiple arrangement of peptides. A spacer can be included between the affinity moiety and the coat protein if desired in order to facilitate the binding of large antigens. Suitable spacers include short stretches of neutral amino acids, such as glycine. For example, a stretch of between about 3 and about 10 neutral amino acids.

Phage display can be used to select specific peptides that bind to the porin of interest using standard techniques (see, for example, Current Protocols in Immunology, ed. Coligan et al., J. Wiley & Sons, New York, N.Y.) and/or commercially available phage display kits (for example, the Ph.D. series of kits available from New England Biolabs, and the T7-Select® kit available from Novagen). An example of selection of peptides by phage display is also provided in Example 5, below.

Representative peptides that bind OmpC or OmpF porins identified by phage display are shown in Table 4. One skilled in the art will appreciate that these peptides are examples only and that other peptides having an affinity for a porin of interest can be readily identified using art-known techniques such as those described above. Truncated versions, for example comprising at least 4 consecutive amino acids, of the sequences set forth in Table 4 that retain the ability to bind a porin protein are also contemplated. In accordance with a specific embodiment of the present invention, the PapMV component of the PapMV-porin includes one or more affinity peptides comprising all or a part of the sequence set forth in SEQ ID NO:10, SEQ ID NO:11, SEQ ID NO:12, SEQ ID NO:13, SEQ ID NO:14 or SEQ ID NO:15.

TABLE 4
Affinity Peptides Selected by Phage Display and their Frequency.

| Target | Selected affinity peptides | SEQ ID NO |
|---|---|---|
| OmpC | SLSLIQT | 10 |
| OmpC | EAKGLIR | 11 |
| OmpC | TATYLLD | 12 |
| OmpF | FHENWPS | 13 |
| OmpF | FHEFWPT | 14 |
| OmpF | FHEXWPT, where X is N or F | 15 |

In another embodiment, the porin component can be chemically cross-linked to the coat protein, for example, by covalent or non-covalent (such as, ionic, hydrophobic, hydrogen bonding, or the like) attachment. The porin and/or coat protein can be modified to facilitate such cross-linking as is known in the art, for example, by addition of a functional group or chemical moiety to the protein and/or porin, for example at the C- or N-terminus or at an internal position. Exemplary modifications include the addition of functional groups such as S-acetylmercaptosuccinic anhydride (SAMSA) or S-acetyl thioacetate (SATA), or addition of one or more cysteine residues. Other cross-linking reagents are known in the art and many are commercially available (see, for example, catalogues from Pierce Chemical Co. and Sigma-Aldrich). Examples include, but are not limited to, diamines, such as 1,6-diaminohexane, 1,3-diamino propane and 1,3-diamino ethane; dialdehydes, such as glutaraldehyde; succinimide esters, such as ethylene glycol-bis(succinic acid N-hydroxysuccinimide ester), disuccinimidyl glutarate, disuccinimidyl suberate, N-(g-Maleimidobutyryloxy) sulfosuccinimide ester and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; 3-maleimidopropionic acid N-hydroxysuccinimide ester, and the like. Many of the above-noted cross-linking agents incorporate a spacer that distances the porin from the VLP. The use of other spacers is also contemplated by the invention. Various spacers are known in the art and include, but are not limited to, 6-aminohexanoic acid; 1,3-diamino propane; 1,3-diamino ethane; and short amino acid sequences, such as polyglycine sequences, of 1 to 5 amino acids.

To facilitate covalent attachment of the porin component to the coat protein of the VLP, the coat protein can be genetically fused to a short peptide or amino acid linker that is exposed in the surface of the VLP in a similar manner to the affinity peptides described above and that provides an appropriate site for chemical attachment of the porin. For example, short peptides comprising cysteine residues, or other amino acid residues having side chains that are capable of forming covalent bonds (for example, acidic and basic residues) or that can be readily modified to form covalent bonds as known in the art. The amino acid linker or peptide can be, for example, between one and about 20 amino acids in length. In one embodiment, the coat protein is fused with a short peptide comprising one or more lysine residues, which can be covalently coupled, for example with a cysteine residue in the porin through the use of a suitable cross-linking agent as described above. In another embodiment, the coat protein is fused with a short peptide sequence of glycine and lysine residues, for example, a peptide comprising the sequence: GGKGG.

### Evaluation of Efficacy

The ability of the multivalent vaccine compositions of the present invention to induce an immune response in an animal can be tested by art-known methods, such as those described below and in the Examples. For example, the multivalent vaccine composition can be administered to a suitable animal model, for example by subcutaneous injection or intranasally, and the development of specific antibodies to the porin component and the one or more antigens evaluated by standard techniques, such as Enzyme-Linked Immunosorbent Assay (ELISA).

Cellular immune responses can also be assessed by techniques known in the art. For example, the cellular immune response can be determined by evaluating processing and cross-presentation of an epitope comprised by the vaccine to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines (see, for example, Leclerc, D., et al., J. Virol, 2007, 81(3):1319-26).

Challenge studies can also be conducted to assess the protection provided by the multivalent vaccine composition against the relevant disease causing agents. Such studies involve the inoculation of groups of test animals (such as mice, ferrets or non-human primates) with a multivalent vaccine composition of the invention by standard techniques. Control groups comprising non-inoculated animals and/or animals inoculated with, for example, antigen(s) alone, a commercially available vaccine or a positive control, are set up in parallel. After an appropriate period of time post-vaccination, the animals are challenged with one of the relevant disease causing organisms. Blood samples collected from the animals pre- and post-inoculation, as well as post-challenge are then analyzed for an antibody response to the organism. Suitable tests for the antibody response include, but are not limited to, Western blot analysis and ELISA. The animals can also be monitored for development of the disease associated with the organism. Challenge studies that test the ability of the vaccine to protect against the other strain(s) or organism(s) of interest can be conducted in separate groups of test animals either subsequently or in parallel.

### Vaccine Compositions

The multivalent vaccine compositions of the invention are generally formulated with a suitable carrier, excipient or the like, for administration to the subject to be treated. The PapMV component, the one or more antigens and the optional porin component may be formulated separately or they may be formulated together. The vaccine compositions may optionally comprise one or more other standard components of pharmaceutical compositions that improve the stability, palatability, pharmacokinetics, bioavailability or the like, of the product. The invention also provides for formulations of the PapMV component or the PapMV-porin with a suitable carrier, excipient or the like, for use as an adjuvant.

The compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the composition to the mucous membranes of the nasal passage or nasal cavity of the subject. In one embodiment of the present invention, the compositions are formulated for oral or parenteral administration or for administration by inhalation or spray, for example by an intranasal route. In another embodiment, the compositions are formulated for parenteral administration. In a further embodiment, compositions are formulated for subcutaneous or intramuscular administration. A non-limiting example of a formulation of OmpC suitable for subcutaneous or intramuscular administration is provided by Salazar-Gonzales, et al., Immunol. Lett. (2004) 93:115-122).

The compositions also preferably comprise an effective amount of the PapMV component. The term “effective amount” as used herein refers to an amount of the PapMV component required to produce a detectable immune response when administered as part of the multivalent vaccine. In one embodiment of the present invention, a unit dose of the multivalent vaccine composition comprises between about 1 μg to about 10 mg of PapMV coat protein. In another embodiment, a unit dose of the multivalent vaccine composition comprises between about 10 μg to about 10 mg of coat protein. In a further embodiment, a unit dose of the multivalent vaccine composition comprises between about 10 μg to about 5 mg of coat protein. In another embodiment, a unit dose of the multivalent vaccine composition comprises between about 40 μg to about 2 mg of coat protein.

The compositions preferably comprise an effective amount of the porin component. In one embodiment of the present invention, in which the multivalent vaccine composition comprises OmpC, a unit dose comprises between about 1 μg to about 10 mg of OmpC protein. In another embodiment, in which the multivalent vaccine composition comprises OmpC, the unit dose comprises between about 1 μg to about 5 mg of OmpC protein. In other embodiments, in which the multivalent vaccine composition comprises OmpC, the unit dose comprises between about 1 μg to about 2 mg, between about 1 μg to about 1 mg, between about 1 μg to about 90 μg, between about 1 μg to about 80 μg, between about 1 μg to about 70 μg, between about 1 μg to about 60 μg or between about 1 μg to about 50 μg of OmpC protein.

The effective amount of each component of the multivalent vaccine composition, including the one or more antigens, as well as the effective amount of the final vaccine composition, for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. One or more doses may be used to immunize the animal, and these may be administered on the same day or over the course of several days or weeks.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the immunogenic composition in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the immunogenic composition is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the porin preparation and optional antigenic material in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the porin preparation and optional antigenic material in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the immunogenic composition in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

The compositions can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the PapMV component, optional porin component and/or one or more antigens with one or more sweetening agents, for example glycerol, propylene glycol, sorbitol or sucrose. Such formulations can also optionally contain one or more demulcents, preservatives, flavouring agents and/or colouring agents.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the multivalent vaccine composition in accordance with the present invention may contain preservatives such as antimicrobial agents, anti oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum albumin or skimmed milk) together with a suitable buffer (e.g. phosphate buffer). The pH and exact concentration of the various components of the composition may be adjusted according to well-known parameters.

Other pharmaceutical compositions and methods of preparing pharmaceutical compositions are known in the art and are described, for example, in “Remington: The Science and Practice of Pharmacy” (formerly “Remingtons Pharmaceutical Sciences”); German), A., Lippincott, Williams & Wilkins, Philadelphia, Pa. (2000).

### Uses

The present invention provides for the use of, and methods of using, the multivalent vaccine compositions to provide protection against a plurality of strains of a given pathogen, or against more than one pathogen.

In one embodiment, the present invention provides for the use of, and methods of using, the multivalent vaccine compositions to provide protection against a plurality of influenza virus strains. In a specific embodiment, the one or more antigens included in the multivalent vaccine composition are in the form of a pre-formulated influenza vaccine and the PapMV component and optional porin component act to adjuvant the effects of the pre-formulated vaccine such that it provides protection against heterologous strains of influenza. In another embodiment, the present invention provides for the use of PapMV VLPs to adjuvant a pre-formulated influenza vaccine such that it provides protection against heterologous strains of influenza.

When the multivalent vaccine comprises a PapMV component and a pre-formulated influenza vaccine, the ratio of the PapMV component to influenza vaccine can be between about 1:1 and about 1:100 (by weight). As shown in the Examples provided herein, as the amount of PapMV VLPs (as the PapMV component) is increased in relation to the influenza vaccine, the adjuvant effect of PapMV VLPs demonstrates saturation at a vaccine:PapMV VLP ratio of about 1:10 (by weight). As such, the amount of PapMV VLPs can be kept relatively low while still providing strong adjuvant effects. Accordingly, in one embodiment, the ratio of the influenza vaccine to PapMV VLPs can be between about 1:1 and about 1:20 by weight. In other embodiments, the ratio of the influenza vaccine to PapMV VLPs is between about 1:2 and about 1:20 by weight, for example, between about 1:2 and about 1:15 by weight; between about 1:3 and about 1:15 by weight; between about 1:4 and about 1:15 by weight, or between about 1:5 to about 1:15 by weight. In another embodiment, the ratio of the influenza vaccine to PapMV VLPs is between about 1:1 and about 1:10 by weight.

In one embodiment of the invention, the multivalent vaccine compositions comprise antigens from more than one disease causing agent and thus provide protection against more than one disease or disorder. Examples of combinations of antigens, which include pre-formulated vaccines comprising the combinations, include, but are not limited to, antigens from hepatitis A and hepatitis B viruses to provide a multivalent vaccine against hepatitis A/hepatitis B; antigens from hepatitis B virus and H. influenzae type b to provide a multivalent vaccine against hepatitis B/Hib; antigens from Corynebacterium diphtheriae and Clostridium tetani to provide a multivalent vaccine against tetanus and dipheria; antigens from Corynebacterium diphtheriae, Clostridium tetani and Bordetella pertussis to provide a multivalent vaccine against diphtheria/tetanus/pertussis; antigens from Corynebacterium diphtheriae, Clostridium tetani, Bordetella pertussis and H. influenzae type b to provide a multivalent vaccine against diphtheria/tetanus/pertussis/Hib; antigens from Corynebacterium diphtheriae, Clostridium tetani, Bordetella pertussis, polio virus and hepatitis B virus to provide a multivalent vaccine against diphtheria/tetanus/pertussis/polio/hepatitis B; antigens from the mumps virus, measles virus and rubella virus to provide a multivalent vaccine against measles/mumps/rubella (MMR), and antigens from the mumps virus, measles virus, rubella virus and varicella zoster virus to provide a multivalent vaccine against MMR/chickenpox.

In one embodiment, the present invention provides for the use of, and methods of using, the multivalent vaccine compositions to provide protection against S. typhi infection and protection against the one or more other disease-causing agents. In accordance with this embodiment, the multivalent vaccine compositions comprise a PapMV component, one or more antigens and a porin component. The invention also provides for the use of the PapMV-porin as an adjuvant to potentiate the immunogenic effect of one or more antigens.

In a specific embodiment of the invention, the multivalent vaccine compositions comprising a PapMV component, one or more antigens and a porin component can be used in the prevention or treatment of a variety of diseases or disorders in addition to providing protection against S. typhi infection, depending on the antigen(s) selected for inclusion in the multivalent vaccine composition. Non-limiting examples include various virally- or bacterially-related diseases, such as influenza (using antigenic material from various influenza viruses), HCV infections (using HCV antigenic material), HBV infections (using HBV antigenic material), HAV infections (using HAV antigenic material), HIV infections (using HIV antigenic material), polio (using poliovirus antigenic material), diptheria (using antigenic material derived from diptheria toxin), tuberculosis (using Mycobacterium tuberculosis antigenic material), EBV infections (using EBV antigenic material), as well as allergic reactions (using various allergens) and cancer (using various tumour-associated antigens). Other uses include, for example, prevention or treatment of inflammatory diseases (for example, arthritis); infections by avian flu virus, human respiratory syncytial virus, Dengue virus, measles virus, mumps virus, rubella virus, Varicella zoster virus, variola virus, herpes simplex virus, human papillomavirus, pseudorabies virus, swine rotavirus, swine parvovirus, Newcastle disease virus, foot and mouth disease virus, hog cholera virus, African swine fever virus, infectious bovine rhinotracheitis virus, infectious laryngotracheitis virus, La Crosse virus, neonatal calf diarrhea virus, bovine respiratory syncytial virus, bovine viral diarrhea virus, Mycoplasma hyopneumoniae, Streptococcal bacteria, Gonococcal bacteria, Enterobacteria or parasites (for example, leishmania or malaria).

As is known in the art, typhoid fever is most prevalent in third world and/or tropical countries, as are a number of other diseases, such as, amoebic dysentery (amoebiasis), shigellosis, cholera, meningococcal meningitis, yellow fever, Dengue fever, encephalitis, West Nile virus disease, hepatitis, malaria, rotavirus infections, human papilloma virus infections, Chlamydia infections, SARS infections, Vancomycin-resistant S. aureus infections, Cryptosporidiosis, Hanta virus infections, Epstein Barr virus infections, Cytomegalovirus infections, H5N1 Influenza, Enterovirus 71 infections, E. coli O157:H7 infections, Human Monkey pox, Lyme disease, Cyclosporiasis, Hendra virus infections, Nipah virus infections, Rift Valley fever, Plague, Marburg haemorrhagic fever, Whitewater arrollo virus infections and the like. One embodiment of the invention provides for multivalent vaccine compositions that include antigen(s) from one or more of the causative agents of the diseases listed above and for the use of these vaccines to provide protection against S. typhi infection and protection against one or more of amoebic dysentery, shigellosis, cholera, meningococcal meningitis, yellow fever, Dengue fever, encephalitis, West Nile virus disease, hepatitis, or malaria. Such multivalent vaccines are not only useful for individuals who live in countries where such diseases are prevalent, but also for travellers planning to visit countries where these diseases are prevalent.

In a specific embodiment, the invention provides for a multivalent vaccine composition comprising PapMV-porin and one or more antigens derived from the influenza virus and for the use of this vaccine to provide protection against typhoid fever and influenza. In another embodiment, the invention provides for a multivalent vaccine composition comprising PapMV-porin in combination with a commercial influenza vaccine, for use to provide protection against typhoid fever and influenza.

The multivalent vaccine compositions of the invention are suitable for use in humans as well as non-human animals, including domestic and farm animals. The administration regime for the vaccine may be similar to other generally accepted vaccination programs. For example, a single administration of the product in an amount sufficient to elicit an effective immune response may be used or, alternatively, other regimes of initial administration of the vaccine followed by boosting with antigen alone or with the vaccine, the PapMV component or the PapMV-porin may be used. When the multivalent vaccine composition is a combination product comprising a PapMV component or a PapMV-porin and a commercial vaccine, the PaMV component/PapMV-porin may be combined with the commercial vaccine and administered as a single composition, with the option of subsequent boosters with the PapMV component/PapMV-porin alone, the commercial vaccine alone or a combination of the two. Alternatively, the PapMV component/PapMV-porin may be administered separately from the commercial vaccine. In this case, the PapMV component/PapMV-porin may be administered prior to or subsequent to administration with the commercial vaccine. Optional boosters of either the PapMV component/PapMV-porin or the commercial vaccine or both may also be included in the regime. Boosting in either administration regime may occur at times that take place well after the initial administration, for example, if antibody titres fall below acceptable levels. The exact mode of administration of the product will depend for example on the components of the multivalent vaccine composition, the subject to be treated and the desired end effect of the treatment. Appropriate modes of administration can be readily determined by the skilled practitioner.

The multivalent vaccine composition can be used prophylactically, for example to prevent infection by a virus, bacteria or other infectious particle, or development of a tumour or other disease, or it may be used therapeutically to ameliorate the effects of a disease or disorder associated with an infection or of a cancer or other disease. In one embodiment of the invention, the multivalent vaccine composition is used prophylactically.

As demonstrated herein, the multivalent vaccines of the invention are capable of providing a long-lasting immune response that confers protection on the vaccinated subject for a period of several months after vaccination. In one embodiment of the invention, therefore, the multivalent vaccine composition is used prophylactically to provide a long-lasting immune response capable of protecting the vaccinated subject for a period of several months, for example, between about 2 months and about 10 months, after vaccination. In a specific embodiment in which the multivalent vaccine comprises one or more influenza virus antigens, the multivalent vaccine is used to provide protection in a subject against infection with an influenza virus for 6 months or more, for example, at least 7, at least 8, at least 9, or at least 10 months after vaccination.

### Kits

The present invention additionally provides for kits comprising a multivalent vaccine composition or a PapMV component or a PapMV-porin. Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the contents of the kit.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Purification of Salmonella typhi Porin Proteins

The following purification procedure was used for purification of the porins OmpC and OmpF from S. typhi. The purification procedure is based on that described by Secundino et al. (2006), Immunology 117:59.

The two proteins were co-purified from Salmonella typhi. Individual purification of OmpC and OmpF was achieved using knock-out mutants of S. typhi in which either OmpC [STYC171 (OmpC-)] or OmpF [STYF302 (OmpF-)] open reading frames have been interrupted. The procedure for purification of the individual porins from the knock-out mutated forms of the bacteria was followed as for the co-purification. This procedure is outlined below.

The bacterial strain, Salmonella typhi 9,12, Vi:d (ATCC 9993) was grown in Minimal medium A supplemented with yeast extract, magnesium and glucose at 37° C., 200 rpm. The formula for 10 L Minimal medium A supplemented with yeast extract, magnesium and glucose is: 5.0 g of dehydrated Na-Citrate (NaC₆H₅O₇:2H₂O), 31.0 g NaPO₄ monobasic (NaH₂PO₄), 70.0 g NaPO₄ dibasic (Na₂HPO₄), 10.0 g (NH₄)₂SO₄, 200 mL yeast extract solution 5% (15.0 g in 300 mL). 1.434 L medium was distributed per 4 L Erlenmeyer flask. Sterilization was performed at 12 PC, 15 lbs pressure/in², 15 min. To each flask was then added: 6.0 mL of a 25% (w/v) sterile MgSO₄ solution and 60.0 mL of a 12.5% (w/v) glucose solution. The flask was inoculated with an overnight culture of S. typhi and when the OD₅₄₀ reached 1.0, incubation was stopped and the culture centrifuged at 7,500 rpm for 15 min at 4° C. The pellet was resuspended in 100 mL final volume of Tris-HCl pH 7.7 (6.0 g Tris-base/L) and the biomass was sonicated for 90 min on ice and then centrifuged at 7,500 rpm for 20 min at 4° C. To each 10 mL of supernatant was added: 2.77 mL 1M MgCl₂, 25 ml RNaseA (10,000 U/mL) and 25 ml DNaseA (10,000 U/mL). The mixture was then incubated at 37° C., 120 rpm for 30 min.

Porin extraction from the mixture was performed as follows:

The mixture was ultracentrifuged at 45,000 rpm, 4° C. for 45 min, and the pellet retained. The pellet was resuspended in 10 mL 5 mL Tris-HCl containing 2% (w/v) SDS and then homogenised. The homogenised mixture was incubated at 32° C., 120 rpm for 30 min. The incubated mixture was ultracentrifuged at 40,000 rpm, 20° C. for 30 min, and the pellet retained. The pellet was resuspended in 5 mL Tris-HCl containing 2% (w/v) SDS and then homogenised. The homogenized pellet was incubated at 32° C., 120 rpm for 30 min. The incubated mixture was ultracentrifuged at 40,000 rpm, 20° C. for 30 min, and the pellet retained. The pellet was resuspended in 20 mL Nikaido buffer containing 1% (w/v) SDS and then homogenised. [For 1 L of Nikaido buffer containing 1% (w/v) SDS: 6.0 g Tris-base, 10.0 g SDS, 23.4 g NaCl, 1.9 g EDTA was dissolved in water and the pH adjusted to pH 7.7. 0.5 mL β-mercaptoethanol solution was then added]. The mixture was incubated at 37° C., 120 rpm for 120 min. The incubated mixture was ultracentrifuged at 40,000 rpm, 20° C. for 45 min. The supernatant, which contained the porin extract, was recovered.

The porins were purified from the supernatant using fast protein liquid chromatography (FPLC). 0.5× Nikaido buffer (see above) without β-mercaptoethanol was employed during the purification process. The proteins were separated using a Sephacryl S-200 (FPLC WATERS 650 E) with a Flux speed of 10 mL/min. The column was loaded with 22 mL of supernatant. Eluted fractions were monitored at 260 nm and 280 nm. The main peak, which contained the purified porins, was retained and stored at 4° C. The purified porins were stable for long period (over one year).

### Example 2

### Efficacy of a Vaccine Composition Comprising PapMV VLPs and OmpC

A candidate vaccine formulation composed of PapMV VLPs and OmpC combined in a 1:1 w/w ratio (10 μg of each), was demonstrated to provide mice with protection against 500LD₅₀ of S. typhi infection. OmpC was prepared as described in Example 1. PapMV VLPs were prepared according to standard purification procedure (Denis et al., 2007, Virology 363; 59-68). The PapMV coat protein used in the preparation of the VLPs harboured a deletion of the N-terminal 5 amino acids and had a 6×His tag at its C-terminus. A multiple cloning site had been introduced between the 6×His tag and the C-terminus of the coat protein that included SpeI and MluI restrictions sites, resulting in the addition of 5 amino acids (TSTTR) between the C-terminus and the His tag. The amino acid sequence of this coat protein (PapMV CPsm) is provided in FIG. 3(A) [SEQ ID NO:37] and the nucleotide sequence encoding the PapMV CPsm protein is provided in FIG. 3(B) [SEQ ID NO:38].

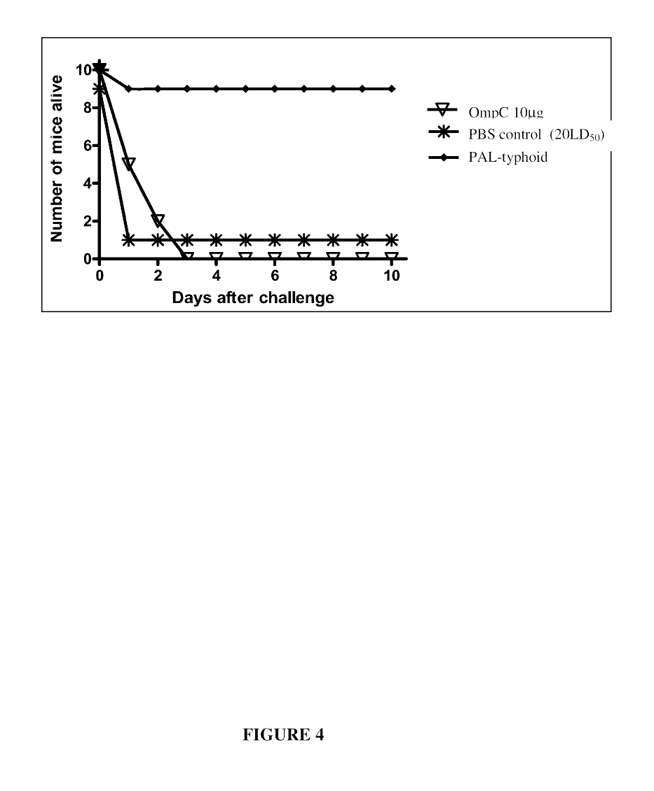

Mice, 10 per group, were vaccinated with a physiologic solution (PBS), with 10 μg of OmpC alone or with the PapMV VLP-OmpC vaccine. Control mice vaccinated with PBS were challenged with 20LD₅₀ of S. typhi and mice vaccinated with OmpC or the PapMV VLP-OmpC vaccine were challenged with 500LD₅₀ of S. typhi. All challenges were via the i.p. route. The results as shown in FIG. 4 demonstrate that the PapMV VLP-OmpC vaccine protects mice against S. typhi infection. This is a very promising result indicating that this candidate vaccine could be an effective option for controlling S. typhi infection.

### Example 3

### Efficacy of a Dual Vaccine Comprising PapMV-Porin and a Commercial Influenza Vaccine

In this Example, the candidate vaccine described in Example 2 (comprising PapMV VLPs and OmpC combined in a 1:1 w/w ratio (10 μg of each)) was combined with the Fluviral® influenza vaccine and the ability of the dual vaccine to protect mice against an influenza challenge was measured. Fluviral® is a commercially available trivalent, inactivated split-virion vaccine prepared in eggs (ID Biomedical Corporation, Date of Approval: May 2, 2007, GlaxoSmithKline Biologicals North America, Quebec City, QC, Canada) and comprises the influenza strains: A/Solomon Islands/3/2006, A/Wisconsin/67/2005, B/Malaysia/2506/2004.

### Experimental Design:

3 groups of 5 Balb/C mice mice were inoculated subcutaneously with the following:
Group I: Fluviral® ⅕ of human dose
Group II: Fluviral® ⅕ of human dose+PapMV CPsm 3 μg+OmpC 3 μg
Group III: Fluviral® ⅕ of human dose+PapMV CPsm 30 μg+OmpC 30 μg

Animals were inoculated at day 0, and bleedings were done at day 0 and day 14. ELISAs were performed to measure the total amount of IgG, the amount of IgG1 and the amount of IgG2a raised against Fluviral® or the purified S. typhi NP protein. At day 28, the mice were challenged with 4LD₅₀ of the heterologous influenza strain A/WSN/33 (i.e. 4000 pfu/mouse in a volume of 50 μl). By “heterologous” it is meant a strain of influenza against which the commercial Fluviral® vaccine can not induce protection in vaccinated mice.

### Results:

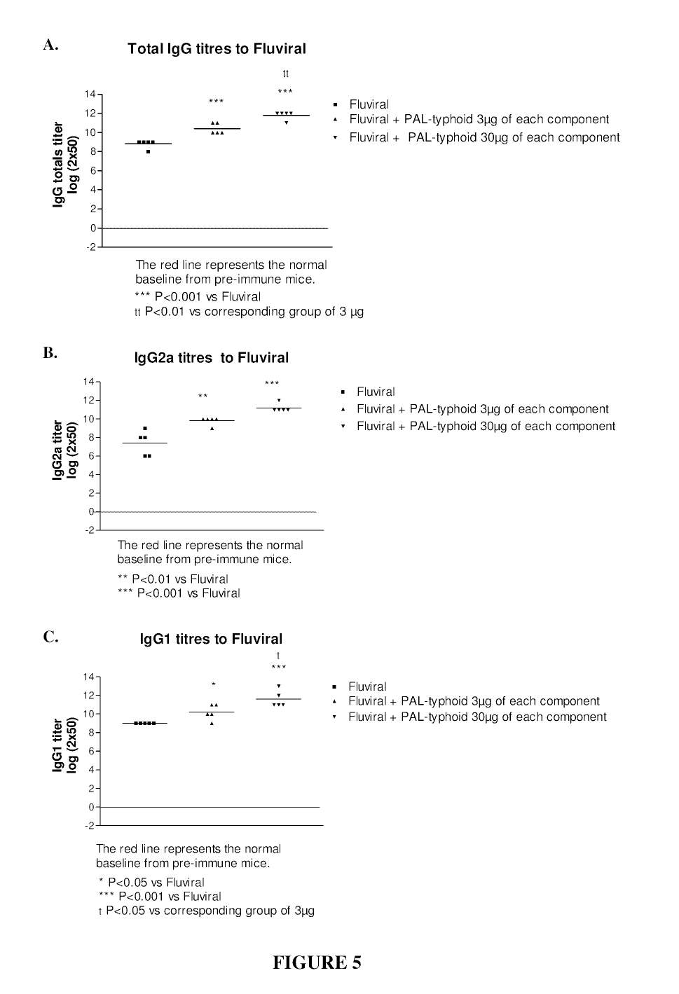

The results are presented in FIGS. 5-8 and demonstrate that the candidate dual vaccine resulted in an improved immune response in Balb/C mice.

The addition of only 3 μg of PapMV VLP-OmpC improved the immune response as measured by total IgG to Fluviral® by 4-fold after only one injection (FIG. 5A). When 30 μg of PapMV VLP-OmpC was used, an increase of 8-fold in the total amount of IgG was observed (FIG. 5A).

The amount of IgG2a directed to the Fluviral® proteins was also measured. The IgG class switch that induces the production of IgG2a is indicative of the stimulation of a TH1 response in mice. Both adjuvant regimens were seen to induce a significant improvement in the immune response to Fluviral® by a factor of 8- or 16-fold when 3 μg or 30 μg of PapMV VLP-OmpC, respectively, were mixed with Fluviral® (FIG. 5B). This is a striking improvement over immunisation with Fluviral® alone.

Interestingly, the amount of IgG1 also increased significantly when PapMV VLP-OmpC was added to Fluviral® (see FIG. 5C). IgG1 is a marker for the TH2 response. The addition of 3 μg or 30 μg of PapMV VLP-OmpC increased the amount of IgG1 to Fluviral® produced by a factor of 2- or 4-fold, respectively. Together, the induction of IgG2a and IgG1 suggests that both TH1 and TH2 responses are induced by the PapMV VLP-OmpC vaccine.

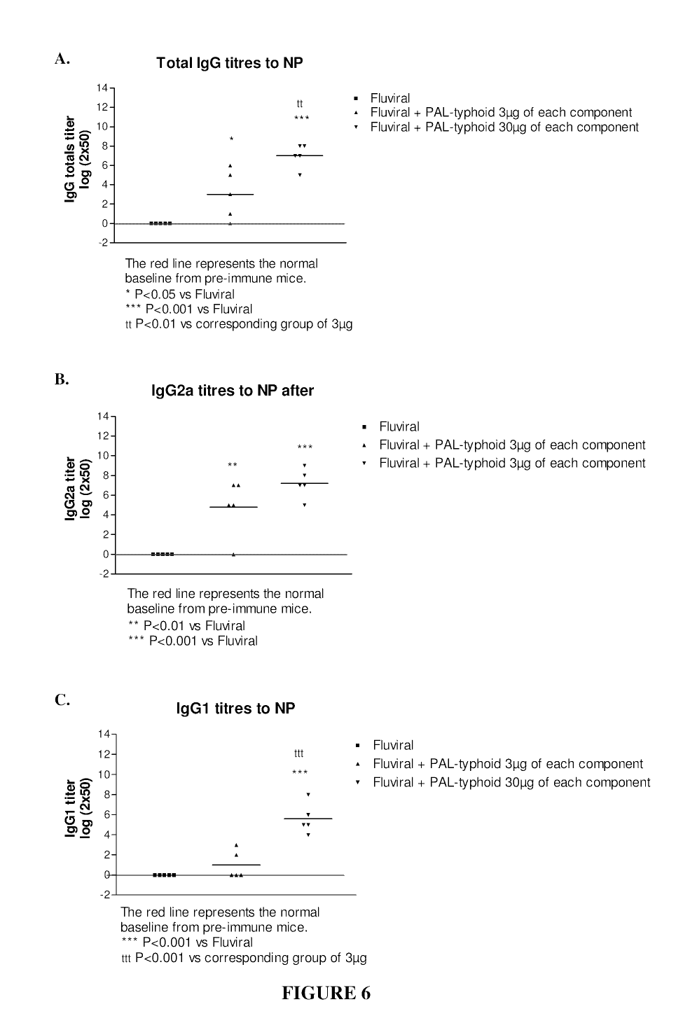

The candidate dual vaccine also improved the immune response to the influenza NP protein, which is a conserved protein through all the strains of the influenza virus. The immune response directed to this protein is negligible in mice vaccinated with the commercial Fluviral® vaccine (see FIG. 6). Addition of PapMV VLP-OmpC to the Fluviral® vaccine improved considerably the immune response to NP in vaccinated mice and a significant amount of antibodies directed to this highly conserved target was measured. The increment of the total IgG (FIG. 6A), as well as of IgG2a (FIG. 6B) and IgG1 (FIG. 6C) were directly proportional to the amount of PapMV VLP-OmpC added to the Fluviral® vaccine. Titres of IgG2a as high as 1/12800 were obtained when 30 μg of the PapMV VLP-OmpC were used. This result suggests that a CTL response directed to NP was also triggered in mice immunized with Fluviral® adjuvanted with PapMV VLP-OmpC.

The outstanding improvement of the TH1 immune response to the Fluviral® and NP proteins when the PapMV VLP-OmpC was combined with Fluviral® suggested that the adjuvanted Fluviral® vaccine could provide protection to a heterologous strain of influenza through the triggering of a CTL response to a highly conserved epitope such as the NP protein.

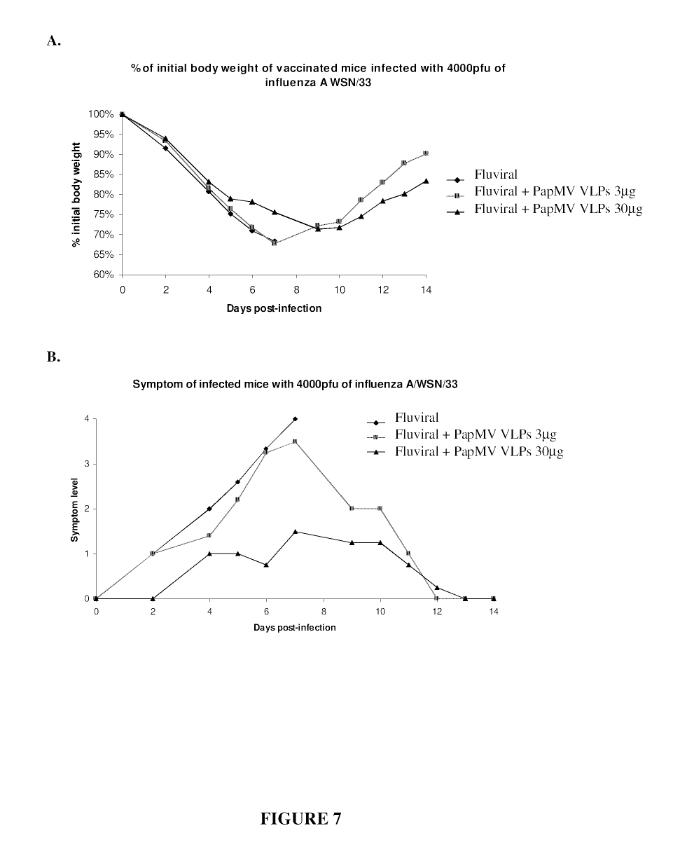

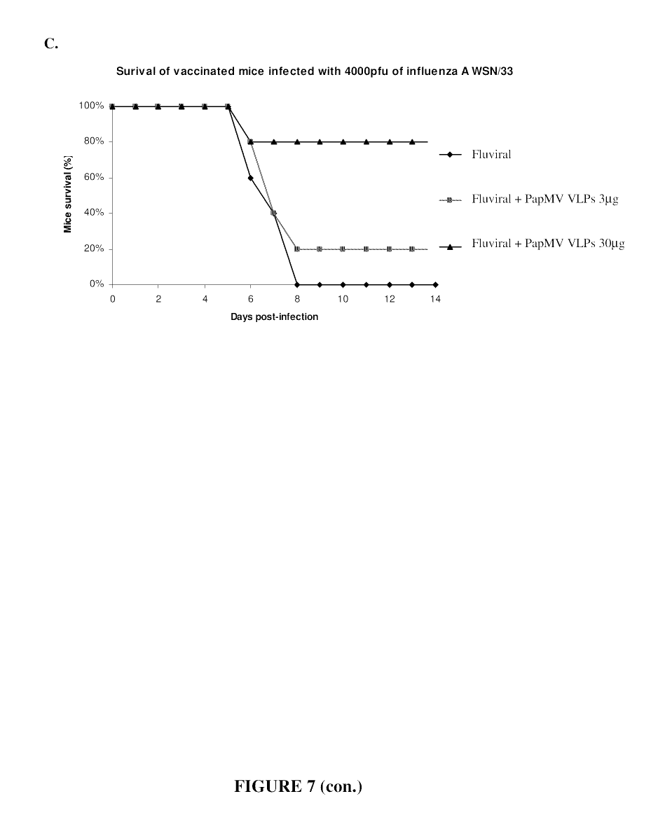

To verify this assumption, the vaccinated mice were challenged with a heterologous strain (WSN/33) of influenza. Mice vaccinated with one fifth of the human dose of Fluviral® were subsequently challenged with 4,000 pfu (plaque forming units) (equivalent to 4LD₅₀) of WSN/33. All the mice treated with this vaccination schedule showed a rapid decrease in body weight (FIG. 7A), as well as severe symptoms (FIG. 7B; see Table 5 below for symptoms legend) and finally all mice in this group died of the infection (FIG. 7C). However, the mice vaccinated with Fluviral® in combination with PapMV VLP-OmpC showed less body weight loss (FIG. 7A), less severe symptoms (FIG. 7B) and an overall survival of 80% when 30 μg of PapMV VLP-OmpC was added to Fluviral® (FIG. 7C).

TABLE 5
Symptoms legend for FIG. 7B

| Rating | Symptoms |
|---|---|
| 0 | No symptoms |
| 1 | Lightly spiked fur; Lightly |
| | curved back |
| 2 | Spiked fur; Curved back |
| 3 | Spiked fur; Curved back; |
| | Difficulty moving; Light |
| | dehydration |
| 4 | Spiked fur; Curved back; |
| | Difficulty moving; Severe |
| | dehydration/thin; Lifeless/ |
| | closed eyes; Ocular secretion |
| | This level leads to euthanasia |

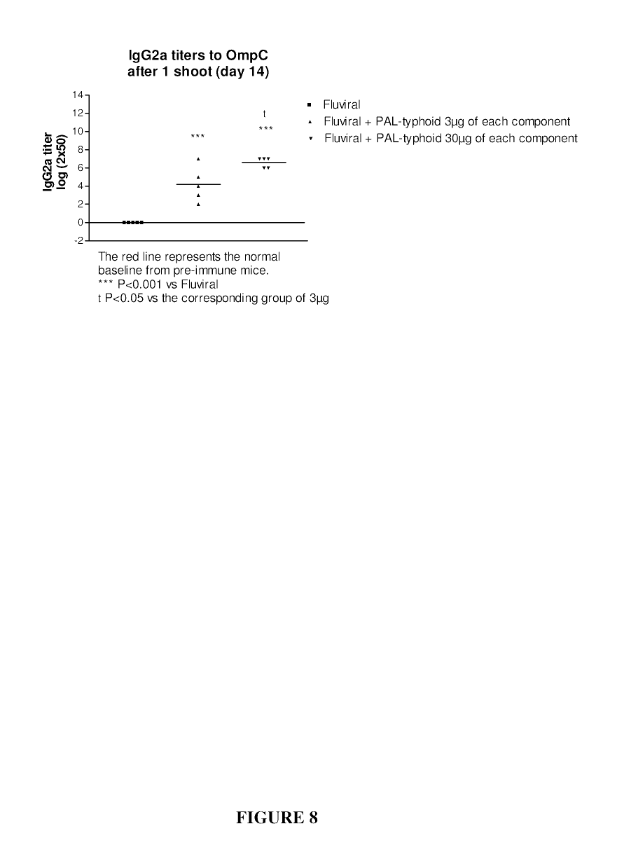

The combination of PapMV VLPs with OmpC has been shown to improve the protection to a S. typhi challenge (see Example 2 and FIG. 4). Mice vaccinated with PapMV VLP-OmpC also showed a high level of antibodies specific to OmpC. It appears likely therefore that high levels of antibodies correlate with protection and it is anticipated that mice showing a high level of at least IgG2a will be protected to a S. typhi challenge. To verify that the Fluviral® proteins do not interfere with the immune response to OmpC, sera from the vaccinated mice was tested by ELISA for antibodies directed to OmpC. High levels of IgG2a were produced to OmpC in the mice vaccinated with PapMV VLP-OmpC in combination with Fluviral® (FIG. 8), which strongly suggests that mice vaccinated with the candidate dual vaccine will be protected against both influenza and S. typhi infection.

### Concluding Remarks

The results described above demonstrate that the formulation of a dual vaccine comprising PapMV VLP-OmpC and Fluviral® is immunogenic and induces production of antibodies to OmpC and to the Fluviral® proteins, supporting the rationale that the dual vaccine will provide protection against infection with both pathogens: influenza and Salmonella typhi. In addition, an improved immune response to the Fluviral® proteins was observed that goes beyond what is normally observed when Fluviral® in used alone. Therefore, the PapMV VLP-OmpC plays the role of an adjuvant to the Fluviral® proteins. A balanced TH1 and TH2 response was induced, symptoms due to the infection were attenuated and a protection of 80% to a heterologous influenza strain was measured in mice vaccinated with the dual vaccine. This result suggests that a CTL response to a highly conserved epitope of influenza is triggered, which provides protection to a heterologous strain of influenza.

To confirm that the PapMV VLP component of the PapMV-OmpC alone can play the role of an adjuvant to the Fluviral® vaccine, a similar experiment was conducted in which PapMV VLPs were used alone as an adjuvant to the Fluviral® vaccine (see Example 4, below). PapMV VLPs were shown to be an excellent adjuvant of Fluviral® and improved significantly the immune response to the Fluviral® proteins as well as to the highly conserved NP protein. Challenge of the vaccinated mice with the heterologous strain WSN/33 confirmed that PapMV VLPs are also able to trigger a TH1 response that provides protection to 40% of the infected mice.

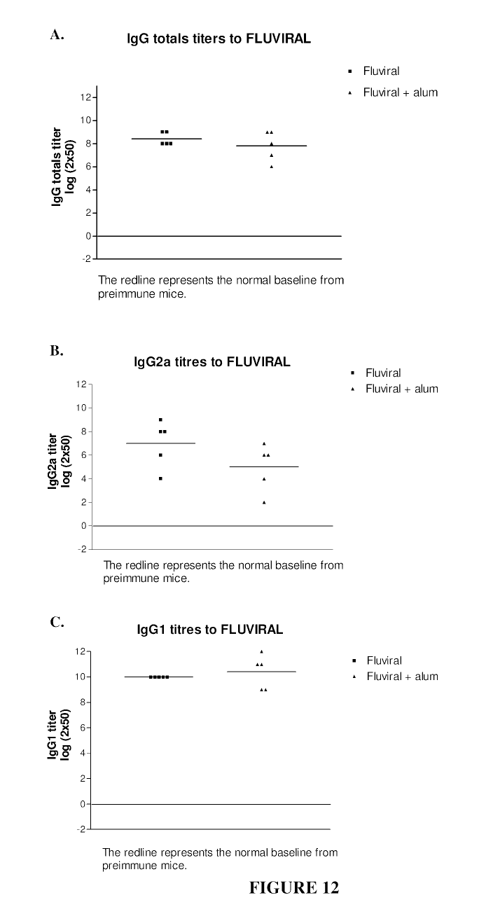

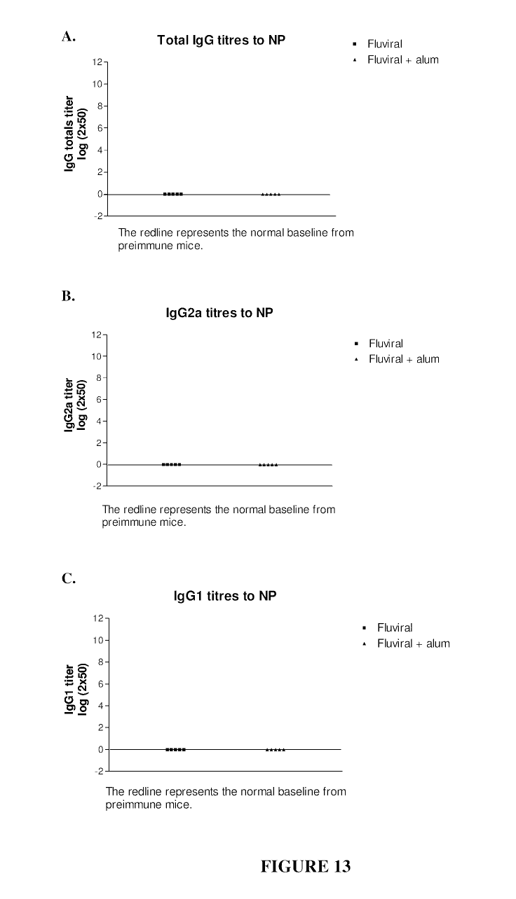

Both formulations, i.e. Fluviral®+PapMV VLPs and the candidate dual vaccine (Fluviral®+PapMV-OmpC) were superior to alum, the only adjuvant used in North America in humans. Alum was unable to increase the immune response to the Fluviral® proteins (FIG. 12) or to induce an immune response to the NP protein (FIG. 13).

It is likely that Fluviral® formulations containing PapMV VLPs or PapMV-OmpC will be able to protect against infection to various strains of influenza, including the avian flu, through the ability of these formulations to trigger an immune response to the highly conserved NP protein.

### Example 4

### Use of PapMV VLPs Alone as an Adjuvant of the Fluviral® Vaccine #1

To confirm that PapMV VLPs alone can improve the immune response to the Fluviral® proteins and provide protection to a heterologous strain of influenza, a similar experiment to that described in Example 3 was conducted, with the exception that PapMV VLPs alone were used to adjuvant the Fluviral® vaccine. Results of a similar experiment conducted with PapMV VLPs comprising a different version of the PapMV coat protein is provided in Example 12 below.

### Experimental Design:

3 groups of 5 Balb/C mice mice were inoculated subcutaneously with the following:
Group I: Fluviral® ⅕ of human dose
Group II: Fluviral® ⅕ of human dose+PapMV VLPs 3 μg
Group III: Fluviral® ⅕ of human dose+PapMV VLPs 30 μg

Animals were inoculated at day 0, and bleedings were done at day 0 and day 14. ELISAs were performed to measure the total amount of IgG, the amount of IgG1 and the amount of IgG2a raised against Fluviral® or the purified S. typhi NP protein. At day 28, the mice were challenged with 4LD₅₀ of the heterologous influenza strain A/WSN/33 (=4000 pfu/mouse in a volume of 50 μl).

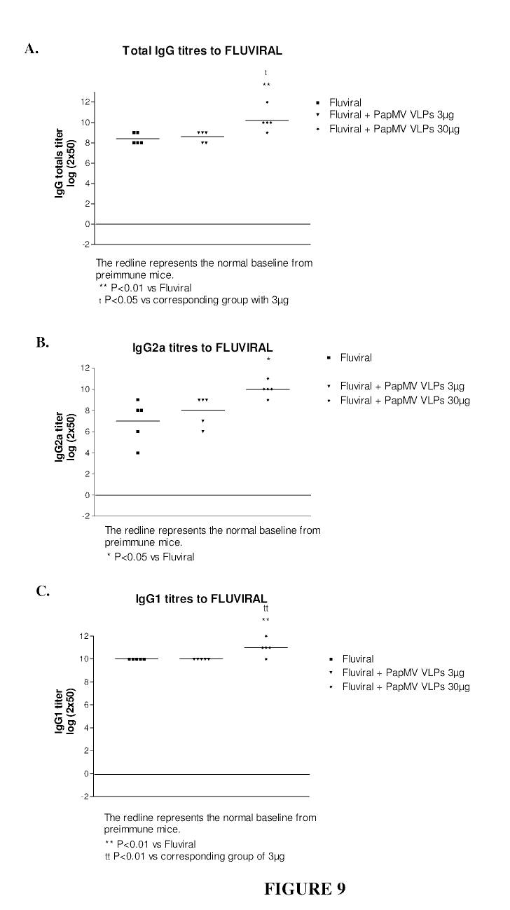

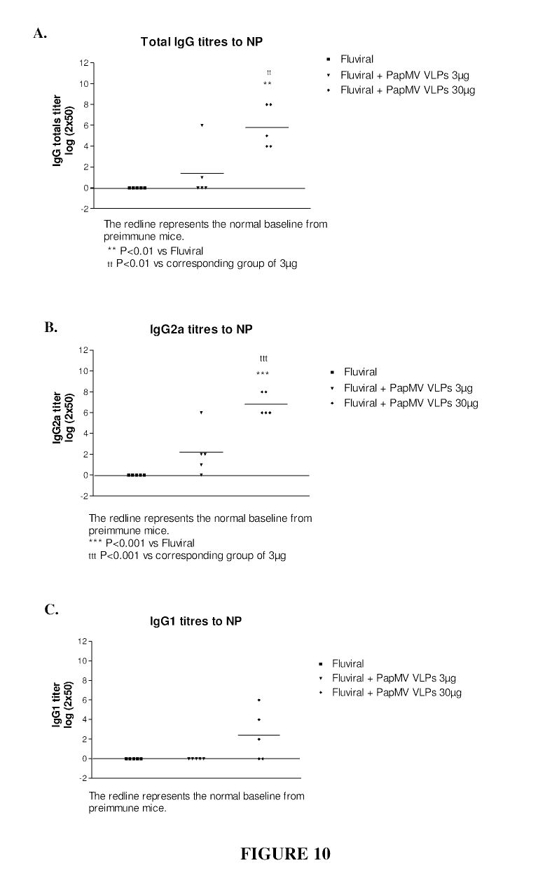

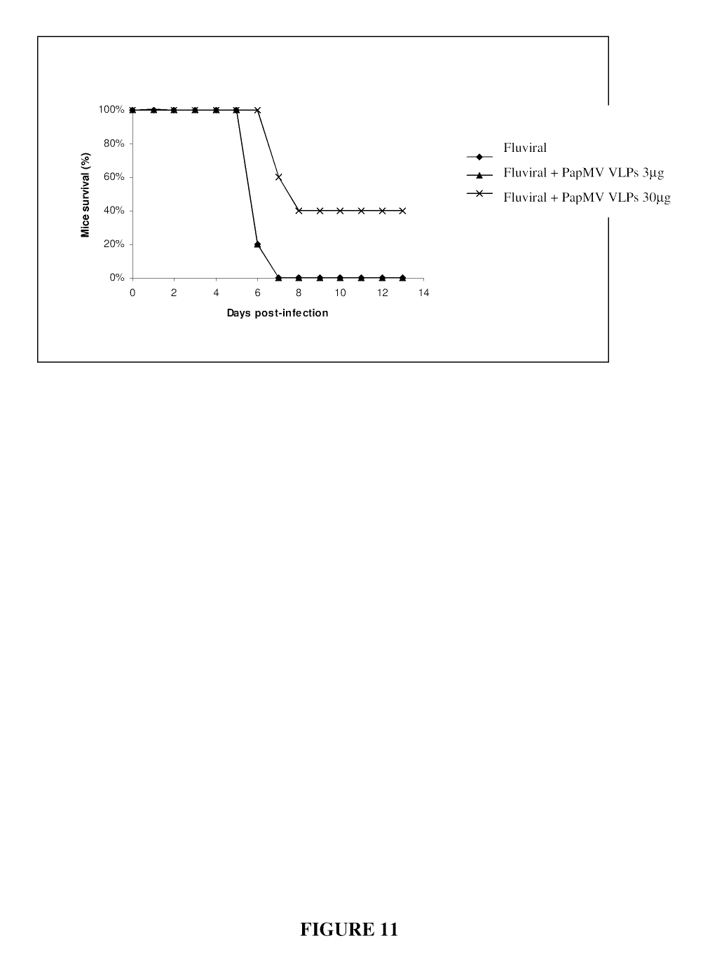

The results are presented in FIGS. 9 and 10, and show that the total IgG, the IgG1 and the IgG2a response to the Fluviral® proteins (FIGS. 9A-C) and to the NP protein (FIGS. 10A-C) increased significantly with increasing amounts of PapMV VLPs. This improvement correlated with a protection of 40% in the mice vaccinated with Fluviral®+30 μg of PapMV VLPs to the challenge with the WSN/33 strain of influenza (FIG. 11).

### Example 5

### Preparation of PapMV VLPs Comprising Affinity Peptides for OmpC or OmpF

### Selection of Affinity Peptides

Specific peptides against purified OmpC and OmpF were selected using the Ph.D-7 Phage Display Peptide Library Kit (New England Biolabs, Inc.). The protocol followed was an in vitro selection process known as “panning,” which was conducted according to the manufacturer's protocol. Briefly, 2×10¹¹ phage were added to 10 ug of purified OmpC or OmpF bound to the base of the wells of an ELISA plate and the contents of the well gently mixed at room temperature for 1 hour. Unbound phage were eluted with 1 ml of 200 mM Glycine-HCl (pH 2.2), by incubating for 10 min at room temperature. To neutralize the supernatant, and to avoid killing the phage, 150 μl of 1M Tris-HCl (pH 9.1) was added. The eluted phage were then amplified and taken through additional binding/amplification cycles to enrich the pool in favour of binding sequences. The wash buffer contained 0.1% of Tween 20 for the first round of panning and was increased to 0.5% for subsequent rounds. Selected phage were amplified in E. coli ER2738 between each panning round. The cycle was repeated 3 times to select those peptides with the highest affinity for the respective porin proteins. The peptides thus identified are shown in Table 6.

TABLE 6
Sequence and Frequency of Occurrence
of OmpC and OmpF Affinity Peptides

| Target Protein | Sequence of Peptide | Frequency | SEQ ID NO |
|---|---|---|---|
| OmpC | SLSLIQT | 1/8 | 10 |
| OmpC | EAKGLIR | 6/8 | 11 |
| OmpC | TATYLLD | 1/8 | 12 |
| OmpF | FHENWPS | 3/5 | 13 |
| OmpF | FHEFWPT | 2/5 | 14 |

### Engineering, Expression and Purification of the High Avidity PapMV VLPs Fused to the Selected Affinity Peptides

One affinity peptide was selected from those identified in the above panning process for each porin, OmpC and OmpF. The corresponding DNA sequence was cloned at the C-terminus of PapMV coat protein (CP). PapMV CP CPΔN5 (Tremblay, M-H., et al., 2006, FEBS J., 273:14-25) was used as the template. The sequence encoding each selected peptide was introduced using PCR and cloned into the pET-3D expression vector (Stratagene, La Jolla, Calif.). In brief, the forward primer (SEQ ID NO:34; below) and the primer PapOmpC (SEQ ID NO:35; below) were used in the PCR reaction with the PapMV CP gene PapMV CP CPΔN5 as template.

Forward Primer:
[SEQ ID NO: 34]
5′-ATCGCCATGGCATCCACACCCAACATAGCCTTCCCCGCCATCACC-
3′
PapOmpC (Reverse Primer):
[SEQ ID NO: 35]
3′-GGTTAAGGAAGGTGGGGGGCTTCTCCGCTTCCCCAACTAAGCATGG
TAGTGGTAGTGGTAATCATTCCTAGGTGAC-5′

The resulting PCR fragment harbours a fusion of the peptide EAKGLIR at the C-terminus of the PapMV CP. Using the same approach, the forward primer (SEQ ID NO:34) and the primer PapOmpF (SEQ ID NO:36; below) were used to introduce a fusion of the peptide FHENWPS at the C-terminus of the PapMV CP by PCR.

PapOmpF (Reverse Primer):
[SEQ ID NO: 36]
3′-GGTTAAGGAAGGTGGGGGGCTTAAAGTACTCTTAACCGGAAGCGTG
GTAGTGGTAGTGGTAATCATTCCTAGGTGAC-5′.

The two respective PCR fragments were digested with the restriction enzymes NcoI and BamHI and cloned into the pET 3-D vector digested with the same enzymes. Clones were sequenced to verify that the peptides were in frame with the PapMV CP.

Engineered PapMV CPs comprising the affinity peptide were expressed in E. coli BL21 RIL as described previously (Tremblay, M-H., et al., 2006, FEBS J., 273:14-25; Secundino et al., 2006, Immunology 117:59). Briefly, the bacteria were lysed through a French Press and loaded onto a Ni²⁺ column, washed with 10 mM Tris-HCl; 50 mM Imidazole; 0.5% Triton X100; pH8, then with 10 mM Tris-HCl, 50 mM Imidazole, 1% Zwittergent, pH8 to remove endotoxin contamination. The eluted proteins were subjected to high-speed centrifugation (100 000 g) for 120 min in a Beckman 50.2 Ti rotor. The VLP pellet was resuspended in endotoxin-free PBS (Sigma). Proteins were filtered using 0.45 μM filters before use. The purity of the proteins was determined by SDS-PAGE. The amount of protein was evaluated using the BCA protein kit (Pierce). The level of LPS in the purified protein was evaluated with the Limulus test according to the manufacturer's instructions (Cambrex) and was below 0.005 endotoxin units (EU)/μg of protein.

The sequences of the two PapMV coat proteins are shown in FIG. 3 (SEQ ID NO:6—PapMV coat protein including the OmpC affinity peptide (FIG. 3A), and SEQ ID NO:7—PapMV coat protein including the OmpF affinity peptide (FIG. 3B)). Two amino acid differences were observed in the coat protein sequence of the PapMV coat protein including the OmpC affinity peptide as compared to the wild-type (in bold and underlined in FIG. 3A), which were likely introduced during the PCR reaction.

### ELISA

For each experiment, 10 μg of the respective target protein (OmpC or OmpF) was used to coat an ELISA plate. Increasing amounts of the PapMV VLPS were used for the binding assay. The affinity of the VLPs for their target was revealed using polyclonal mouse antibodies directed to the PapMV CP and a secondary anti-mouse antibody coupled to peroxidase.

### Results

Phage display was used to select specific peptides binding to OmpC or OmpF. Eight phage that bound to OmpC and five phage that bound to OmpF were sequenced. The sequences and frequency of occurrence of these peptides is show in Table 6. The peptide EAKGLIR [SEQ ID NO:11] showed the highest frequency and, therefore, was selected as the affinity peptide to OmpC. The peptide FHENWPS [SEQ ID NO:12] was the most frequent in the OmpF screening and was, therefore, selected as the affinity peptide to OmpF. Interestingly, both affinity peptides to OmpF were homologous since 5 out of 7 amino acids were identical and found in the same position in the affinity peptides.

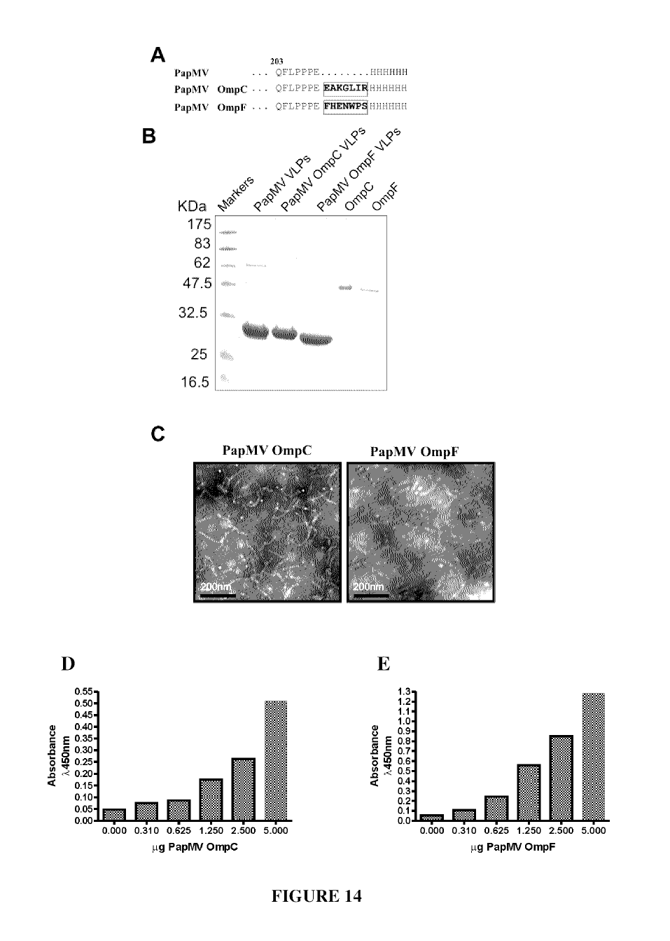

The peptide sequences EAKGLIR [SEQ ID NO:11] and FHENWPS [SEQ ID NO:12] were fused at the C-terminus of the PapMV coat protein (FIG. 14A). The fusion peptide was followed by a 6×H tag to facilitate the purification process (Tremblay, M-H., et al., 2006, ibid.). FIG. 14B shows an SDS-PAGE gel of the recombinant proteins. Electron microscopy (EM) observations confirmed that the addition of the peptides at the C-terminus of the PapMV CP did not affect the ability of the protein to self-assemble into VLPs (FIG. 14C). The lengths of the VLPs are variable, with a size range of 201±80 nm. A 201 nm length protein represents 560 copies of the CP presenting the peptide in a repetitive fashion.

The high avidity of each of the PapMV VLPs to their respective antigen was shown by an ELISA-type binding assay. For both VLPs, binding to their respective antigen was clearly demonstrated and increased with the amount of VLPs used in the assay (FIGS. 14D & E). It was, therefore, assumed that PapMV VLPs will bind to the cognate antigen to form a complex when mixed in a 1:1 ratio in solution.

### Example 6

### Immunization Against Salmonella typhi with High Avidity PapMV VLPs

### Mice

Female BALB/c mice 6-8 weeks old (Harlan, Mexico or Charles River, Canada) were used and kept in the animal facilities of the Experimental Medicine Department, Medicine Faculty, National Autonomous University of Mexico (UNAM) or at the animal facilities from Centre Hospitalier de l'Universite Laval.

### Challenge Assay

Mice (10 per group) were immunized intraperitoneally (i.p) (day 0) in the absence of external adjuvant with 10 μg OmpC, 10 μg OmpC+10 μg PapOmpC, 10 μg OmpF, 10 μg OmpF+10 μg PapOmpF, 10 μg PapOmpC, 10 μg PapOmpF or saline (SSI). On day 15, mice received a boost i.p with 10 μg OmpC or 10 μg OmpF, respectively, without adjuvant. On day 25 or 140 the mice were challenged i.p with 100 or 500 LD₅₀ of Salmonella typhi (ATCC 9993) resuspended in 500 μL TE buffer (50 mM Tris, pH 7.2, 5 mM EDTA) containing 5% gastric mucin (Sigma). Protection was defined as the percentage survival 10 days following the challenge. 1 LD₅₀ was determined at 90 000 CFU.

### Immunizations

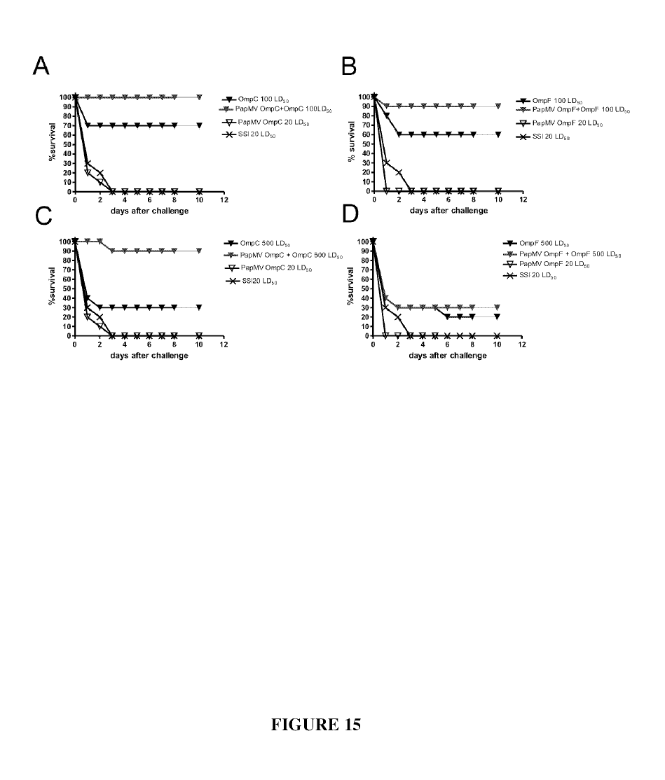

Groups of 5 mice were immunized (day 0) intraperitoneally (i.p) in the absence of external adjuvant with 10 μg OmpC, 10 μg OmpC+10 μg PapOmpC, 10 μg PapOmpC or isotonic saline solution (ISS). On day 15, mice received a boost i.p with 10 μg OmpC without adjuvant. Blood samples were collected from the jugular vein at various times as indicated in FIGS. 15 & 16. Individual serum samples were stored at −20° C. until analysis.

### Elisa

High binding 96-well polystyrene plates (Nunc) were coated with 10 μg/mL of OmpC in 0.1M carbonate-bicarbonate buffer ph 9.5. Plates were incubated for 1 hour at 37° C. followed by overnight at 4° C. Plates were washed four times with distilled H2O-0.1% Tween 20. Non-specific binding was blocked with blocking buffer (PBS pH 7.4-2% BSA (Sigma)) for 1 hour at 37° C. After washing, pooled mice sera were diluted 1:40 in blocking buffer and two-fold serial dilutions were added to the wells. Plates were incubated 1.5 hours at 37° C., followed by four washes. HRP-conjugated goat anti-mouse IgG1, IgG2a, IgG2b (Jackson Immunochemicals) or IgG3 (Rockland) (1:10 000) was added and incubated 1 hour at 37° C. followed by four washes. As the detection system, TMB peroxidase substrate (Fitzgerald) was used. After incubation in the dark for 10 minutes at 37° C. the reaction was stopped with 2.5N H₂SO₄ and the absorbance was determined at 450 nm using an automatic ELISA plate reader. Antibody titers are given as −log₂ dilution X 40. Positive titers were defined as 3 SD above the mean values of the negative controls.

### Passive Immunization and Challenge

Groups of 5 mice were immunized i.p (day 0) in the absence of external adjuvant with 10 μg OmpC, 10 μg OmpC+10 μg PapMV OmpC, 10 μg PapMV OmpC or isotonic saline solution (SSI). On day 15, mice received a boost i.p with 10 μg OmpC without adjuvant. Cardiac puncture was performed on day 23 and serum samples from each group were pooled and stored at −20° C. Naïve mice (5 per group) received i.p 200 μL of the pooled complement-inactivated immune sera. Three hours after transference mice were challenged with 100 LD₅₀ of S. typhi resuspended in mucin, as described above. Protection was defined as the percentage survival 10 days following the challenge.

### Results

The purified proteins OmpC and OmpF were previously shown to provide protection against S. typhi challenge in mice, with OmpC alone providing 60% protection against 100LD₅₀ (Secundino et al., 2006, Immunology 117:59). To improve the immunogenicity of each of OmpC and OmpF, vaccine formulations comprising PapMV OmpC VLPs+OmpC and PapMV OmpF VLPs+OmpF, were tested in mice for their capacity to protect mice toward 100 and 500 LD₅₀ of S. typhi and the results compared with those obtained with mice immunized with OmpC or OmpF alone. The ratio between the PapMV VLPs and their respective porin was maintained at 1:1.

Addition of PapMV OmpC VLPs to OmpC improved the protection capacity of OmpC from 70% to 100% with a challenge of 100LD₅₀ of S. typhi (FIG. 15A). This improvement of the protection efficacy was even greater when mice were challenged with 500LD₅₀, with the protection observed increasing from 30% to 90% when OmpC was combined with PapMV OmpC VLPs (FIG. 15C). Similarly, PapMV OmpF VLPs improved the protection capacity of OmpF from 60% to 90% with a challenge of 100LD₅₀ of S. typhi (FIG. 15B), however, only a minor difference was observed when the challenge was conducted with 500LD₅₀ of S. typhi (FIG. 15D). The results suggest that OmpC is a better antigen than OmpF for protection against S. typhi challenge. In both cases, PapMV VLPs considerably improved the protective capacity of the porins.

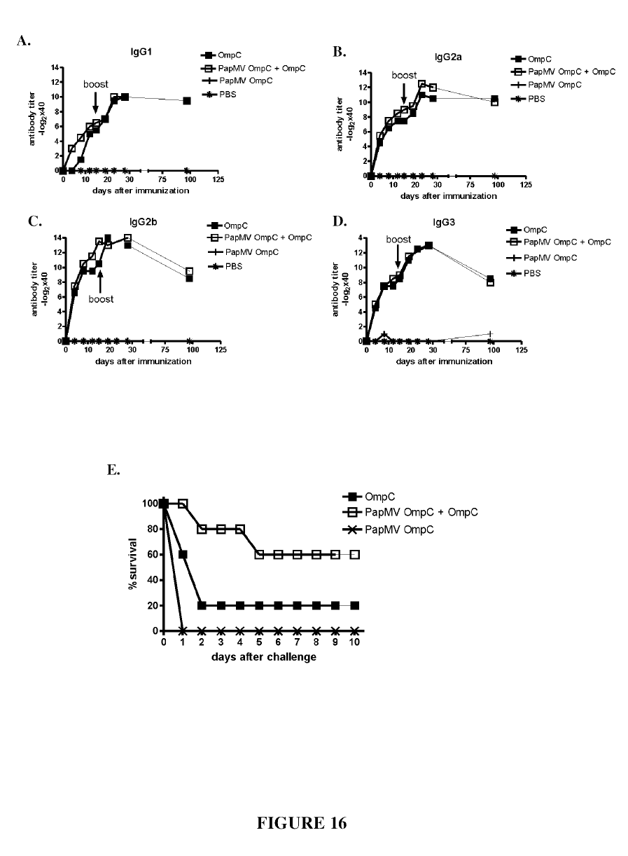

To determine if PapMV VLPs improved antibody titers to OmpC, the IgG titers of mice vaccinated with 10 μg OmpC or with the conjugated vaccine containing 10 μg OmpC and 10 μg PapMV OmpC VLPs were measured. No significant difference was found in the titers of the different IgG isotypes IgG1, IgG2a, IgG2b and IgG3 with either treatment (FIG. 16A-D) suggesting that the improvement of the protection observed with PapMV VLPs may be related to an improvement in the CTL response and/or in the binding efficacy of the antibodies in neutralising S. typhi infection, rather than an increase of production of antibodies per se.

To evaluate the memory response of the vaccine preparation comprising the PapMV VLPs in combination with OmpC, mice were immunized twice at two-week intervals with either OmpC alone, or with the vaccine preparation comprising PapMV OmpC VLPs and OmpC, followed with a boost at day 15 with OmpC alone. At day 140, the mice were challenged with 100LD₅₀ of S. typhi. The results clearly show that priming with the vaccine preparation comprising PapMV OmpC VLPs and OmpC significantly improved (by 3 times) the protection capacity of vaccinated mice (FIG. 16E). This experiment thus demonstrates that PapMV VLPs not only improve the protection of mice to S. typhi challenge, but also provide a better memory response.

### Example 7

### Protective Capacity against S. typhi of a Combination of PapMV and OmpC

### Purification of PapMV

PapMV was purified by differential centrifugation from infected papaya leaves that showed mosaic symptoms. Infected leaves (100 g) were ground in 100 mL 50 mM Tris-HCl (pH 8.0) containing 10 mM EDTA in a commercial blender. The ground leaves were filtered through cheesecloth, 1% of Triton X-100 was added to the filtrate, and the filtrate was stirred gently for 10 min. Chloroform was added drop by drop to a volume equivalent to one-quarter of the volume of the filtrate. The solution was stirred for an additional 30 min at 4° C. and centrifuged for 20 min at 10 000 g to remove the precipitate. The supernatant was subjected to high-speed (100 000 g) centrifugation for 120 min. The viral pellet was suspended and subjected to another high-speed centrifugation through a sucrose cushion (30% sucrose) at 100 000 g for 3.5 h. The final viral pellet was suspended in 10 mL of 50 mM Tris (pH 8.0). If color persisted, an additional clarification with chloroform was performed. The purified virus was collected by ultracentrifugation.

### Protection Assay

BALB/c mice (groups of 10) were immunized i.p. on day 0 with 10 μg of OmpC or 10 μg of OmpC that had been incubated previously for 1 h at 4° C. with 30 μg of PapMV. A boost on day 15 was performed with 10 □g of OmpC alone. Control mice were injected with saline only. On day 21, the mice were challenged with 100 and 500 LD₅₀ of S. typhi (STYC302 ΔompF strain) suspended in 5% mucin (as described above) and the survival rate was monitored for 10 days after the challenge, as described previously (Isibasi et al., 1992, Vaccine 10:811-813; Isibasi et al., 1988, Infect. Immun. 56:2953-2959).

### Results

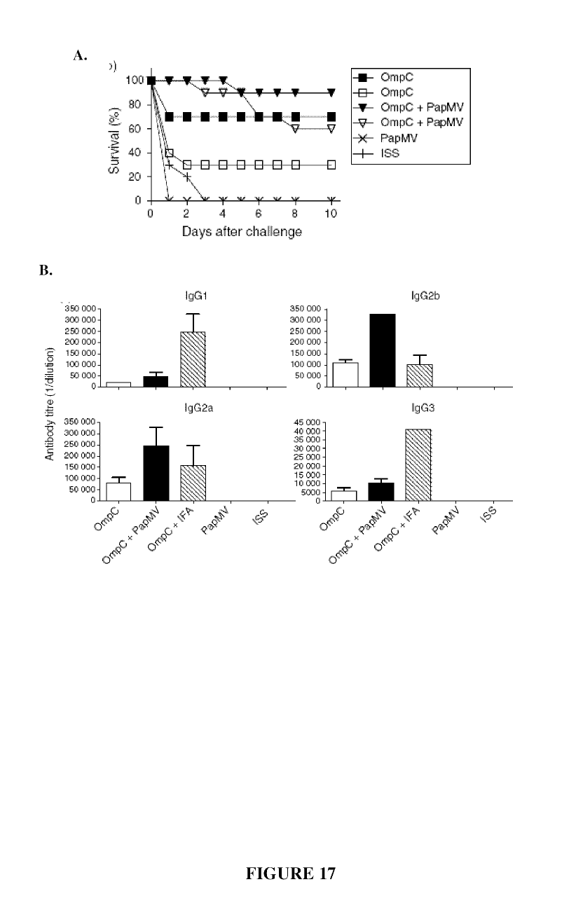

The adjuvant capacity of PapMV virus isolated from infected plants in increasing the protection provided by OmpC was tested. A survival rate 30% higher after challenge with either 100LD₅₀ or 500LD₅₀ of S. typhi was observed when OmpC mixed with PapMV purified virus was employed as compared to OmpC alone (FIG. 17A). No protection was observed in mice immunized only with PapMV on days 0 and 15 and challenged on day 21 (FIG. 17A), nor in mice immunized with PapMV and challenged 24 hr later. These data reject the idea that the protection observed is the result of enhanced inflammation induced by PapMV. To test if increased protection correlates with an increase in the antibody titre specific for OmpC, the PapMV adjuvant effect on the OmpC-specific antibody titres was measured. PapMV co-immunization with OmpC induced an increase in the anti-OmpC IgG1, IgG2a, IgG2b and IgG3 titres (FIG. 17B). These results further corroborate that the adjuvant properties of PapMV potentiate both innate and adaptive immune responses elicited by OmpC to achieve protection against S. typhi challenge. Co-administration of PapMV and S. typhi OmpC porin can thus be seen to increase the protective capacity against S. typhi challenge.

The results of the experiments outlined in this Example and Example 6 indicate that PapMV has intrinsic adjuvant properties that can induce the switch of antigen-specific immunoglobulins, provide a sustained long lasting antibody response to model antigens, and increase the protective capacity of OmpC or OmpF alone. These data indicate that PapMV and PapMV VLPs potentiate the translation of innate and adaptive immune responses elicited by OmpC porin into protection against S. typhi challenge.

### Example 8

### Protective Capacity of PapMV VLPs with S. typhi OmpF

The following proteins (as described in the preceding Examples) were used in this experiment:
PapMV CPΔN5 VLPs (“PapMV”).
PapMV OmpF VLPs that comprise the affinity peptide for S. typhi OmpF
OmpF

Balb/C mice, 10 mice per group, were immunized intraperitonally (I.P.) at day 0 as follows:

### Group 1: 10 μg OmpF

### Group 2: 10 μg OmpF+10 μg PapMV

### Group 3: 10 μg OmpF+10 μg PapMV OmpF

### Group 4: 10 μg PapMV

### Group 5: 10 μg PapMV OmpF

A second immunization (Boost) was performed at day 15 using 10 μg OmpF in groups 1, 2 and 3. Group 4 was boosted with 10 μg PapMV, and group 5 was boosted with 10 μg PapMV OmpF. Challenge with S. typhi was performed on day 21. It was established experimentally that 90 000 CFU of S. typhi in mucin correspond to 1 LD₅₀. All mice were sacrificed at day 31.

### Results

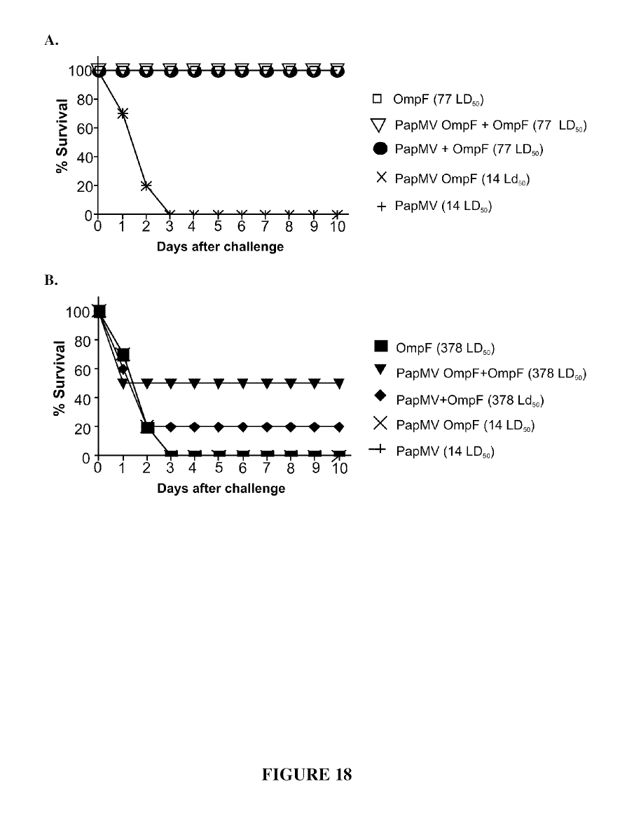

At 77 LD50 (FIG. 18A), all preparations containing OmpF provided 100% protection. As expected, the mice vaccinated with preparations that did not contain OmpF all died within a few days of challenge with doses as low as 14LD₅₀. A difference was observed, however, between the OmpF containing preparation when the mice were challenged with a higher dose of S. typhi (378LD₅₀). As shown in FIG. 18B, the most effective preparation at this dose was the combination of PapMV OmpF+OmpF in the first immunisation (Group 3) and a boost with OmpF alone. It is likely that the high avidity of the PapMV OmpF VLPs for their target, OmpF, improved the protection capacity as compared to the use of PapMV alone as an adjuvant to OmpF.

This data is consistent with that shown in Example 7 and confirms that PapMV OmpF improves the protection capacity of OmpF to a challenge with S. typhi. Furthermore, the data show that PapMV OmpF is a better adjuvant of OmpF than PapMV VLPs without an affinity peptide. As noted above, it is likely that a stronger binding of PapMV OmpF VLPs to OmpF promotes formation of a VLP-OmpF complex, which in turn maximises the adjuvant capacity of the VLP molecule.

### Example 9

### Protective Capacity of PapMV VLPs with S. typhi OmpC

The following proteins, as described in the preceding Examples, were used in this experiment:
PapMV CPΔN5 VLPs (“PapMV”)
OmpC

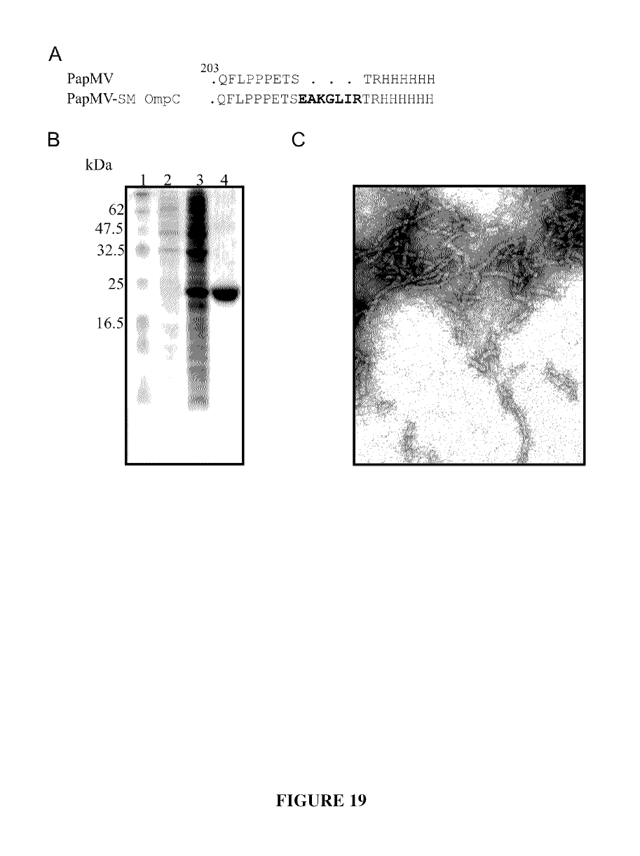

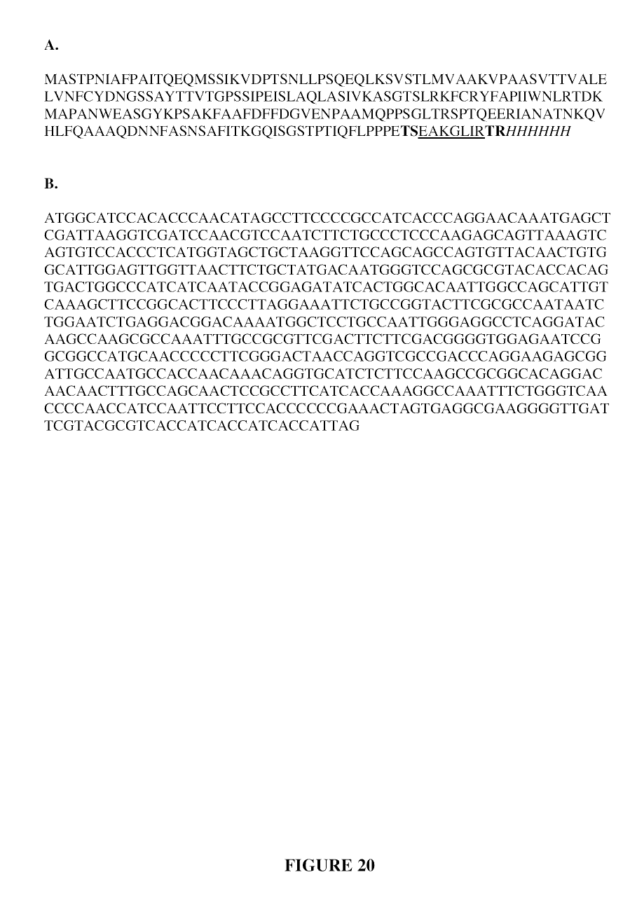

A further PapMV CP fusion comprising the OmpC affinity peptide EAKGLIR [SEQ ID NO:11] was constructed that included an additional 4 amino acids, 2 on each side of the affinity peptide (TR on the N-terminal side and TS on the C-terminal side, as shown in FIG. 19A). These amino acids are the result of the presence of the restriction sites SpeI-MluI that were used for the cloning of the affinity peptide in fusion with PapMV CP. The construct was designated “PapMV SM OmpC.” The complete amino acid sequence for the PapMV SM OmpC protein is provided in FIG. 20A (SEQ ID NO:8) and the nucleotide sequence encoding the PapMV SM OmpC protein is provided in FIG. 20B (SEQ ID NO:9).

The PapMV SM OmpC protein was purified as described in the preceding Examples (see FIG. 19B). The purified proteins showed VLP formation by electron microscopy, as expected (FIG. 19C).

### Immunizations

Balb/C mice, 10 mice per group, were immunised intraperitonally (I.P.) at day 0 as follows:

### Group 1: 10 μg OmpC

### Group 2: 10 μg OmpC+10 μg PapMV

### Group 3: 10 μg OmpC+10 μg PapMV OmpC

### Group 4: 10 μg PapMV

### Group 5: 10 μg PapMV OmpC

A second immunization (Boost) was performed at day 15 using 10 μg): OmpC in groups 1, 2 and 3. Group 4 was boosted with 10 μg): PapMV, and group 5 was boosted with 10 μg): PapMV OmpC. Challenge with S. typhi was performed on day 21. It was established experimentally that 90 000 CFU of S. typhi in mucin correspond to 1 LD₅₀. All mice were sacrificed at day 31.

### Results

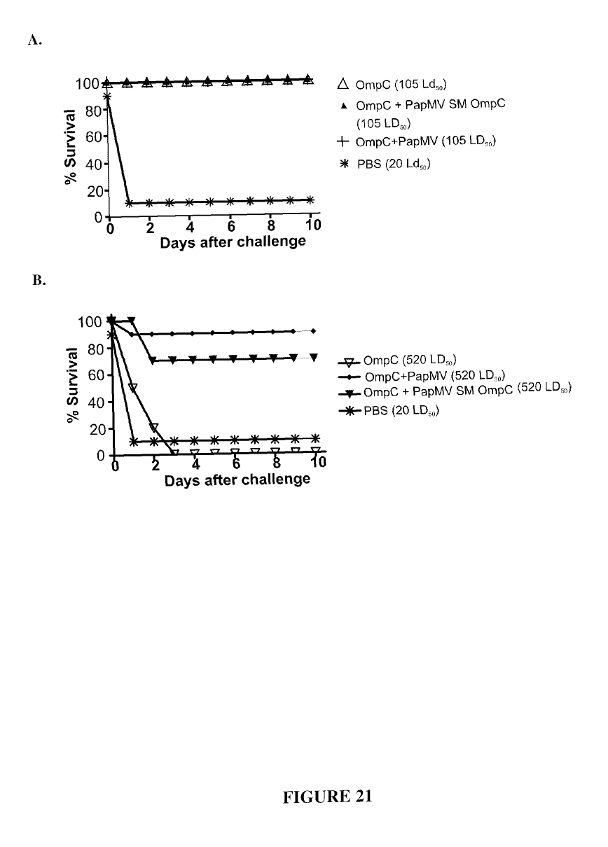

At 105LD₅₀ (FIG. 21A), all the preparations containing OmpC provided 100% protection. As expected, the mice vaccinated with preparations that did not contain OmpC all died within a few days of challenge with doses as low as 20LD₅₀. A difference was observed, however, between the OmpC-containing preparations when the mice were challenged with a higher dose of S. typhi (520LD₅₀). As can be seen from FIG. 21B, the most effective preparation at this dose was the combination of PapMV+OmpC in the first immunisation (Group 2) and a boost with OmpC alone. The preparation comprising PapMV SM OmpC VLPs conjugated to OmpC (Group 3) also performed well but appears to be slightly less effective than the PapMV+OmpC combination. However, whether or not the difference in efficacy was sufficient to be statistically significant could not be determined in this experiment.

In conclusion, as shown in the preceding Examples, the recombinant PapMV VLPs, with or without affinity peptides, are capable of acting as strong adjuvants that improve the protection capacity of two different antigens of S. typhi, OmpC and OmpF respectively.

### Example 10

### Adjuvant Effect o OmpC on a Commercial Influenza Vaccine Fluviral®

BALB/c mice were divided into 3 groups, 5 per group, and immunised once via the subcutaneous route as follows:
Group I: 3 μg (equivalent to one-fifth the human dose) of the commercial influenza vaccine Fluviral®.
Group II: 3 μg (equivalent to one-fifth the human dose) Fluviral® with 3 μg of purified OmpC.
Group III: 3 μg (equivalent to one-fifth the human dose) Fluviral® with 30 μg of purified OmpC.

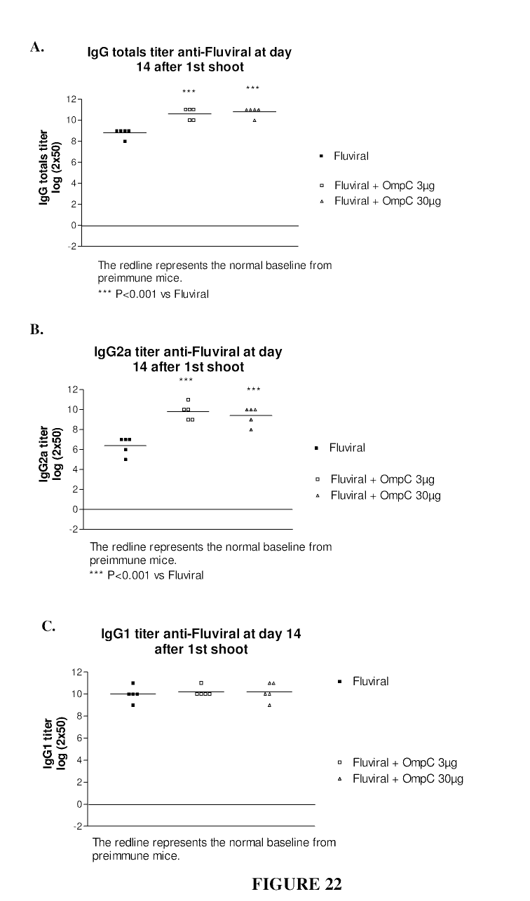

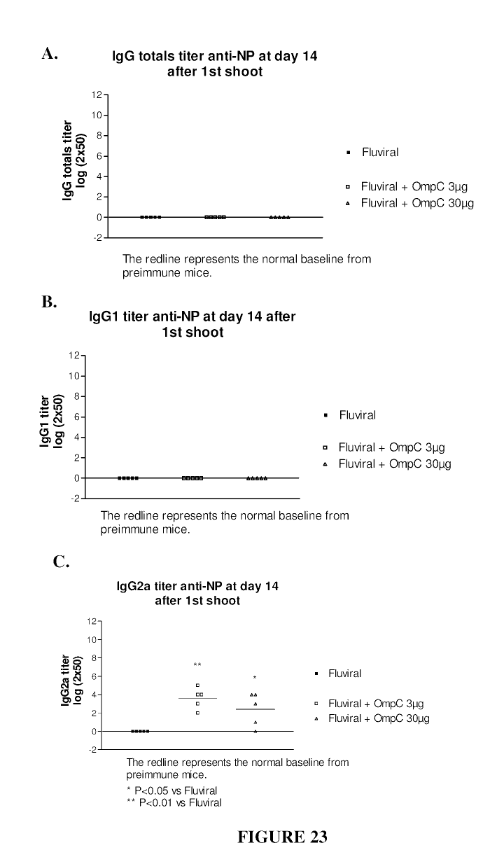

Blood was taken from the treated mice 14 days after injection and the total IgG was measured by ELISA toward the total Fluviral® proteins using peroxidase-conjugated goat anti-mouse IgG as secondary antibodies (Jackson ImmunoResearch Laboratories, Inc.). As can be seen from the results shown in FIG. 22A, the addition of as little as 3 μg of OmpC improves the immune response to the Fluviral® vaccine by 4-fold after only one injection.

The levels of IgG2a directed to the Fluviral® proteins were also measured by ELISA. The IgG class switch that induces the production of IgG2a is indicative of the stimulation of a TH1 response in mice. As such, the presence of IgG2a suggests the triggering of a CTL response. As shown in FIG. 22B, both adjuvant regimens (Group II and Group III mice) induced an amount of IgG2a that was increased by a factor of 8-fold over non-adjuvanted Fluviral®. This is a striking improvement of the adjuvanted regimens over Fluviral® alone.

The amount of IgG1 did not increase when OmpC was added to the Fluviral® vaccine (FIG. 22C). IgG1 is a marker for the TH2 response. As noted above, the improvement observed in the IgG2a profile suggests that OmpC triggers a TH1 response, which is consistent with the fact that the IgG1 levels are not influenced.

As noted in Example 3 (FIG. 6), the commercial Fluviral® vaccine is unable to induce an immune response to the conserved NP protein. Addition of either 3.1 g or 30 μg OmpC to the Fluviral® vaccine, while not improving the IgG1 titers (FIG. 23B), improved the IgG2a titers to this highly conserved target considerably (FIG. 23C). This result implies that a CTL response directed to NP was also triggered in those mice immunized with the Fluviral® vaccine adjuvanted with OmpC.

### Example 11

### Challenge of OmpC-Fluviral® Vaccinated Mice with Influenza Strain WSN/33

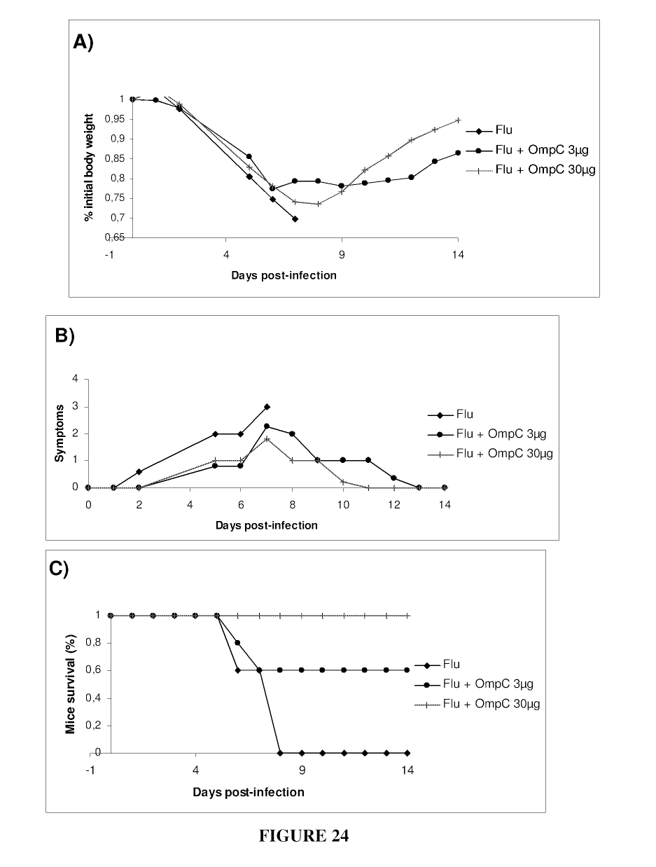

Mice vaccinated as described in Example 10 (Groups I-III) were challenged with 4,000 pfu (plaque forming units) of influenza strain WSN/33. All Group I mice (vaccinated with Fluviral® alone) were infected and showed a rapid decrease in body weight (FIG. 24A) and exhibited severe symptoms (FIG. 24B). The symptom legend for FIG. 24B is provided in Table 5 above. All Group I mice eventually died as a result of the infection (FIG. 24C).

In contrast, the mice vaccinated with Fluviral® adjuvanted with either 3 μg or 30 μg of OmpC lost less weight (FIG. 24A), showed less severe symptoms (FIG. 24B) and improved survival (FIG. 24C). While both groups of mice receiving the adjuvanted Fluviral® vaccine showed an improvement over those receiving the non-adjuvanted Fluviral® vaccine, the group receiving the higher dose of OmpC (30 μg) showed the best results with all mice surviving the infection. This result strongly suggests that the addition of OmpC to the Fluviral® vaccine triggered a CTL response in the mice toward highly conserved epitope of influenza, such as the NP protein. As a result, a complete protection to a lethal challenge of the WSN/33 strain of influenza was demonstrated.

### Example 12

### Use of PapMV VLPs Alone as an Adjuvant of the Fluviral® Vaccine #2

In this Example, PapMV VLPs comprising a modified coat protein CPfl3y were combined with the Fluviral® influenza vaccine and the ability of the vaccine to protect mice against an influenza challenge was measured. The coat protein CPfl3y contains a substitution of phenylalanine for tyrosine at position 13 of the coat protein as described in Laliberté Gagné, M E, et al. FEBS J., 2008 April; 275(7):1474-84 (Epub ahead of print: Feb. 25, 2008).

### Experimental Design:

3 groups of 5 Balb/C mice mice were inoculated subcutaneously with the following:
Group I: Fluviral® ⅕ of human dose
Group II: Fluviral® ⅕ of human dose+PapMV CPfl3y 3 μg
Group III: Fluviral® ⅕ of human dose+PapMV CPfl3y 30 μg

Animals were inoculated at day 0, and bleedings were done at day 0, day 14 and day 28. ELISAs were performed at day 14 to measure the total amount of IgG, the amount of IgG1 and the amount of IgG2a raised against Fluviral® or the purified influenza virus NP protein. At day 28, the mice were challenged with the heterologous influenza strain A/WSN/33 virus at 4LD₅₀ (=4000 pfu/mouse in a volume of 50 μl).

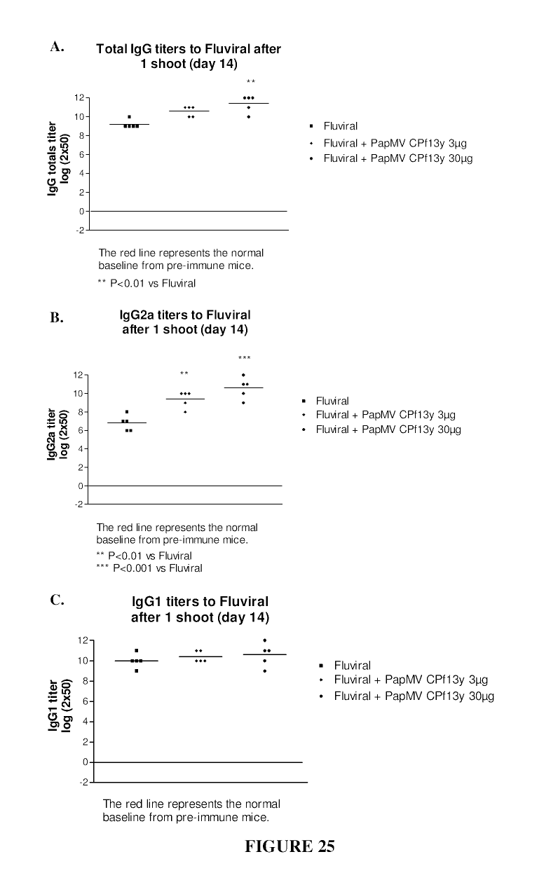

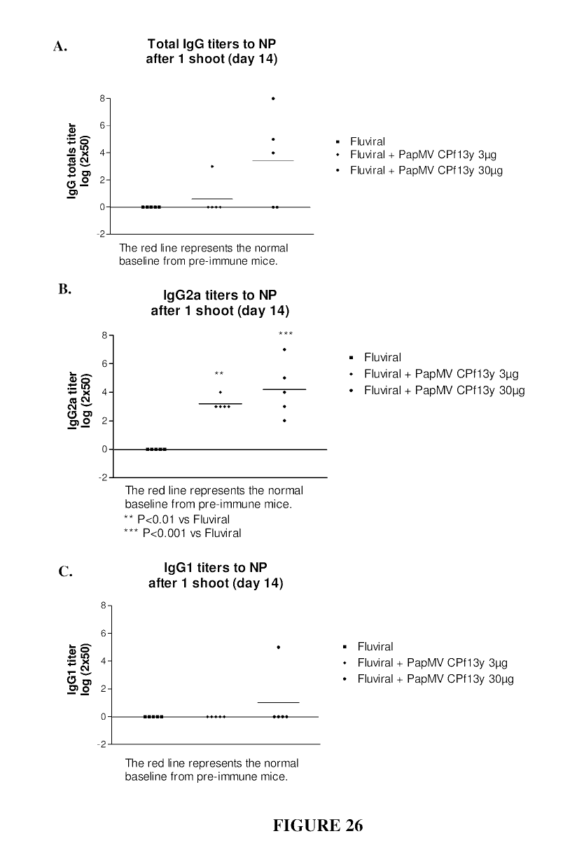

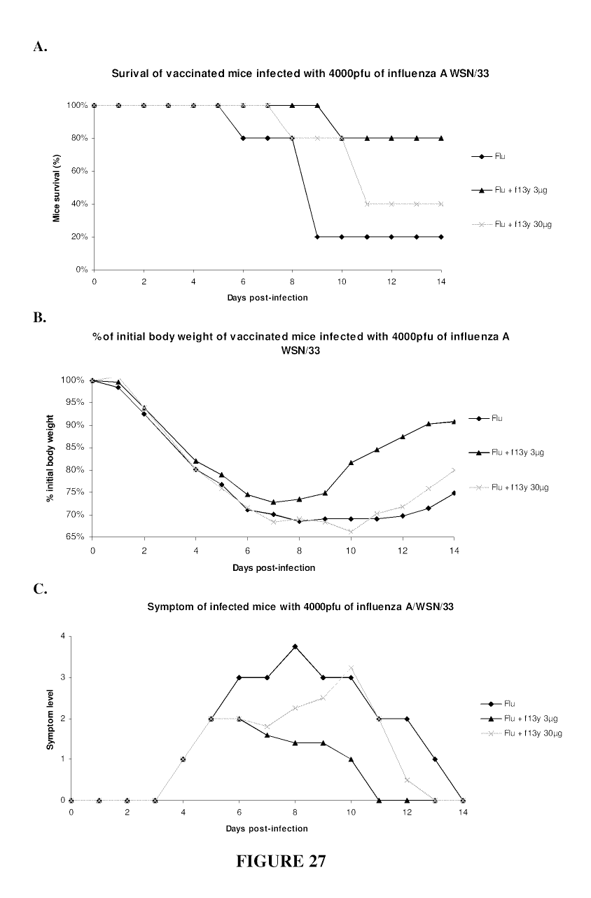

The results are presented in FIGS. 25 to 27, and show that the total IgG and the IgG2a response to the Fluviral® proteins (FIGS. 25A-B) and to the NP protein (FIGS. 26A-B) increased significantly with increasing amounts of PapMV VLPs The amount of IgG1 produced remained similar to that produced with Fluviral® alone (FIGS. 25C and 26C). This improvement in IgG2a correlated with a protection of 80% in the mice vaccinated with Fluviral®+3 μg of PapMV CPfl3y VLPs to the challenge with the WSN/33 strain of influenza (FIG. 27A). Interestingly, the mice vaccinated with Fluviral®+3 μg of PapMV CPfl3y VLPs showed better protection than those vaccinated with Fluviral®+30 μg of PapMV CPfl3y VLPs. The observed decrease in body weight and symptoms is shown in FIGS. 27B and C.

### Example 13

### Use of PapMV VLPs Alone as an Adjuvant of the Fluviral® Vaccine #3

This Example was conducted following a similar experimental protocol to that described in Example 12 above and using the 2008 version of the Fluviral® vaccine (which comprises the influenza strains: A/Solomon Islands/3/2006, A/Wisconsin/67/2005, B/Malaysia/2506/2004—see Example 3).

In brief, mice, 5 per group, were immunised once subcutaneously with ⅕ of the human dose of the trivalent flu vaccine Fluviral® (GSK) 2008 adjuvanted with 3 or 30 μg of the PapMV VLPs “rVLP-SM.” rVLP-SM comprise the coat protein PapMV CPsm (see Example 2). Bleeding were performed 14 days after the subcutaneous injection to analyse the immune response by ELISA. Mice were challenged with 4LD₅₀ of the heterologous influenza strain WSN/33.

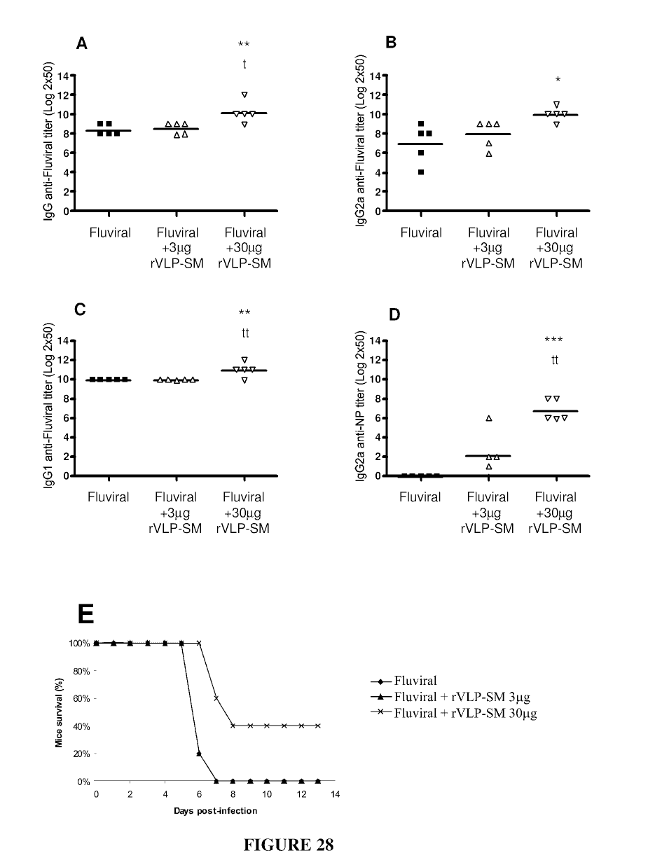

Fluviral® adjuvanted with rVLP-SM showed an improved humoral response to the vaccine and protection to a lethal influenza challenge. The results are shown in FIG. 28. (A) Total IgG to Fluviral® 2008 (log scale); (B) IgG2a to Fluviral® 2008 (log scale); (C) IgG1 to Fluviral® 2008 (log scale). (D) IgG2a measured directed at the purified influenza NP protein (log scale). The NP protein employed was a recombinant protein purified from E. coli using a 6×H tag by affinity on a Ni²⁺ column by a standard procedure. (E) Mice survival rate to challenge with 4LD₅₀ of the heterologous influenza strain WSN/33. Significant differences between the formulation adjuvanted with 30 μg of rVLP-SM and the Fluviral® 2008 treatment alone are shown by the symbol (*). Significant differences between the formulations containing 3 or 30 μg rVLP-SM are shown by the symbol (t). One symbol corresponds to a level of confidence of P<0.05, two symbols to P<0.01, and three symbols to P<0.0

The addition of the rVLP-SM to the Fluviral® vaccine can be seen to have significantly increased the humoral response to Fluviral proteins and NP. The induction of IgG2a toward NP suggests the induction of a TH1 cellular response toward this highly conserved influenza antigen. This improvement leads to a 40% protection toward a lethal dose of 4LD₅₀ of the WSN/33, an heterologous strain of influenza toward which the trivalent vaccine does not provide any protection.

### Example 14

### Use of PapMV VLPs Alone as an Adjuvant of the Fluviral® Vaccine in Primates

The PapMV VLPs “rVLP-SM” used in this Example comprise the coat protein PapMV CPsm (see Example 2).

Five macaques (Macaca fascicularis) were used in this Example and were divided into two groups:

Group 1: Two macaques immunized on day 0 and day 28 with intramuscular administrations of a human dose (15 μg) of Fluviral® 2008.

Group 2: Three macaques immunized on day 0 and day 28 with intramuscular administrations of a human dose (15 μg) of Fluviral® 2008 each time adjuvanted with 150 μg rVLP-SM.

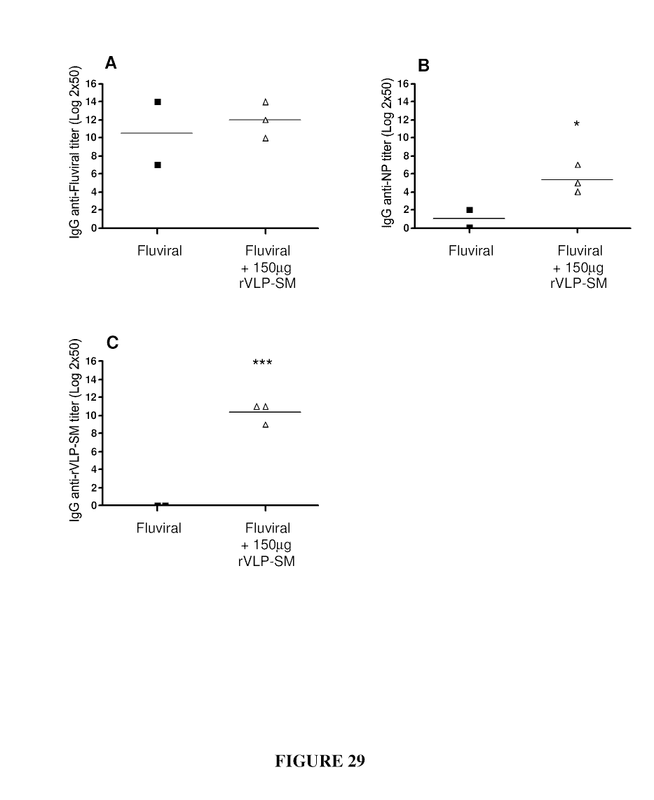

Animals were bled at day 56 and the immune response was analysed by ELISA. The results are shown in FIG. 29. (A) Total IgG response (log scale). (B) IgG response toward NP (log scale). (C) IgG response toward rVLP-SM (log scale). The NP was purified as described in Example 13.

The adjuvanted vaccine showed a significant improvement of the humoral response toward NP (*P<0.05) and rVLP-SM (***P<0.001). The results show that rVLP-SM is immunogenic in primates and can significantly improve the humoral response to the well conserved NP protein of Fluviral® 2008.

### Example 15

### Immune Response Triggered with Various PapMV VLP Dosage Regimens

The PapMV VLPs “rVLP-SM” used in this Example comprise the coat protein PapMV CPsm (see Example 2).

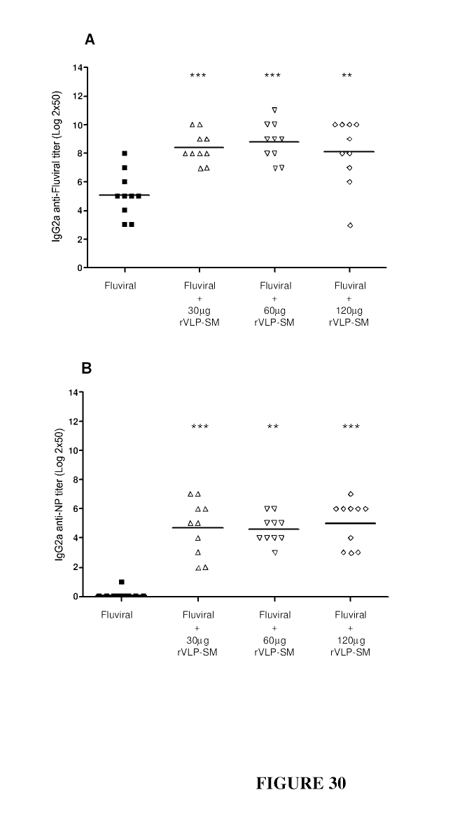

Balb/C mice (10 per group) were immunized with one subcutaneous injection of the Fluviral® 2008 vaccine alone (⅕ of a human dose, 3 μg), or Fluviral® 2008 adjuvanted with increasing amounts of rVLP-SM (30, 60 or 120 μg). Blood was collected 14 days after immunization and the humoral response was analysed by ELISA. The results are shown in FIG. 30: (A) IgG2a titer directed to Fluviral® 2008 (log scale), and (B) IgG2a titer directed to NP (log scale). Significant differences between the adjuvanted groups and the group treated with Fluviral® alone are shown by **P<0.01 and ***P<0.0001.

The results demonstrate that the immune response is saturated when as little as 30 μg of rVLP-SM are used to adjuvant the Fluviral® 2008 vaccine (i.e. a 1:10 by weight ratio of vaccine:VLPs).

### Example 16

### Use of PapMV VLPs Alone as an Adjuvant of Various Trivalent Influenza Vaccines

This Example was conducted following a similar experimental protocol to that described in Example 12 above. The PapMV VLPs “rVLP-SM” used in this Example comprise the coat protein PapMV CPsm (see Example 2). The influenza vaccines employed were the Fluviral® 2009 (GSK) or Influvac™ 2009 (Solvay) vaccines. Both these vaccines are trivalent and contain the influenza virus strains: A/Brisbane/59/2007 (H1N1); A/Brisbane/10/2007 (H₃N₂) and B/Florida/4/2006.

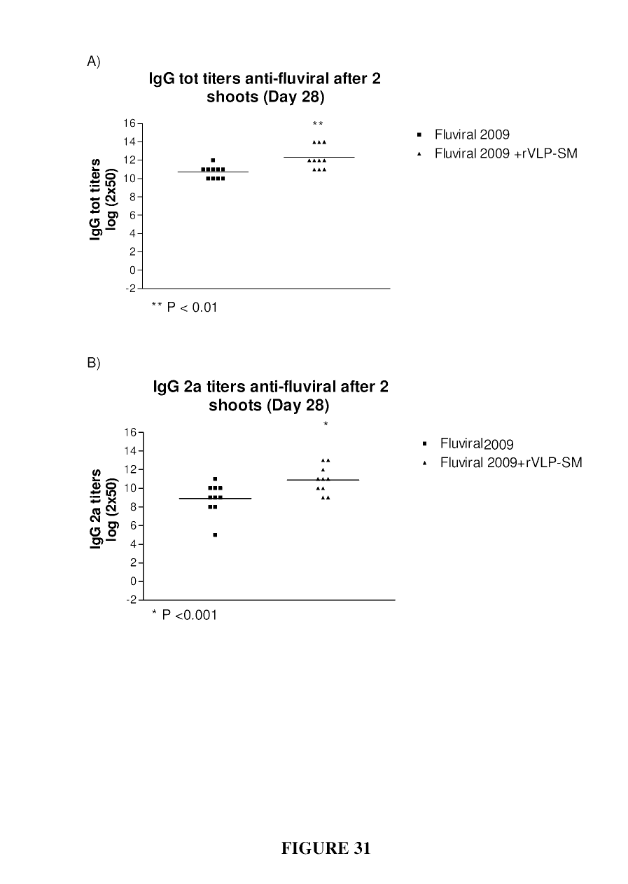

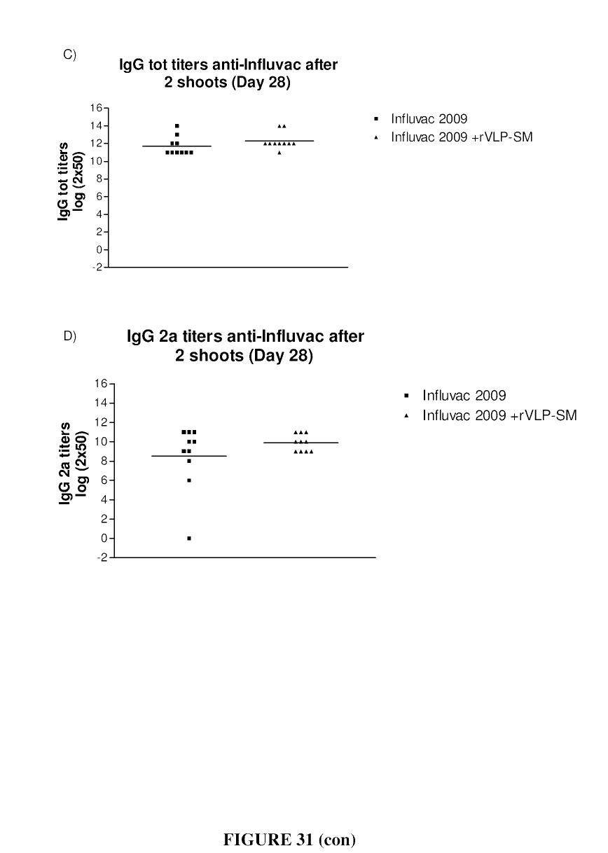

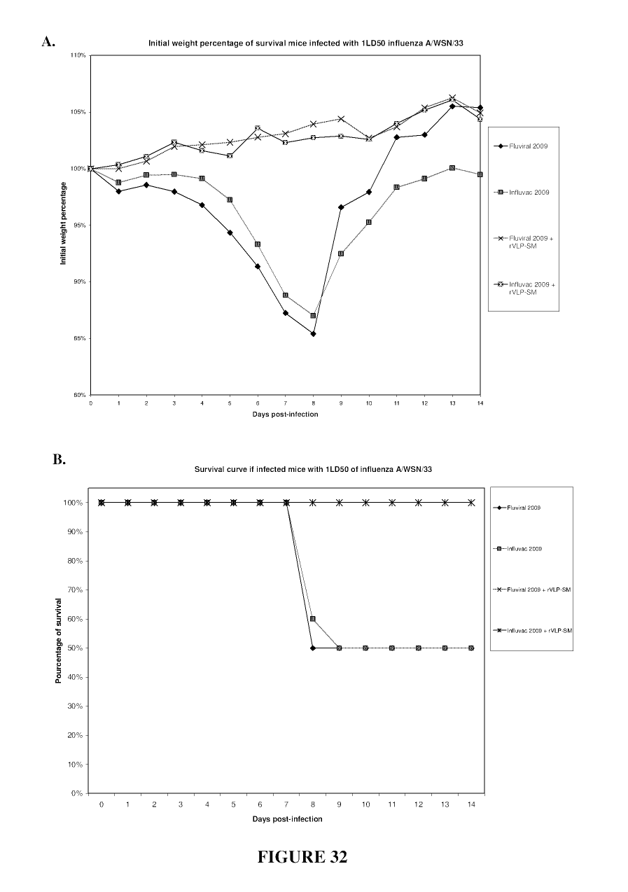

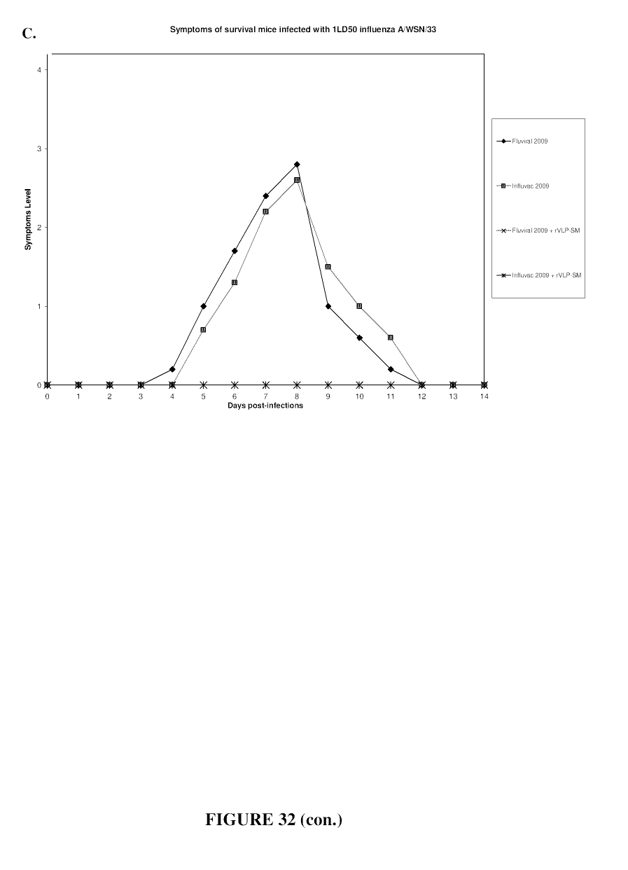

In brief, Balb/C mice (10 per group) were immunized twice at 14-day intervals with two subcutaneous injections of either the Fluviral® 2009 or the Influvac 2009 vaccine alone (⅕ of a human dose, 3 μg), or with one of the commercial vaccines adjuvanted with rVLP-SM 30 μg. Blood was collected at day 28 and the humoral response was analysed by ELISA. The results are shown in FIGS. 31 and 32.

FIG. 31: (A) total IgG titers directed to Fluviral® 2009 (log scale); (B) IgG2a titers directed to Fluviral® 2009; (C) total IgG titers directed to Influvac® 2009 (log scale) and (D) IgG2a titers directed to Influvac® 2009.

FIG. 32: (A) Weight curve, (B) survival curve, and (C) symptoms of mice vaccinated with Fluviral 2009, Influvac 2009 or the commercial vaccine adjuvanted with 30 μg of rVLP-SM then challenged with 1LD₅₀ of the heterologous influenza strain WSN/33.

Significant differences between the total IgG and IgG2a of the adjuvanted groups and the Fluviral® 2009 alone group were observed (see FIGS. 31A and B). A significant difference in the titers of total IgG and IgG2a between mice vaccinated with Influvac 2009 alone and the adjuvanted groups was not observed, however, cross-protection to challenge with a heterologous strain of influenza WSN/33 was observed for both groups of mice receiving adjuvanted vaccine, i.e. both adjuvanted Fluviral® and adjuvanted Influvac (FIG. 32B). The challenge was performed with 1LD₅₀ of the WSN/33 strain and a lack of weight loss, complete protection and absence of infection symptoms was observed only in those groups that received the adjuvanted vaccines. The addition of the adjuvant rVLP-SM to the commercial vaccines thus enables the vaccine to protect against heterologous strains.

### Example 17

### PapMV VLPs Improve the Efficacy of the Fluviral® Vaccine Through Induction of a Cellular Immune Response

This Example was conducted following a similar experimental protocol to that described in Example 16 above. The PapMV VLPs “rVLP-SM” used in this Example comprise the coat protein PapMV CPsm (see Example 2).

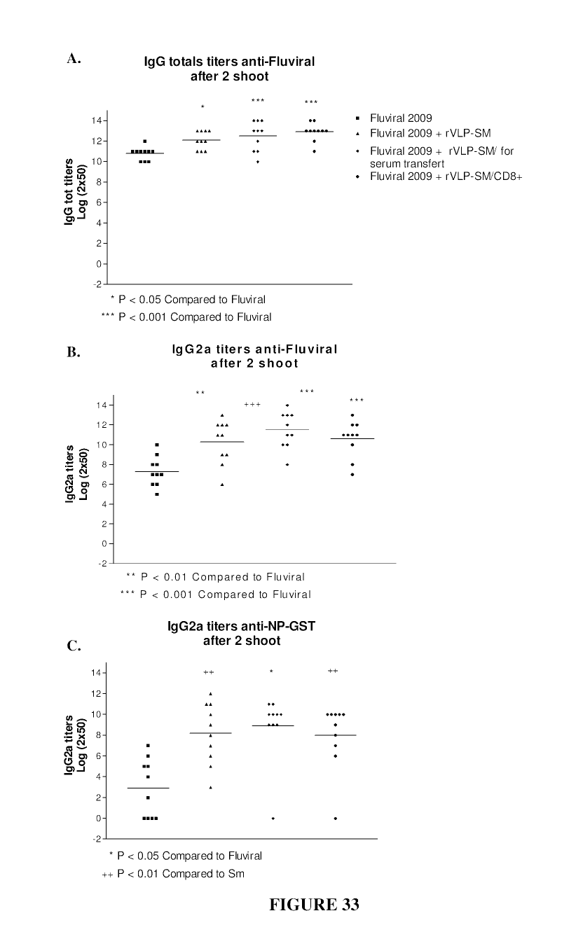

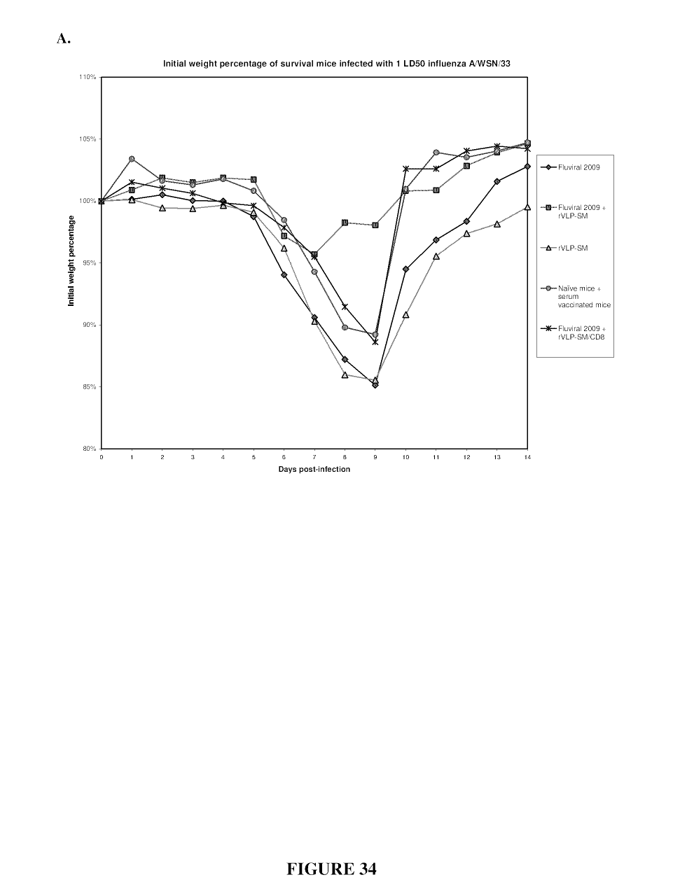

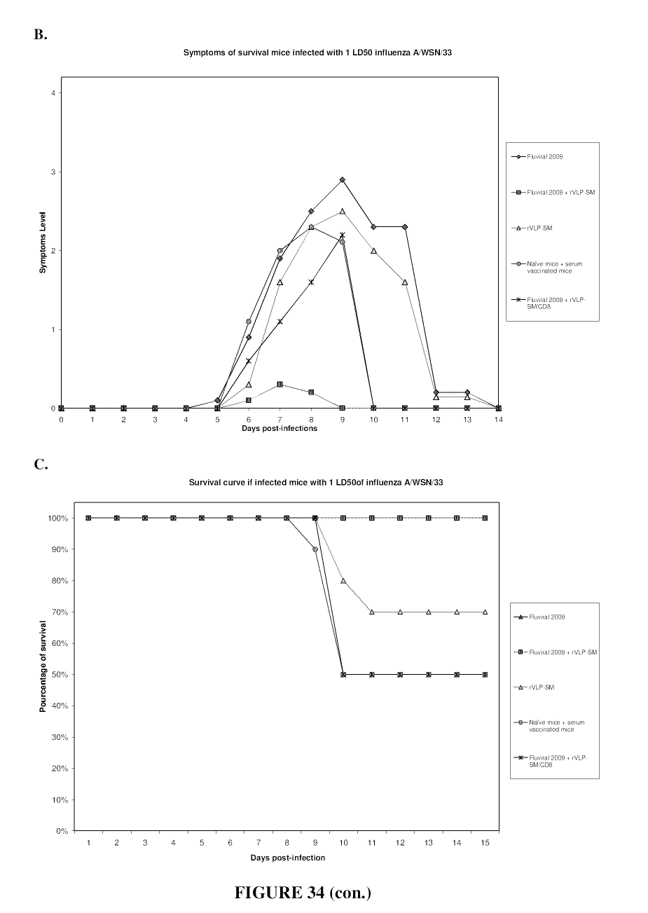

In brief, Balb/C mice (10 per group) were immunized twice at 14-day intervals with two subcutaneous injections of the Fluviral® 2009 (⅕ of a human dose, 3 μg) (one group), with the commercial vaccine adjuvanted with rVLP-SM 30 μg (3 groups) or with the adjuvant rVLP-SM 30 μg alone. The results are shown in FIGS. 33 and 34. The following treatments are represented:

Fluviral 2009: corresponds to mice vaccinated with the commercial vaccine alone and challenged on day 28 with 1LD₅₀ of influenza strain WSN/33.

Fluviral 2009+ rVLP-SM: corresponds to mice vaccinated with the commercial vaccine adjuvanted with 30 μg rVLP-SM and challenged on day 28 with 1LD₅₀ of influenza strain WSN/33.

rVLP-SM: corresponds to mice vaccinated with 30 μg rVLP-SM alone and challenged on day 28 with 1LD₅₀ of influenza strain WSN/33.

Naïve mice+serum vaccinated mice: corresponds to a group of 10 naïve mice that each received 250 μL of serum from mice previously vaccinated twice at 14-day intervals with Fluviral 2009+ rVLP-SM. The serum was administered by the intraperitoneal route at day 35 and the mice were challenged at day 36 with 1LD₅₀ of influenza strain WSN/33.

Fluviral 2009+ rVLP-SM/CD8+: corresponds to mice vaccinated with Fluviral 2009+ rVLP-SM twice at 14-day intervals. At days 33 and 35, 0.1 μg of monoclonal CD8+ rat antibodies were injected by the intraperitoneal route to deplete the CD8+ cells in the mice. At day 36, the mice were challenged with 1LD₅₀ of influenza strain WSN/33.

FIG. 33 shows the total IgG (A) and IgG2a (B) to Fluviral 2009 and the IgG2a to purified GST-NP (C) as measured by ELISA. GST-NP protein is a fusion protein of the glutathione S-transferase with the NP of the influenza stain WSN/33. The GST fusion facilitates the purification of the protein, which is used directly in the ELISA. The weight, symptoms and survival of the mice (FIGS. 34A, B and C, respectively) were measured every day during 14 days.

The results show clearly that the cellular response (CD8+ cells) is very important in protecting against a challenge with an heterologous influenza strain (WSN/33) because the depletion of the CD8+ T cells with CD8+ antibodies abolished the protective capacity of mice vaccinated with Fluviral 2009+ rVLP-SM 30 μg. This is further supported by the observation that serum isolated from vaccinated mice and re-administered to naïve mice was not sufficient to provide a high level of protection to the challenge. The results suggest that the cellular response must also be present and activated for full protection.

Taken together, the induction of IgG2a to NP and the lack of protection in vaccinated mice depleted in CD8+ strongly suggest that the adjuvant rVLP-SM is able to trigger a CTL response in mice to conserved influenza proteins like NP, which provide protection to challenge with an heterologous influenza strain. Thus, the rVLP-SM are able to improve the immune response to conserved influenza proteins like NP in animals vaccinated with commercial vaccines like Fluviral 2008, 2009 and Influvac 2009. This result in turn suggests that the addition of the adjuvant rVLP-SM will enable the adjuvanted vaccines to protect against most, if not all, strains of influenza.

### Example 18

### PapMV VLPs Trigger a Long-Lasting Immune Response

This Example was conducted following a similar experimental protocol to that described in Example 12 above. The PapMV VLPs “rVLP-SM” used in this Example comprise the coat protein PapMV CPsm (see Example 2).

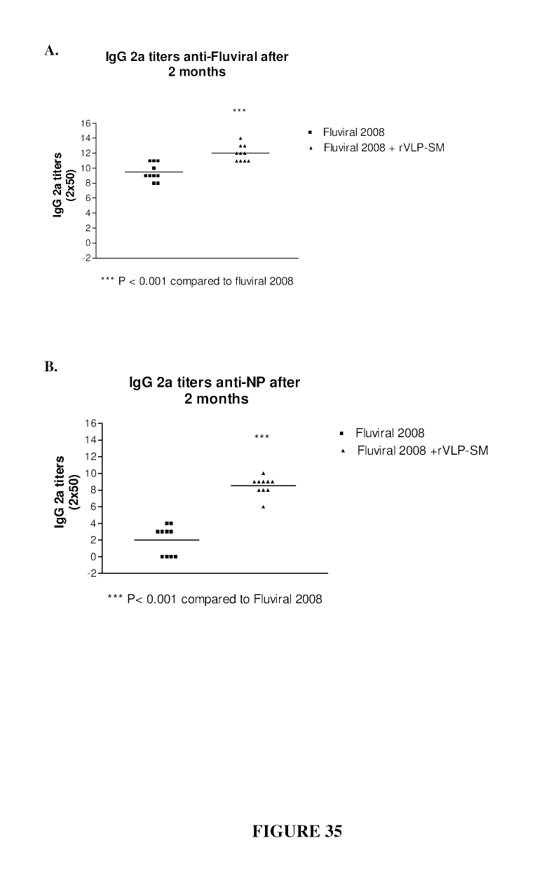

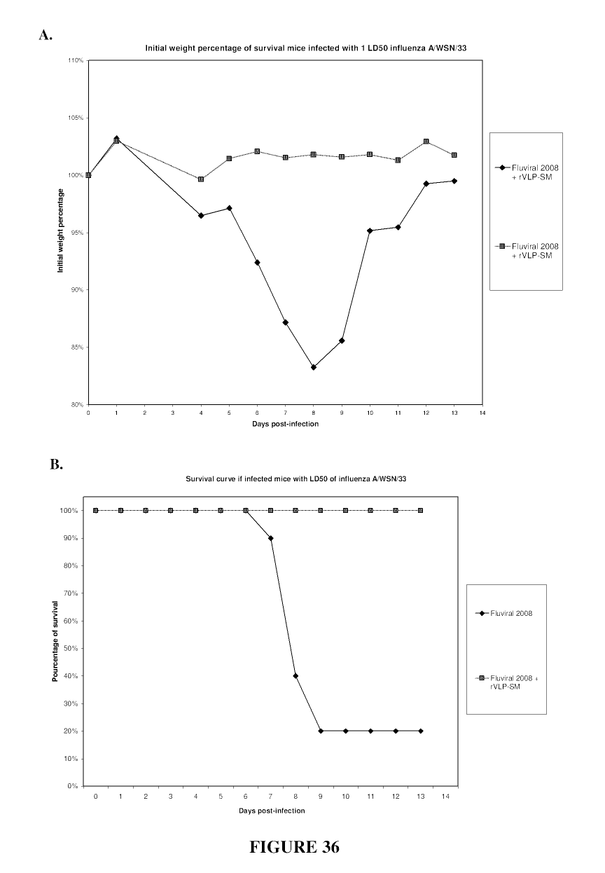

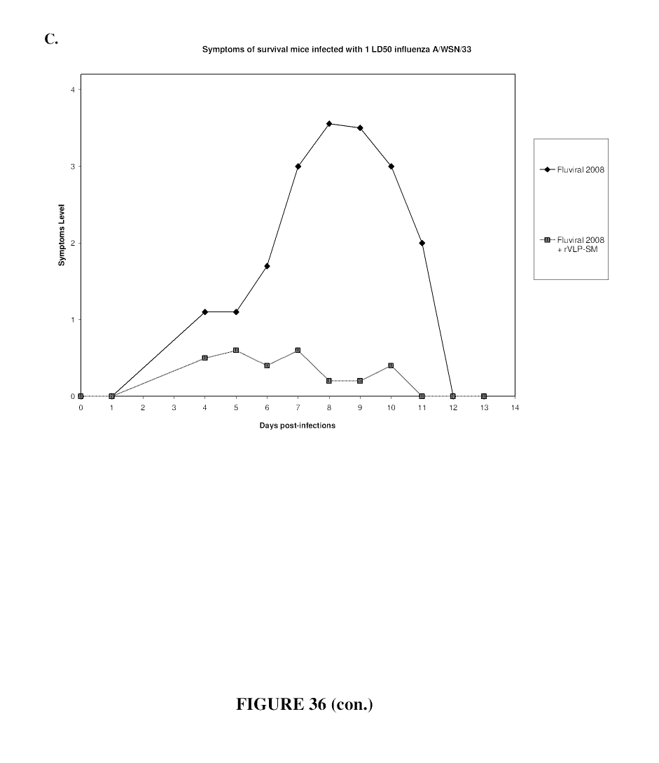

In brief, Balb/C mice (10 per group) were immunized once with one subcutaneous injection of the Fluviral® 2008 alone (⅕ of a human dose, 3 μg), or the commercial vaccine adjuvanted with rVLP-SM 30 μg. Blood was collected at day 56 and the humoral response was analysed by ELISA. Mice were challenged with 1LD₅₀ of WSN/33 10 months after the immunisation of the animals. The results are shown in FIGS. 35 and 36.

FIG. 35 shows (A) IgG2a titers directed to Fluviral® 2008 at 2 months after immunization, and (B) IgG2a titres directed to NP protein at 2 months after immunization. NP was purified as described in Example 13.

FIG. 36 shows (A) the weight curve, (B) the survival curve, and (C) the symptoms of mice vaccinated with Fluviral 2008, or with the commercial vaccine adjuvanted with 30 μg of rVLP-SM and then challenged with 1LD₅₀ of the heterologous influenza strain WSN/33.

As can be seen from FIG. 36, just one s.c. injection of the Fluviral 2008 vaccine adjuvanted with 30 μg of rVLP-SM was sufficient to confer protection on the animals against an heterologous strain of influenza ten months after the vaccination. This result shows the capacity of the adjuvant to trigger a long lasting memory response in mice that results in protection to challenge with an heterologous strain of influenza.

Overall the results shown in Examples 13 through 18 above demonstrate that the PapMV VLP adjuvant:
Improves the immune response of 3 different commercial vaccines, Fluviral® 2008, 2009 and Inflnvac™ 2009, which comprise different combinations of strains of influenza viruses, suggesting that the adjuvant is broadly applicable to the improvement of all influenza vaccines.
Improves the immune response to the conserved influenza protein NP and was able to provide 100% protection to a challenge with 1 LD₅₀ of an heterologous strain (WSN/33), suggesting that the addition of the adjuvant may be capable of making the adjuvanted vaccine universal and able to provide protection to all strains of influenza.
Improves the protection capacity of the vaccine via the cellular response.
Induces a long lasting memory response that provides protection to the vaccinated animal for more than 10 months after the administration of the vaccine.
Improves the immune response induced by a commercial influenza vaccine to the conserved influenza protein NP in large animals such as Macaca fascicularis (Macaque monkeys), which strongly suggests that the adjuvanted vaccine will also be efficient in humans.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A multivalent vaccine product comprising one or more antigens and a composition comprising a papaya mosaic virus (PapMV) component, and optionally a porin component,
said PapMV component comprising PapMV or PapMV virus-like particles (VLPs) derived from PapMV coat protein, and
said porin component comprising a Salmonella spp. OmpC, OmpF or a combination thereof.

2. The multivalent vaccine product according to claim 1, wherein said composition comprises PapMV VLPs and said one or more antigens are one or more influenza virus antigens.

3. The multivalent vaccine product according to claim 2, wherein said influenza virus antigens are provided in the form of a pre-formulated influenza vaccine.

4. The multivalent vaccine product according to claim 3, wherein said influenza virus antigens include influenza A antigens and influenza B antigens.

5. The multivalent vaccine product according to claim 3, wherein said pre-formulated influenza vaccine is an inactivated whole virion or split virion vaccine.

6. The multivalent vaccine product according to claim 3, wherein said pre-formulated influenza vaccine is a trivalent, split virion vaccine.

7. The multivalent vaccine product according to claim 3, wherein said pre-formulated influenza vaccine and said PapMV VLPs are present in a ratio of between about 1:1 and about 1:20 by weight.

8. The multivalent vaccine product according to claim 3, wherein said pre-formulated influenza vaccine and said PapMV VLPs are present in a ratio of between about 1:5 and about 1:15 by weight.

9. The multivalent vaccine product according to claim 2, wherein said composition further comprises a porin component.

10. The multivalent vaccine product according to claim 1, wherein said composition comprises a porin component.

11. The multivalent vaccine product according to claim 10, wherein said porin component is conjugated to the coat protein of said PapMV or VLP.

12. The multivalent vaccine product according to claim 11, wherein said porin component is attached by affinity binding to said coat protein.

13. The multivalent vaccine product according to claim 10, wherein said porin component is not conjugated to said PapMV or VLP.

14. The multivalent vaccine product according to claim 10, wherein said porin component comprises OmpC.

15. The multivalent vaccine product according to claim 10, wherein said PapMV component comprises PapMV VLPs.

16. The multivalent vaccine product according to claim 10, wherein said PapMV component and said porin component are present in a ratio of between about 20:1 and about 1:10 by weight.

17. The multivalent vaccine product according to claim 10, wherein said one or more antigens are provided in the form of a pre-formulated vaccine.

18. The multivalent vaccine product according to claim 17, wherein said one or more antigens are influenza virus antigens.

19-54. (canceled)

55. A method of enhancing an immune response against one or more pathogens in an animal, said method comprising administering to said animal one or more antigens and an effective amount of a composition comprising a papaya mosaic virus (PapMV) component, and optionally a porin component,
said PapMV component comprising PapMV or PapMV virus-like particles (VLPs) derived from PapMV coat protein, and
said porin component comprising a Salmonella spp. OmpC, OmpF or a combination thereof.

56. The method according to claim 55, wherein said composition comprises PapMV VLPs and said one or more antigens are one or more influenza virus antigens.

57. The method according to claim 56, wherein said influenza virus antigens are provided in the form of a pre-formulated influenza vaccine.

58. The method according to claim 56, wherein said influenza virus antigens include influenza A antigens and influenza B antigens.

59. The method according to claim 57, wherein said pre-formulated influenza vaccine is an inactivated whole virion or split virion vaccine.

60. The method according to claim 57, wherein said pre-formulated influenza vaccine is a trivalent, split virion vaccine.

61. The method according to claim 57, wherein said pre-formulated influenza vaccine and said PapMV VLPs are administered to said animal in a ratio of between about 1:1 and about 1:20 by weight.

62. The method according to claim 57, wherein said pre-formulated influenza vaccine and said PapMV VLPs are administered to said animal in a ratio of between about 1:5 and about 1:15 by weight.

63. The method according to claim 56, wherein said composition further comprises a porin component.

64. The method according to claim 55, wherein said composition comprises a porin component.

65. The method according to claim 64, wherein said porin component is conjugated to the coat protein of said PapMV or VLP.

66. The method according to claim 65, wherein said porin component is attached by affinity binding to said coat protein.

67. The method according to claim 64, wherein said porin component is not conjugated to said PapMV or VLP.

68. The method according to claim 64, wherein said porin component comprises OmpC.

69. The method according to claim 64, wherein said PapMV component comprises PapMV VLPs.

70. The method according to claim 64, wherein said PapMV component and said porin component are present in a ratio of between about 20:1 and about 1:10 by weight.

71. The method according to claim 64, wherein said one or more antigens are provided in the form of a pre-formulated vaccine.

72. The method according to claim 71, wherein said one or more antigens are influenza virus antigens.

73. The method according to claim 64, wherein said immune response is against a plurality of pathogens.

74. The method according to claim 73, wherein one of said plurality of pathogens is S. typhi.

75. The method according to claim 73, wherein one of said plurality of pathogens is an influenza virus.

76. The method according to claim 56, wherein said immune response is against influenza virus.

77. The method according to claim 57, wherein said immune response is against influenza virus and comprises an immune response to a conserved influenza antigen.

78. The method according to claim 77, wherein said immune response comprises a CTL immune response to a conserved influenza antigen.

79. The method according to claim 76, wherein said immune response provides protection against a plurality of influenza strains.

80. The method according to claim 55, wherein said immune response comprises humoral and cellular immune responses.

81. The method according to claim 55, wherein said animal is a human.

82-87. (canceled)

88. A method of improving the efficacy of an influenza vaccine comprising administering to a subject said influenza vaccine and a composition comprising PapMV virus-like particles (VLPs) and optionally a porin component, whereby the subject treated with said influenza vaccine and said composition shows an improved immune response over a subject treated with said influenza vaccine alone, wherein said porin component comprises a Salmonella spp. OmpC, OmpF or a combination thereof.

89. The method according to claim 88, wherein said improved immune response comprises a cellular immune response.

90. The method according to claim 88, wherein said improved immune response comprises an immune response to a conserved influenza antigen.

91. The method according to claim 88, wherein said improved immune response comprises a CTL immune response to a conserved influenza antigen.

92. The method according to claim 88, wherein said improved immune response confers protection against one or more heterologous strains of influenza.

93. The method according to claim 88, wherein said subject is a human.

94. The method according to claim 88, wherein said influenza vaccine comprises influenza A antigens and influenza B antigens.

95. The method according to claim 88, wherein said influenza vaccine is an inactivated whole virion or split virion vaccine.

96. The method according to claim 88, wherein said influenza vaccine is a trivalent, split virion vaccine.

97. The method according to claim 88, wherein said influenza vaccine and said PapMV VLPs are administered to said subject in a ratio of between about 1:1 and about 1:20 by weight.

98. The method according to claim 88, wherein said influenza vaccine and said PapMV VLPs are administered to said subject in a ratio of between about 1:5 and about 1:15 by weight.

99. The method according to claim 88, wherein said influenza vaccine and said PapMV VLPs are administered to said subject as separate formulations.

100. The method according to claim 88, wherein said influenza vaccine and said PapMV VLPs are administered to said subject as a single formulation.

101. The multivalent vaccine product according to claim 1, wherein said one or more antigens and said composition are provided as separate formulations.

102. The multivalent vaccine product according to claim 1, wherein said one or more antigens and said composition are provided as a single formulation.

103. The method according to claim 55, wherein said one or more antigens and said composition are administered to said animal as separate formulations.

104. The method according to claim 55, wherein said one or more antigens and said composition are administered to said animal as a single formulation.

105. The method according to claim 77, wherein said immune response provides protection against one or more heterologous strains of influenza.

106. The method according to claim 105, wherein said animal is a human.


SEQUENCE LISTING (38 sequences)

SEQ ID NO: 1 - protein, 215 aa, Papaya Mosaic Virus
MSKSSMSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLIRSPTQEERIANATNKQVHLFQAAAQDNNFTSNSAFITKGQISGSTPTIQFLPPPE

SEQ ID NO: 2 - DNA, 648 bp, Papaya Mosaic Virus
atgtctaagtcaagtatgtccacacccaacatagccttccccgccatcacccaggaacagatgagctcgattaaggtcgatccaacgtccaatcttctgccctcccaagagcagttaaagtcagtgtccaccctcatggtagctgctaaggttccagcagccagtgttacaactgtggcattggagttggtcaacttctgctatgacaatgggtccagcgcgtacaccacagtgactggcccatcatcaataccggagatatcactggcacaattggctagtattgtcaaagcttccggcacttcccttagaaaattctgccggtacttcgcgccaataatctggaatctgaggacggacaaaatggctcctgccaattgggaggcttcaggatacaagccaagcgccaaatttgccgcgttcgacttcttcgacggggtggagaatccggcggccatgcaacccccttcgggactaatcaggtcgccgacccaggaagagcggattgccaatgctaccaacaaacaggtgcatctcttccaagccgcggcacaggacaacaactttaccagcaactccgccttcatcaccaaaggccaaatttctgggtcaaccccaaccatccaattccttccaccccccgaataa

SEQ ID NO: 3 - protein, 211 aa, Artificial Sequence
  PapMV modified coat protein CPdeltaN5
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPE

SEQ ID NO: 4 - protein, 378 aa, Salmonella enterica subsp. enterica serovar Typhi Ty2
MKVKVLSLLVPALLVAGAANAAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 5 - protein, 363 aa, Salmonella enterica subsp. enterica serovar Typhi CT18
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 6 - protein, 224 aa, Artificial Sequence
  PapMV coat protein + OmpC affinity peptide
MASTPNIAFPAITQEQMSSIEVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQGNNFASNSAFITKGQISGSTPTIQFLPPPEEAKGLIRHHHHHH

SEQ ID NO: 7 - protein, 224 aa, Artificial Sequence
  PapMV coat protein + OmpF affinity peptide
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPEFHENWPSHHHHHH

SEQ ID NO: 8 - protein, 228 aa, Artificial Sequence
  PapMV SM coat protein + OmpC affinity peptide
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSEAKGLIRTRHHHHHH

SEQ ID NO: 9 - DNA, 687 bp, Artificial Sequence
  sequence encoding PapMV SM coat protein + OmpC affinity peptide
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtgaggcgaaggggttgattcgtacgcgtcaccatcaccatcaccattag

SEQ ID NO: 10 - protein, 7 aa, Artificial Sequence
  Affinity peptide
SLSLIQT

SEQ ID NO: 11 - protein, 7 aa, Artificial Sequence
  Affinity peptide
EAKGLIR

SEQ ID NO: 12 - protein, 7 aa, Artificial Sequence
  Affinity peptide
TATYLLD

SEQ ID NO: 13 - protein, 7 aa, Artificial Sequence
  Affinity peptide
FHENWPS

SEQ ID NO: 14 - protein, 7 aa, Artificial Sequence
  Affinity peptide
FHEFWPT

SEQ ID NO: 15 - protein, 7 aa, Artificial Sequence
  Affinity peptide
FHEXWPT

SEQ ID NO: 16 - protein, 23 aa, Artificial Sequence
  M2e peptide from Human H1N1 e.g. A/USRR/90/77 and A/WSN/33
SLLTEVETPIRNEWGCRCNDSSD

SEQ ID NO: 17 - protein, 23 aa, Artificial Sequence
  M2e peptide
SLLTEVETPIRNEWGCRCNGSSD

SEQ ID NO: 18 - protein, 23 aa, Artificial Sequence
  M2e peptide
SLLTEVETPTKNEWDCRCNDSSD

SEQ ID NO: 19 - protein, 23 aa, Artificial Sequence
  M2e peptide from Equine H3N8 A/equine/Massachussetts/ 213/2003
SLLTEVETPTRNGWECKCSDSSD

SEQ ID NO: 20 - protein, 23 aa, Artificial Sequence
  M2e peptide from H5N1 A/Vietnam/1196/04
SLLTEVETPTRNEWECRCSDSSD

SEQ ID NO: 21 - protein, 24 aa, Artificial Sequence
  M2e peptide from Human H1N1 e.g. A/USRR/90/77 and A/WSN/33
MSLLTEVETPIRNEWGCRCNDSSD

SEQ ID NO: 22 - protein, 24 aa, Artificial Sequence
  M2e peptide from Avian H5N1 A/Vietnam/1196/04
HSLLTEVETPTRNEWECRCSDSSD

SEQ ID NO: 23 - protein, 24 aa, Artificial Sequence
  M2e peptide from H3N8, Horse-Dog A/equine/Massachussetts/ 213/2003
MSLLTEVETPTRNGWECKCSDSSD

SEQ ID NO: 24 - protein, 24 aa, Artificial Sequence
  M2e peptide from H9N2, A/chicken/Osaka/aq69/2001
MSLLTEVETPTRNGWGCRCSDSSD

SEQ ID NO: 25 - protein, 24 aa, Artificial Sequence
  M2e peptide from Mutant H1N1 I/T
MSLLTEVETPTRNEWGCRCSDSSD

SEQ ID NO: 26 - protein, 8 aa, Artificial Sequence
  M2e peptide variant
EVETPIRN

SEQ ID NO: 27 - protein, 8 aa, Artificial Sequence
  M2e peptide variant
EVETLTRN

SEQ ID NO: 28 - protein, 8 aa, Artificial Sequence
  M2e peptide variant
EVETPIRS

SEQ ID NO: 29 - protein, 8 aa, Artificial Sequence
  M2e peptide variant
EVETPTRN

SEQ ID NO: 30 - protein, 8 aa, Artificial Sequence
  M2e peptide variant
EVETPTKN

SEQ ID NO: 31 - protein, 8 aa, Artificial Sequence
  M2e peptide variant
EVDTLTRN

SEQ ID NO: 32 - protein, 8 aa, Artificial Sequence
  M2e peptide variant
EVETPIRK

SEQ ID NO: 33 - protein, 8 aa, Artificial Sequence
  M2e peptide variant
EVETLTKN

SEQ ID NO: 34 - DNA, 45 bp, Artificial Sequence
  Forward primer
atcgccatggcatccacacccaacatagccttccccgccatcacc

SEQ ID NO: 35 - DNA, 76 bp, Artificial Sequence
  Reverse Primer
ggttaaggaaggtggggggcttctccgcttccccaactaagcatggtagtggtagtggtaatcattcctaggtgac

SEQ ID NO: 36 - DNA, 77 bp, Artificial Sequence
  Reverse Primer
ggttaaggaaggtggggggcttaaagtactcttaaccggaagcgtggtagtggtagtggtaatcattcctaggtgac

SEQ ID NO: 37 - protein, 222 aa, Artificial Sequence
  PapMV modified coat protein PapMV CPsm
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTTRHHHHHH

SEQ ID NO: 38 - DNA, 669 bp, Artificial Sequence
  Sequence encoding PapMV modified coat protein PapMV CPsm
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtaccacgcgtcaccatcaccatcaccattag